(19) United States
(12) Patent Application Publication
(10) Pub. No.: US 2025/0057999 A1
(43) Pub. Date: Feb. 20, 2025

(54) GLUTAMIC ACID -UREA COMPOUND, PREPARATION METHOD AND USE THEREOF, NUCLIDE-TARGETED PROBE, PREPARATION METHOD AND USE THEREOF, AND PHARMACEUTICAL COMPOSITION

(71) Applicant: XIAMEN UNIVERSITY, Xiamen (CN)

(72) Inventors: Zhide GUO, Xiamen (CN); Xingxing CHENG, Xiamen (CN); Xianzhong ZHANG, Xiamen (CN); Hongwu LIU, Xiamen (CN); Xueqi WANG, Xiamen (CN)

(21) Appl. No.: 18/893,474

(22) Filed: Sep. 23, 2024

Related U.S. Application Data
(63) Continuation of application No. PCT/CN2023/128478, filed on Oct. 31, 2023.

(30) Foreign Application Priority Data
Aug. 17, 2023 (CN) 202311035883.5

(51) Int. Cl. A61K 51/08; A61P 35/00

(52) U.S. Cl. CPC A61K 51/088; A61P 35/00; A61K 2121/00; A61K 2123/00

(57) ABSTRACT

Provided is a glutamic acid (Glu)-urea compound and a preparation method and use thereof. The Glu-urea compound and the nuclide-targeted probe have excellent in vivo biological properties and show a high specific uptake in lesions with high expression of a prostate-specific membrane antigen (PSMA) protein. The compound and probe have a high target/non-target ratio, low non-specific background activity, and significantly-enhanced tumor uptake and retention time. The compound and probe are suitable for use in the nuclide therapy and imaging of tumors, and can also reduce unnecessary radiation damage to normal tissues and organs. The compound and probe can also overcome the shortcomings such as low target organ uptake and short retention time of small-molecule PSMA, and improve the effects of PSMA-targeted nuclide therapy and imaging, thus exhibiting a potential to be widely used in clinical applications.

### CROSS-REFERENCE TO RELATED APPLICATION

This patent application is a bypass continuation application of International Patent Application No: PCT/CN2023/128478, filed on Oct. 31, 2023, which claims the benefit and priority of Chinese Patent Application No. CN2023110358835 filed with the China National Intellectual Property Administration on Aug. 17, 2023, the disclosures of both of which are incorporated by reference herein in its entirety as part of the present application.

### TECHNICAL FIELD

The present disclosure belongs to the technical field of biomedicine, and specifically relates to a glutamic acid (Glu)-urea compound, a preparation method and use thereof, a pharmaceutically acceptable salt of the Glu-urea compound, a nuclide-targeted probe and a preparation method and use thereof, a pharmaceutically acceptable salt of the nuclide-targeted probe, and a pharmaceutical composition.

### BACKGROUND

Prostate-specific membrane antigen (PSMA) is a transmembrane glycoprotein that is overexpressed in approximately 90% of prostate cancers. Therefore, PSMA-targeted radioligand therapy (RLT) has emerged as a potentially valuable therapeutic strategy for metastatic castration-resistant prostate cancer (mCRPC). In addition, the expression of PSMA has also been found in other solid tumors, therefore, PSMA can be used as a therapeutic target for various tumors. Various PSMA-targeted radioligands have been developed in recent years, which have shown desirable promise in early clinical evaluations. The leader in this field is [¹⁷⁷Lu]Lu-PSMA617, and multiple current studies have shown that the [¹⁷⁷Lu]Lu-PSMA617 RLT exhibits excellent safety and efficacy in a large number of mCRPC patients. Nonetheless, single doses tend to be higher since the drug is metabolized quickly in vivo. Moreover, many patients respond inadequately to RLT and may experience disease progression during or after the treatment.

One strategy to enhance a therapeutic efficacy is to improve the delivery of radioligands. Currently, a commonly-used method is to combine an albumin-binding group with a PSMA-targeted radioligand to prolong the blood residence time, thereby increasing an uptake of radioligand by the tumor. For example, albumin-binding groups such as p-iodophenylbutyric acid, Evans blue, or ibuprofen can be modified into PSMA probes to achieve higher tumor uptake. However, this strategy generally also increases the irradiation dose to healthy organs and tissues, including the kidney and bone marrow. Therefore, the pharmacokinetic properties of the probe in vivo must be carefully controlled. In summary, it is of great significance to develop a PSMA-targeted probe with short blood circulation cycle, high tumor uptake, long-lasting lesion retention, and low background in non-target organs.

Deferasirox (DFX) is an oral iron chelator commonly used to reduce iron levels in patients with transfusion-dependent anemia and non-transfusion-dependent thalassemia. Studies have shown that DFX has certain tumor targeting properties, showing an effect of anti-tumor cell proliferation after being enriched in tumor sites. It has also been reported that DFX can be used as a chemotherapeutic drug, an antifungal drug, and an antibacterial drug. However, it is a difficulty facing scientific researchers to modify DFX to achieve better tumor targeting, higher lesion enrichment rate, and better disease treatment effect. In addition, the value of DFX structures has not yet been explored in the design of nuclide-targeted drugs. The impact of DFX hybridization with other receptor-targeting groups on pharmacokinetic properties of the probe is still unknown, and DFX remains to be explored in the field of nuclide-targeted diagnosis and treatment.

### SUMMARY

In view of this, an objective of the present disclosure is to provide a glutamic acid (Glu)-urea compound and a preparation method and use thereof, a pharmaceutically acceptable salt of the Glu-urea compound, a nuclide-targeted probe and a preparation method and use thereof, a pharmaceutically acceptable salt of the nuclide-targeted probe, and a pharmaceutical composition. The Glu-urea compound and a pharmaceutically acceptable salt thereof, as well as the nuclide-targeted probe and a pharmaceutically acceptable salt thereof in the present disclosure each have a PSMA-targeting performance, a long retention time in target organ, a high tumor uptake dose, and a low background.

To achieve the above objective, the present disclosure provides the following technical solutions:

The present disclosure provides a glutamic acid (Glu)-urea compound, where the Glu-urea compound has a structure shown in formula I-1 or formula I-2:

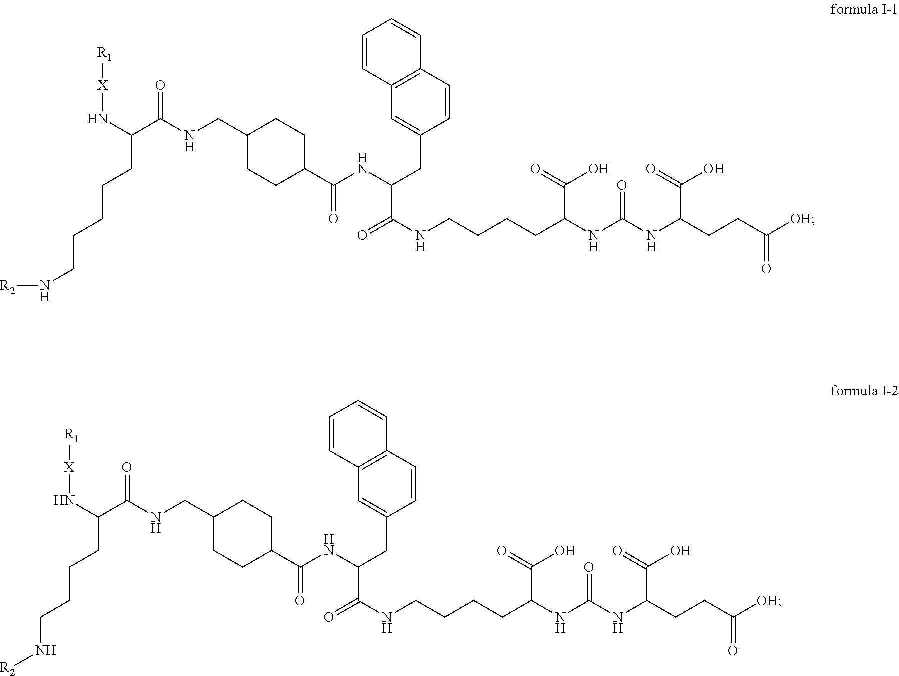

wherein one of R₁ and R₂ is selected from the group consisting of

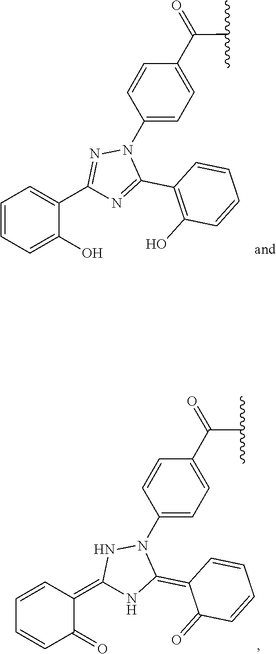 [SMILES: *C(=O)c1ccc(-n2nc(-c3ccccc3O)nc2-c2ccccc2O)cc1 ; *C(=O)c1ccc(N2N/C(=C3\C=CC=CC3=O)N/C2=C2/C=CC=CC2=O)cc1]

while the other is a group to be labeled, and the group to be labeled is one selected from the group consisting of:

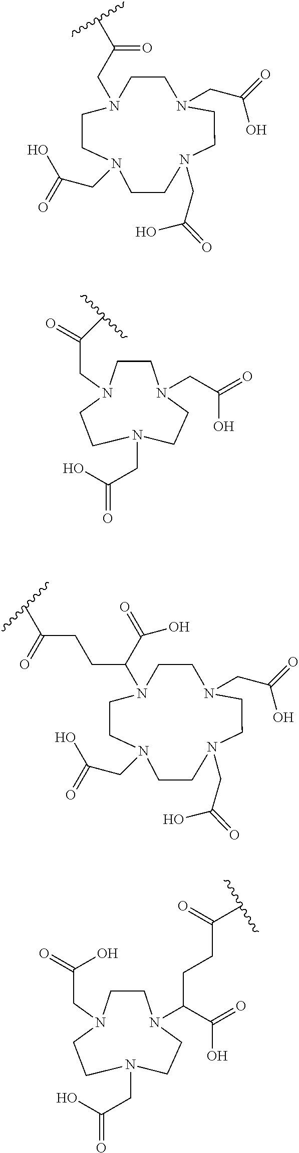 [SMILES: *C(=O)CN1CCN(CC(=O)O)CCN(CC(=O)O)CCN(CC(=O)O)CC1 ; *C(=O)CN1CCN(CC(=O)O)CCN(CC(=O)O)CC1 ; *C(=O)CCC(C(=O)O)N1CCN(CC(=O)O)CCN(CC(=O)O)CCN(CC(=O)O)CC1 ; *C(=O)CCC(C(=O)O)N1CCN(CC(=O)O)CCN(CC(=O)O)CC1]

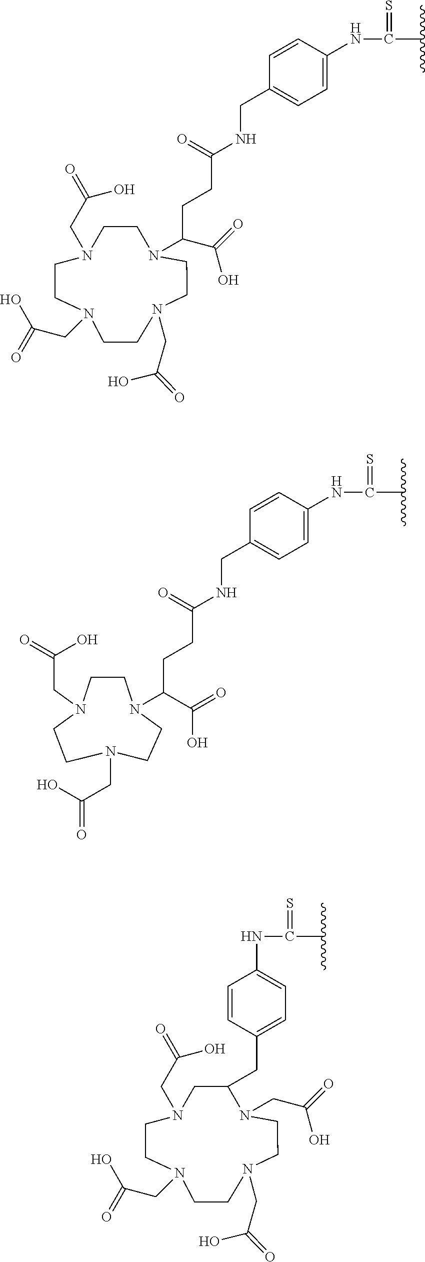 [SMILES: *C(=S)Nc1ccc(CNC(=O)CCC(C(=O)O)N2CCN(CC(=O)O)CCN(CC(=O)O)CCN(CC(=O)O)CC2)cc1 ; *C(=S)Nc1ccc(CNC(=O)CCC(C(=O)O)N2CCN(CC(=O)O)CCN(CC(=O)O)CC2)cc1 ; *C(=S)Nc1ccc(CC2CN(CC(=O)O)CCN(CC(=O)O)CCN(CC(=O)O)CCN2CC(=O)O)cc1]

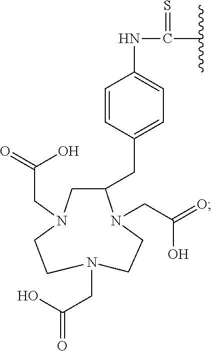 [SMILES: *C(=S)Nc1ccc(CC2CN(CC(=O)O)CCN(CC(=O)O)CCN2CC(=O)O)cc1]

X is present or absent, and X is selected from the group consisting of linking group 1 and linking group 2 when X is present;
linking group 1 is one selected from the group consisting of:

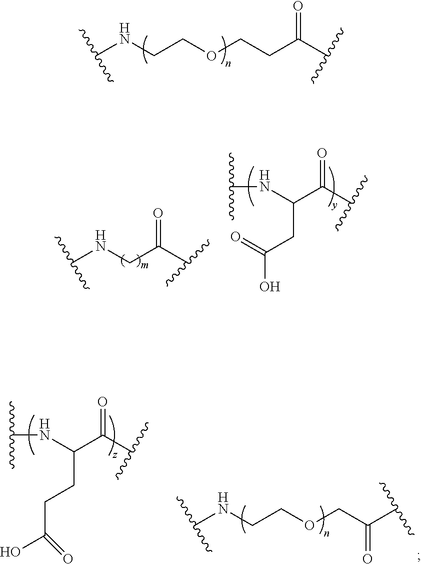

linking group 2 is one selected from the group consisting of:

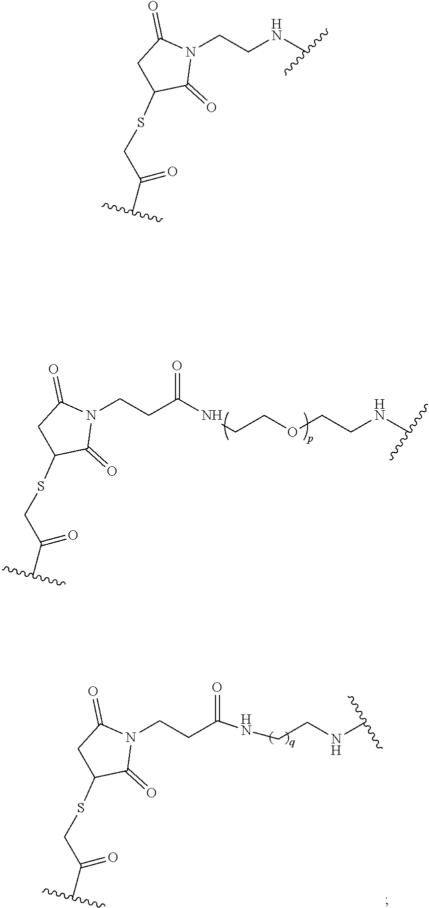

n, m, y, z, p, and q independently are an integer of 0 to 10.

The present disclosure further provides a preparation method of the Glu-urea compound, where
(i) when X is absent or is the linking group 1, the preparation method includes the following steps:
subjecting Polypeptide compound 1 to first substitution with an R₁ active compound to obtain Intermediate 1; and
subjecting Intermediate 1 to deprotection of an R₃ protecting group and then second substitution with an R₂ active compound in sequence to obtain the Glu-urea compound; where
one of the R₁ active compound and the R₂ active compound is selected from the group consisting of

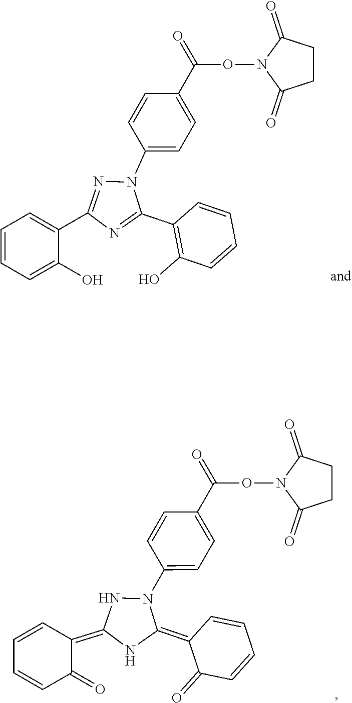 [SMILES: O=C(ON1C(=O)CCC1=O)c1ccc(-n2nc(-c3ccccc3O)nc2-c2ccccc2O)cc1 ; O=C1C=CC=C/C1=C1/N/C(=C2/C=CC=CC2=O)N(c2ccc(C(=O)ON3C(=O)CCC3=O)cc2)N1]

while the other is selected from the group consisting of:

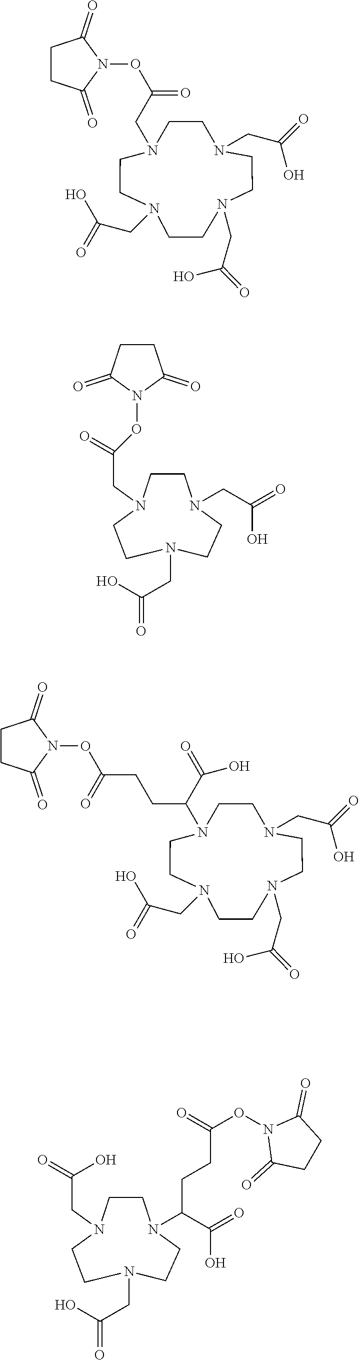 [SMILES: O=C(O)CN1CCN(CC(=O)O)CCN(CC(=O)ON2C(=O)CCC2=O)CCN(CC(=O)O)CC1 ; O=C(O)CN1CCN(CC(=O)O)CCN(CC(=O)ON2C(=O)CCC2=O)CC1 ; O=C(O)CN1CCN(CC(=O)O)CCN(C(CCC(=O)ON2C(=O)CCC2=O)C(=O)O)CCN(CC(=O)O)CC1 ; O=C(O)CN1CCN(CC(=O)O)CCN(C(CCC(=O)ON2C(=O)CCC2=O)C(=O)O)CC1]

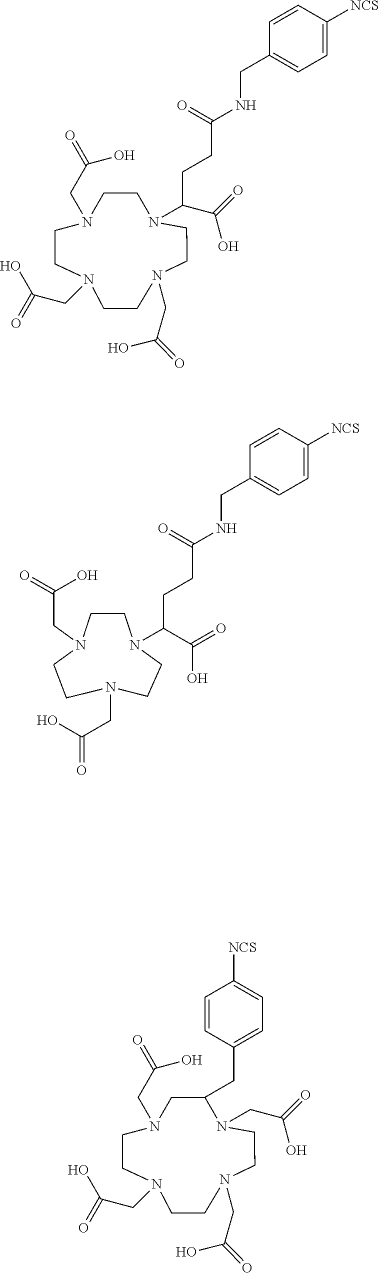

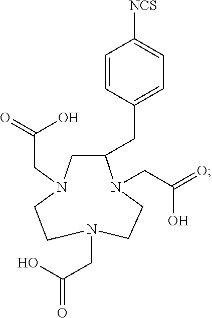

when Polypeptide compound 1 has the following structure:

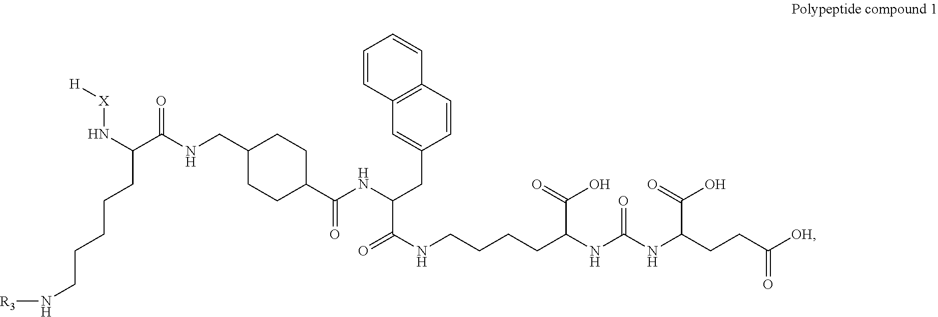

then Intermediate 1 has the following structure:

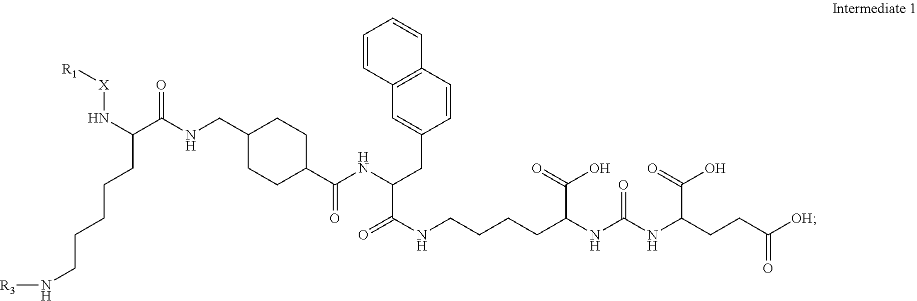

and
when Polypeptide compound 1 has the following structure:

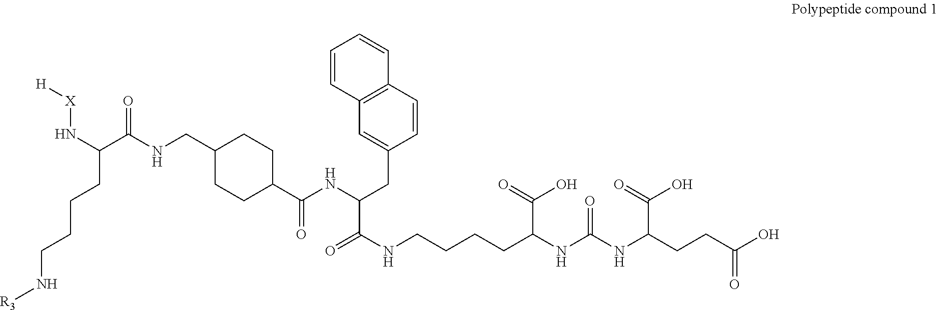

then Intermediate 1 has the following structure:

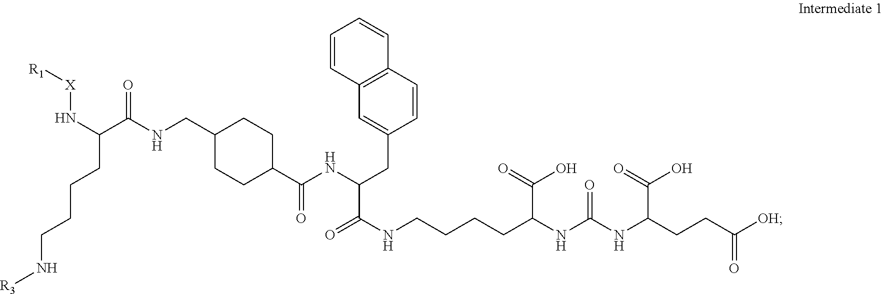

X and R₁ in Polypeptide compound 1 and Intermediate 1 are the same as X and R₁ in formula I-1 or formula I-2; and R₃ is selected from the group consisting of a Boc protecting group, a DDE protecting group, and an Fmoc protecting group; and
(ii) when X is linking group 2, the preparation method includes the following steps:
subjecting Polypeptide compound 2 to third substitution with R₁-L to obtain intermediate 2; and
subjecting Intermediate 2 to fourth substitution with an R₂ active compound to obtain the Glu-urea compound; where
L in R₁-L is one selected from the group consisting of:

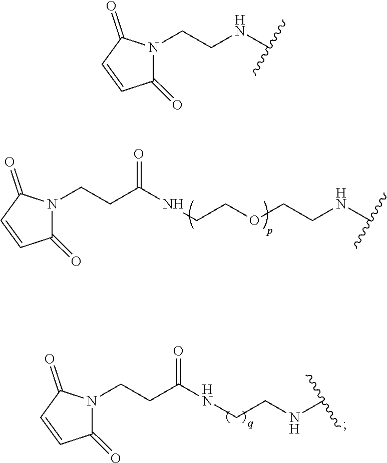

p and q in L are the same as those defined in linking group 2;
when R₁ in R₁-L is selected from the group consisting of

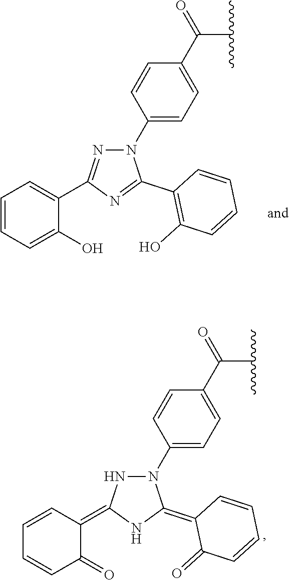 [SMILES: *C(=O)c1ccc(-n2nc(-c3ccccc3O)nc2-c2ccccc2O)cc1 ; *C(=O)c1ccc(N2N/C(=C3\C=CC=CC3=O)N/C2=C2/C=CC=CC2=O)cc1]

the R₂ active compound is one selected from the group consisting of:

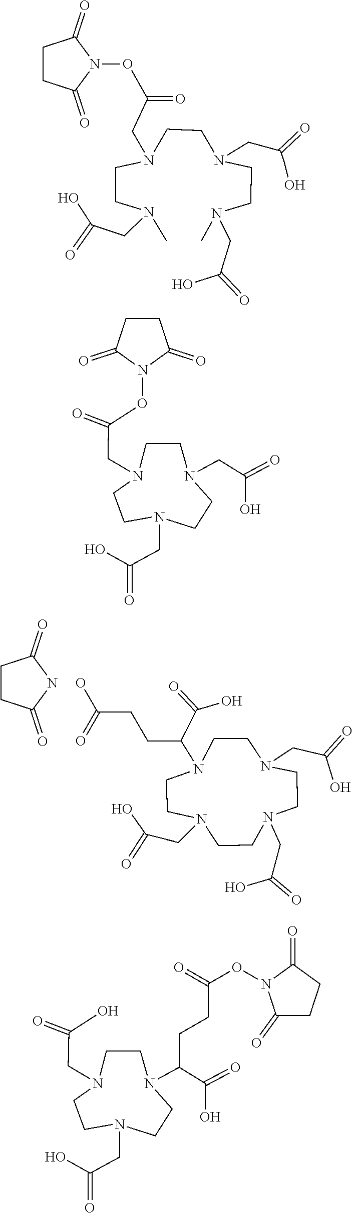

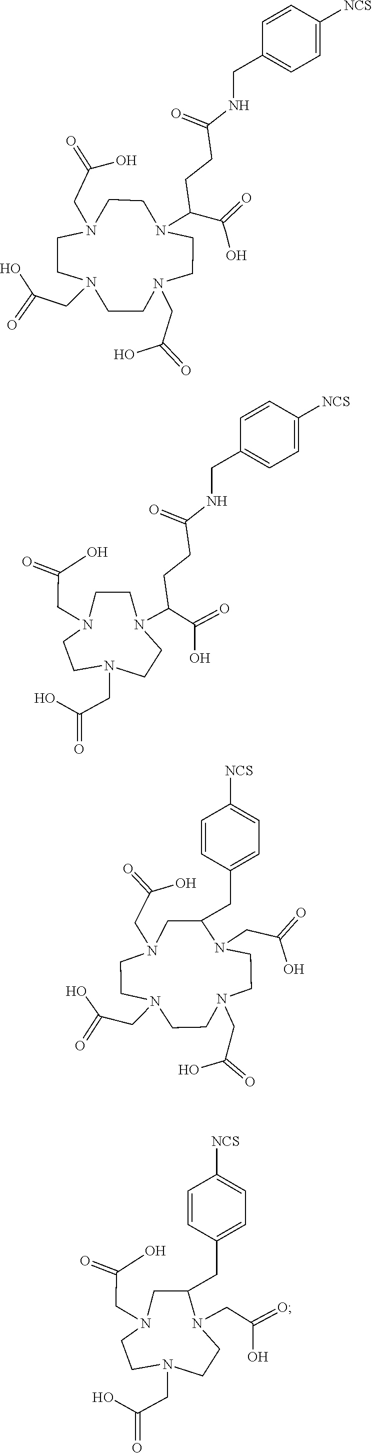

when R₁ in R₁-L is the group to be labeled, the R₂ active compound is selected from the group consisting of

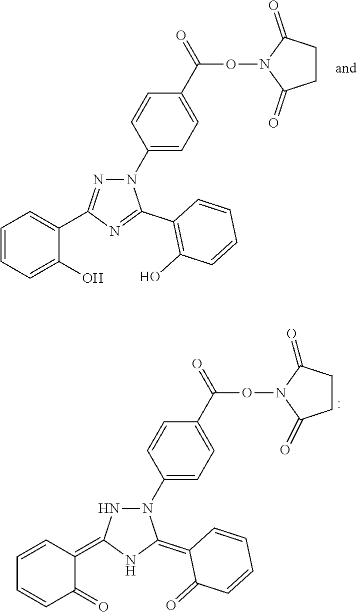 [SMILES: O=C(ON1C(=O)CCC1=O)c1ccc(-n2nc(-c3ccccc3O)nc2-c2ccccc2O)cc1 ; O=C1C=CC=C/C1=C1/N/C(=C2/C=CC=CC2=O)N(c2ccc(C(=O)ON3C(=O)CCC3=O)cc2)N1]

when Polypeptide compound 2 has the following structure:

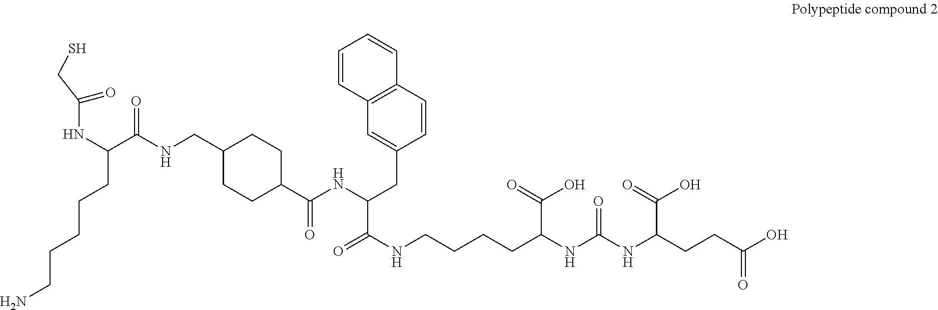 [SMILES: NCCCCCC(NC(=O)CS)C(=O)NCC1CCC(C(=O)NC(Cc2ccc3ccccc3c2)C(=O)NCCCCC(NC(=O)NC(CCC(=O)O)C(=O)O)C(=O)O)CC1]

then Intermediate 2 has the following structure:

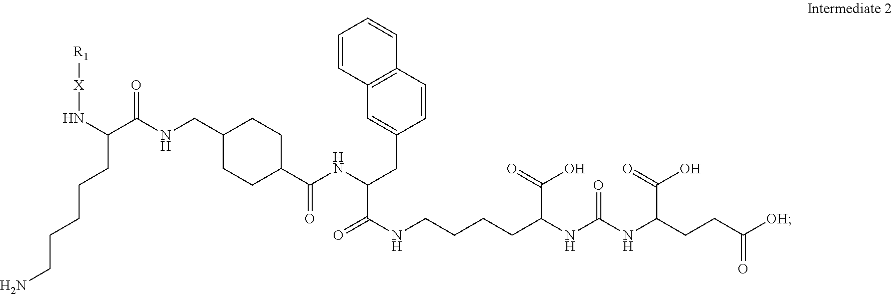

and
when Polypeptide compound 2 has the following structure:

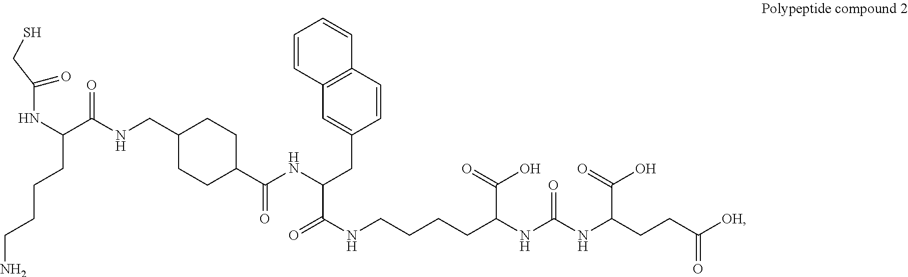 [SMILES: NCCCCC(NC(=O)CS)C(=O)NCC1CCC(C(=O)NC(Cc2ccc3ccccc3c2)C(=O)NCCCCC(NC(=O)NC(CCC(=O)O)C(=O)O)C(=O)O)CC1]

then Intermediate 2 has the following structure:

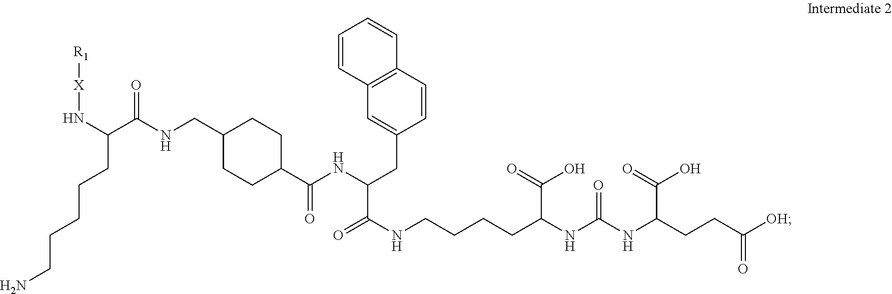

X and R₁ in Polypeptide compound 2 and Intermediate 2 are the same as X and R₁ in formula I-1 or formula I-2.

The present disclosure further provides a pharmaceutically acceptable salt of a Glu-urea compound, where the pharmaceutically acceptable salt is prepared by subjecting the Glu-urea compound to a reaction with an acid or an alkali; and the Glu-urea compound is the Glu-urea compound described above or a Glu-urea compound prepared by the preparation method.

The present disclosure further provides a nuclide-targeted probe, where the nuclide-targeted probe is prepared by subjecting the group to be labeled in the Glu-urea compound or the pharmaceutically acceptable salt of the Glu-urea compound to complexation with a labeling nuclide.

Preferably, the labeling nuclide is at least one selected from the group consisting of ¹⁸F, ⁴⁷Sc, ⁶⁴Cu, ⁶⁷Cu, ⁶⁷Ga, ⁶⁸Ga, ⁸⁹Zr, ⁸⁶Y, ⁸⁹Sr, ⁹⁰Y, ^{99m}Tc, ¹⁰⁵Rh, ¹⁰⁹Pd, ¹¹¹In, ¹¹⁹Sb, ¹⁴⁹Tb, ¹⁵³Sm, ¹⁵⁷Gd, ¹⁶¹Tb, ¹⁶⁶Ho, ¹⁷⁷Lu, ¹⁸⁶Re, ¹⁸⁸Re, ²⁰¹Tl, ²⁰³Pb, ²¹²Pb, ²¹²Bi, ²¹³Bi, ²²³Ra, ²²⁷Th, and ²²⁵Ac.

The present disclosure further provides a preparation method of the nuclide-targeted probe, including the following steps: subjecting the Glu-urea compound or the pharmaceutically acceptable salt thereof to complexation with the labeling nuclide to obtain the nuclide-targeted probe or a pharmaceutically acceptable salt of the nuclide-targeted probe; where the Glu-urea compound or the pharmaceutically acceptable salt thereof is the Glu-urea compound described above, or a Glu-urea compound prepared by the preparation method, or the pharmaceutically acceptable salt of the Glu-urea compound.

The present disclosure further provides a pharmaceutically acceptable salt of a nuclide-targeted probe, where the pharmaceutically acceptable salt of the nuclide-targeted probe is prepared by subjecting the nuclide-targeted probe or a Glu-urea compound to a salt-forming reaction; the nuclide-targeted probe is the nuclide-targeted probe described above or a nuclide-targeted probe prepared by the preparation method; and the Glu-urea compound is the Glu-urea compound described above or a Glu-urea compound prepared by the preparation method.

The present disclosure further provides a pharmaceutical composition, including an active component and a pharmaceutically acceptable auxiliary material; where the active component is one or more selected from the group consisting of a Glu-urea compound, a nuclide-targeted probe, the pharmaceutically acceptable salt of the Glu-urea compound, and the pharmaceutically acceptable salt of the nuclide-targeted probe; the Glu-urea compound is the Glu-urea compound described above or a Glu-urea compound prepared by the preparation method; and the nuclide-targeted probe is the nuclide-targeted probe described above or a nuclide-targeted probe prepared by the preparation method.

The present disclosure further provides use of the Glu-urea compound, a Glu-urea compound prepared by the preparation method, the pharmaceutically acceptable salt of the Glu-urea compound, the nuclide-targeted probe, a nuclide-targeted probe prepared by the preparation method, the pharmaceutically acceptable salt of the nuclide-targeted probe, or the pharmaceutical composition in preparation of a therapeutic drug or a diagnostic drug for a prostate-specific membrane antigen (PSMA) protein-mediated disease.

Preferably, the PSMA protein-mediated disease includes a tumor.

In the present disclosure, the Glu-urea compound is a DFX-modified compound. DFX has a certain ability to target tumor lesions, and has been approved as an iron chelator for clinical use with high safety. Modifying a DFX structure into the PSMA-targeted probe has significantly improved an uptake of the probe at the target site and prolonged a residence time of the probe at the target site. The Glu-urea compound can be used as a label for a variety of diagnostic and therapeutic nuclides, and can also be used to construct an imaging treatment platform based on pairs of diagnostic and therapeutic nuclides, thereby achieving excellent application prospects in the preparation of therapeutic drug or diagnostic drug for PSMA protein-mediated diseases.

In the present disclosure, the Glu-urea compound can chelate with the nuclide through the group to be labeled to form a PSMA-targeted nuclide probe with high affinity and specificity. This type of probe shows strong labeling ability, short labeling time, and high labeling yield, which are beneficial to the commercial application and clinical promotion of nuclide-targeted probes. Compared with existing PSMA-targeted probes, the nuclide-targeted probe has suitable metabolic kinetic properties (different pharmacokinetic properties) and high lesion uptake and retention time. The nuclide-targeted probe shows excellent diagnostic and therapeutic effects on PSMA protein-mediated diseases, and is a nuclide-targeted diagnostic and therapeutic drug with great application prospects. As shown in the test results of the examples, the ¹⁷⁷Lu-labeled probe has an uptake absolute value in tumors that is 4 to 5 times that of [¹⁷⁷Lu]Lu-PSMA617 (one of the current gold standards), and is a nuclide-targeted therapeutic drug with great application prospects. This probe overcomes the shortcomings of existing small-molecule PSMAs such as rapid metabolism and short target organ retention time, improves the effect of PSMA nuclide-targeted therapy, and has a potential to be widely used in clinical applications. In addition to treatment, the nuclide-targeted probe is distributed in the organism, forming a concentration difference. The rays emitted by the probe or their changes in magnetoresistance are detected by external instruments; and then an image is formed after reconstruction, and can provide diagnostic information for the disease and achieve the effect of integrated diagnosis and treatment. In addition, by adjusting the appropriate specific activity or drug combination, it is helpful to obtain a better target/non-target ratio and enhance the uptake of nuclide-targeted probes in tumors.

### BRIEF DESCRIPTION OF THE DRAWINGS

FIG. 1 shows a mass spectrum for identification of compound PKND01;

FIG. 2 shows a mass spectrum for identification of compound PKND02;

FIG. 3 shows a mass spectrum for identification of compound PKSD01;

FIG. 4 shows a mass spectrum for identification of compound PKSD02;

FIG. 5 shows a mass spectrum for identification of compound PKSP₂D01;

FIG. 6 shows a mass spectrum for identification of compound Gd-PKND01;

FIG. 7A-FIG. 7E show high-performance liquid chromatography (HPLC) for identification patterns of compounds PKND01 (FIG. 7A), PKND02 (FIG. 7B), PKSD01 (FIG. 7C), PKSD02 (FIG. 7D), and PKSP2D01 (FIG. 7E);

FIG. 8 shows an HPLC identification pattern of probe Gd-PKND01;

FIG. 9A-FIG. 9D show HPLC identification patterns of radiochemical purity of probes [⁶⁸Ga]Ga-PKND01 (FIG. 9A), [⁶⁸Ga]Ga-PKND02 (FIG. 9B), [⁶⁸Ga]Ga-PKSD01 (FIG. 9C), and [⁶⁸Ga]Ga-PKSP2D01 (FIG. 9D);

FIG. 10A-FIG. 10C show HPLC identification patterns of radiochemical purity of probes [¹⁷⁷Lu]Lu-PKND01 (FIG. 10A), [¹⁷⁷Lu] Lu-PKND02 (FIG. 10B), [¹⁷⁷Lu]Lu-PKSD01 (FIG. 10C), and [¹⁷⁷Lu]Lu-PKE₃D01 (FIG. 10D);

FIG. 11A-FIG. 11C show HPLC identification results of a stability of probes [⁶⁸Ga]Ga-PKND01 (FIG. 11A), [⁶⁸Ga]Ga-PKND02 (FIG. 11B), and [⁶⁸Ga]Ga-PKSD01 (FIG. 11C);

FIG. 12A-FIG. 12C show HPLC identification results of a stability of probes [¹⁷⁷Lu]Lu-PKND01 (FIG. 12A), [¹⁷⁷Lu]Lu-PKND02 (FIG. 12B), and [¹⁷⁷Lu]Lu-PKSD01 (FIG. 12C);

FIG. 13A-FIG. 13B show cellular uptake and inhibition results of probes [¹⁷⁷Lu]Lu-PKND01 (FIG. 13A) and [¹⁷⁷Lu]Lu-PKND02 (FIG. 13B);

FIG. 14A-FIG. 14B show cellular uptake and inhibition results of probes [¹⁷⁷Lu]Lu-PKSD01 (FIG. 14A) and [¹⁷⁷Lu]Lu-PKSD02 (FIG. 14B);

FIG. 15A-FIG. 15B show a PET imaging result (FIG. 15A) and an uptake quantitative value of the tissue of interest (FIG. 15B) for [⁶⁸Ga]Ga-PKND01;

FIG. 16A-FIG. 16B show a PET imaging result (FIG. 16A) and an uptake quantitative value of the tissue of interest (FIG. 16B) for [⁶⁸Ga]Ga-PKND02;

FIG. 17A-FIG. 17B show a PET imaging result (FIG. 17A) and an uptake quantitative value of the tissue of interest (FIG. 17B) for [⁶⁸Ga]Ga-PKSD01;

FIG. 18A-FIG. 18B show a PET imaging result (FIG. 18A) and an uptake quantitative value of the tissue of interest (FIG. 18B) for [⁶⁸Ga]Ga-PKSP₂D01;

FIG. 19A-FIG. 19B show a SPECT imaging result (FIG. 19A) and a target/non-target ratio (FIG. 19B) of [¹⁷⁷Lu]Lu-PKND01;

FIG. 20A-FIG. 20B show a SPECT imaging result (FIG. 20A) and a target/non-target ratio (FIG. 20B) of [¹⁷⁷Lu]Lu-PKND02;

FIG. 21A-FIG. 21B show a SPECT imaging result (FIG. 21A) and a target/non-target ratio (FIG. 21B) of [¹⁷⁷Lu]Lu-PKSD01;

FIG. 22A-FIG. 22B show a SPECT imaging result (FIG. 22A) of [¹⁷⁷Lu]Lu-PSMA617, as well as a ratio (FIG. 22B) of the uptake count of [¹⁷⁷Lu]Lu-PKND01 and [¹⁷⁷Lu]Lu-PKSD01 at the tumor site to [¹⁷⁷Lu]Lu-PSMA617;

FIG. 23A-FIG. 23B show a SPECT imaging tumor uptake result (FIG. 23A) and a tumor/kidney ratio (FIG. 23B) of [¹⁷⁷Lu]Lu-PKND01 with different specific activities;

FIG. 24 shows MRI results at different time points before and after tail vein injection of Gd-PKND01 in tumor-bearing mice;

FIG. 25A-FIG. 25B show a biodistribution result (FIG. 25A) and a tumor/kidney ratio at different time points (FIG. 25B) of [¹⁷⁷Lu]Lu-PKND01 in tumor-bearing mice;

FIG. 26A-FIG. 26B show a biodistribution result of [¹⁷⁷Lu]Lu-PSMA617 in tumor-bearing mice (FIG. 26A) and a comparison of the uptake value of the [¹⁷⁷Lu]Lu-PSMA617 in tumors with that of [¹⁷⁷Lu]Lu-PKND01 (FIG. 26B);

FIG. 27 shows treatment results of [¹⁷⁷Lu]Lu-PKND01, [¹⁷⁷Lu]Lu-PKSD01, and [¹⁷⁷Lu]Lu-PSMA617 in tumor-bearing mice;

FIG. 28 shows a mass spectrum for identification of compound PK2ND01;

FIG. 29 shows a mass spectrum for identification of compound PK2NGD01;

FIG. 30 shows a mass spectrum for identification of compound PKED01;

FIG. 31 shows a mass spectrum for identification of compound PKE₃D01;

FIG. 32 shows a mass spectrum for identification of compound PKP₂₂D01;

FIG. 33A-FIG. 33B show HPLC identification patterns of radiochemical purity of probes [⁶⁸Ga]Ga-PK2ND01 (FIG. 33A) and [⁶⁸Ga]Ga-PK2NGD01 (FIG. 33B);

FIG. 34A-FIG. 34C show HPLC identification patterns of radiochemical purity of probes [⁶⁸Ga]Ga-PKED01 (FIG. 34A), [⁶⁸Ga]Ga-PKE₃D01 (FIG. 34B), and [⁶⁸Ga]Ga-PKP₂₂D01 (FIG. 34C);

FIG. 35A-FIG. 35B show cellular uptake and inhibition results (FIG. 35A) and cell internalization and membrane uptake rates (FIG. 35B) of probe [¹⁷⁷Lu]Lu-PKE₃D01;

FIG. 36A-FIG. 36B show a PET imaging result (FIG. 36A) and an uptake quantitative value of the tissue of interest (FIG. 36B) for [⁶⁸Ga]Ga-PK2ND01;

FIG. 37A-FIG. 37B show a PET imaging result (FIG. 37A) and an uptake quantitative value of the tissue of interest (FIG. 37B) for [⁶⁸Ga]Ga-PK2NGD01;

FIG. 38A-FIG. 38B show a PET imaging result (FIG. 38A) and an uptake quantitative value of the tissue of interest (FIG. 38B) for [⁶⁸Ga]Ga-PKED01;

FIG. 39A-FIG. 39B show a PET imaging result (FIG. 39A) and an uptake quantitative value of the tissue of interest (FIG. 39B) for [⁶⁸Ga]Ga-PKE₃D01; and

FIG. 40A-FIG. 40B show a PET imaging result (FIG. 40A) and an uptake quantitative value of the tissue of interest (FIG. 40B) for [⁶⁸Ga]Ga-PKP₂₂D01.

### DETAILED DESCRIPTION OF THE EMBODIMENTS

The present disclosure provides a glutamic acid (Glu)-urea compound, where the Glu-urea compound has a structure shown in formula I-1 or formula I-2:

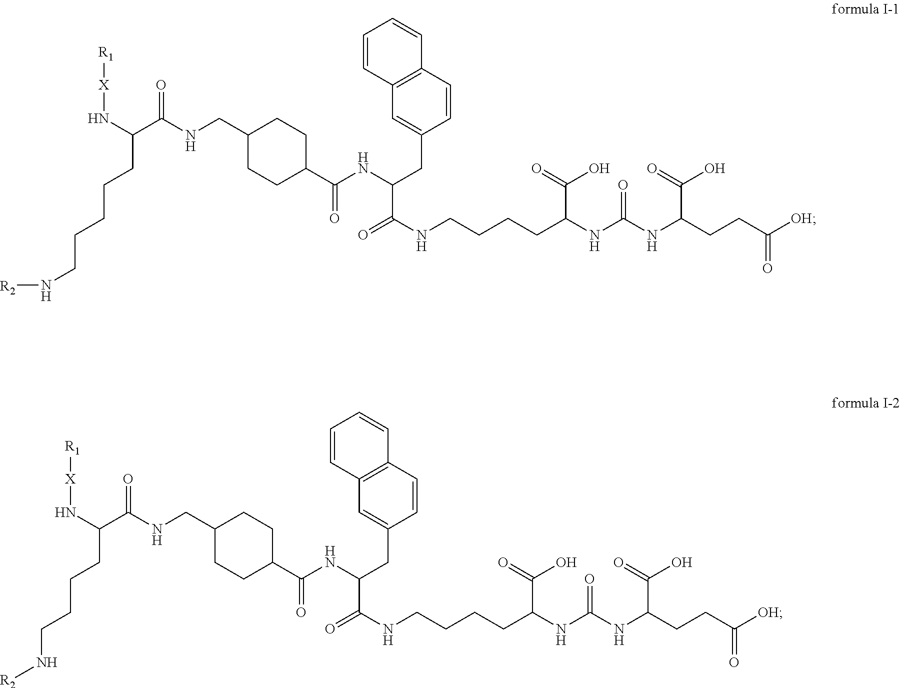

wherein one of R₁ and R₂ is selected from the group consisting of

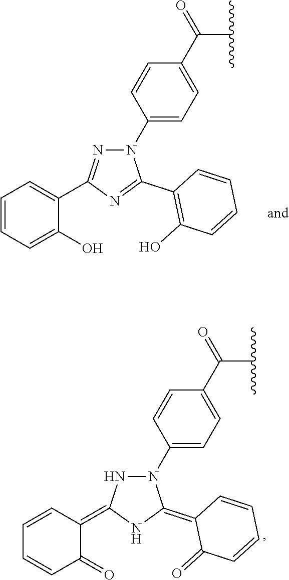 [SMILES: *C(=O)c1ccc(-n2nc(-c3ccccc3O)nc2-c2ccccc2O)cc1 ; *C(=O)c1ccc(N2N/C(=C3\C=CC=CC3=O)N/C2=C2/C=CC=CC2=O)cc1]

while the other is a group to be labeled, and the group to be labeled is one selected from the group consisting of:

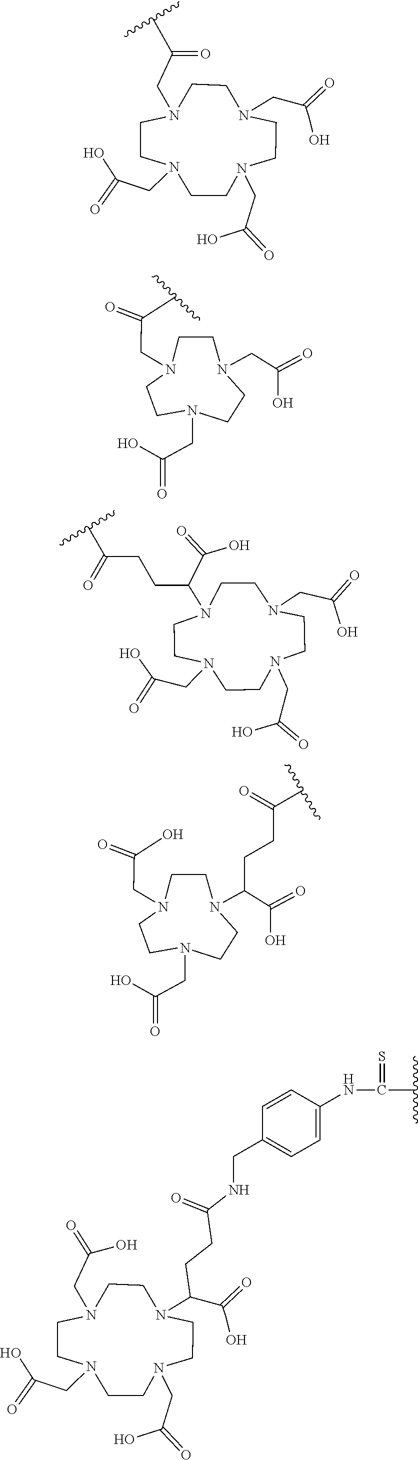

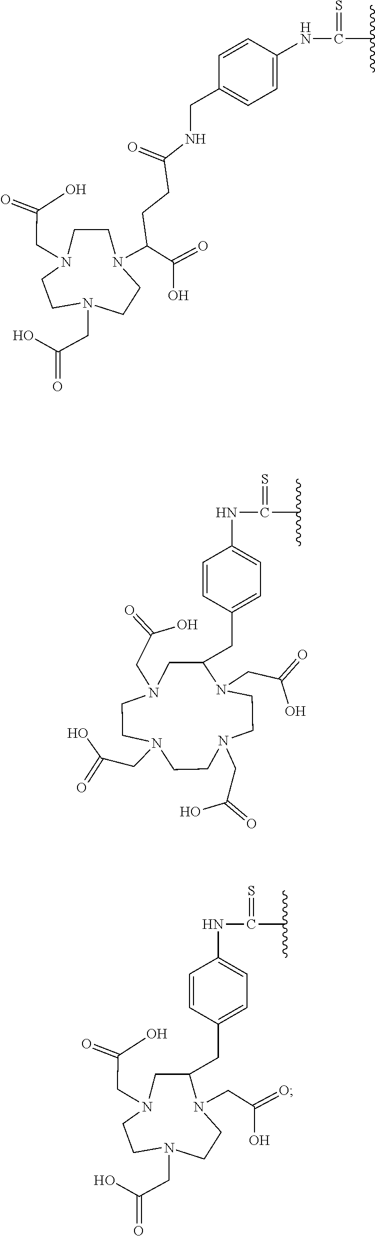 [SMILES: *C(=S)Nc1ccc(CNC(=O)CCC(C(=O)O)N2CCN(CC(=O)O)CCN(CC(=O)O)CC2)cc1 ; *C(=S)Nc1ccc(CC2CN(CC(=O)O)CCN(CC(=O)O)CCN(CC(=O)O)CCN2CC(=O)O)cc1 ; *C(=S)Nc1ccc(CC2CN(CC(=O)O)CCN(CC(=O)O)CCN2CC(=O)O)cc1]

X is present or absent, and X is selected from the group consisting of linking group 1 and linking group 2 when X is present;
linking group 1 is one selected from the group consisting of:

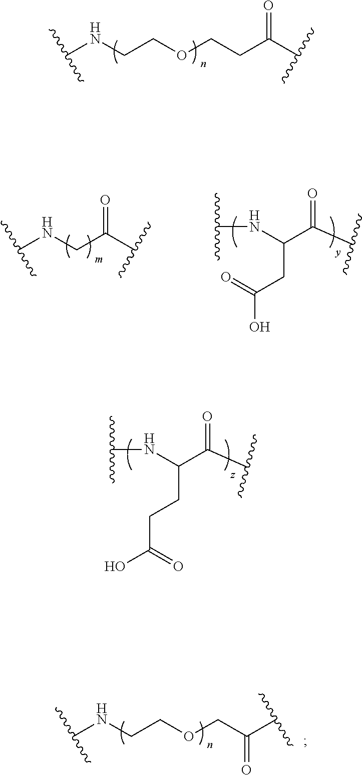

linking group 2 is one selected from the group consisting of:

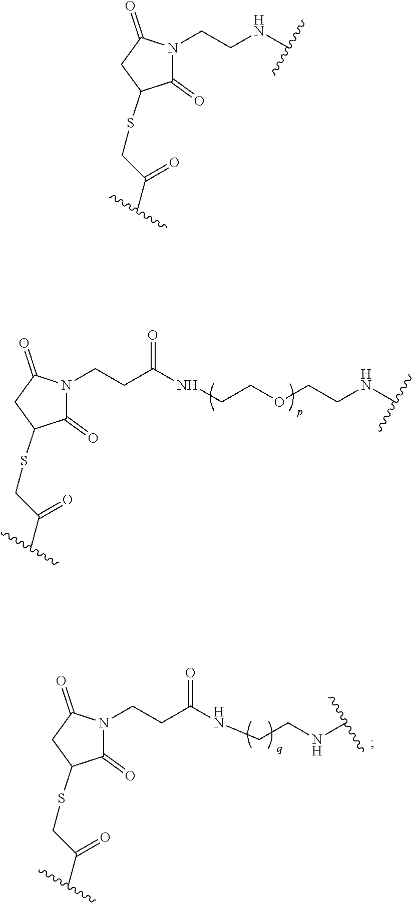

n, m, y, z, p, and q independently are an integer of 0 to 10.

In the present disclosure, when X is absent, one of R₁ and R₂ is

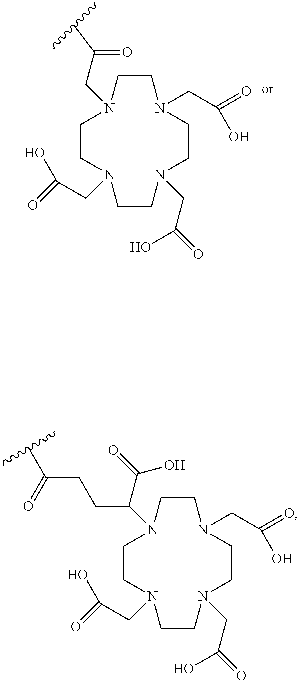 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

while the other is

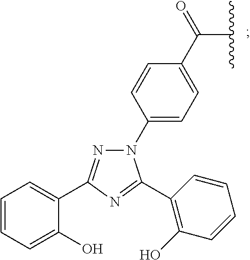 [SMILES: *C(=O)c1ccc(-n2nc(-c3ccccc3O)nc2-c2ccccc2O)cc1]

the Glu-urea compound includes PKND01, PKND02, PK2ND01, or PK2NGD01:

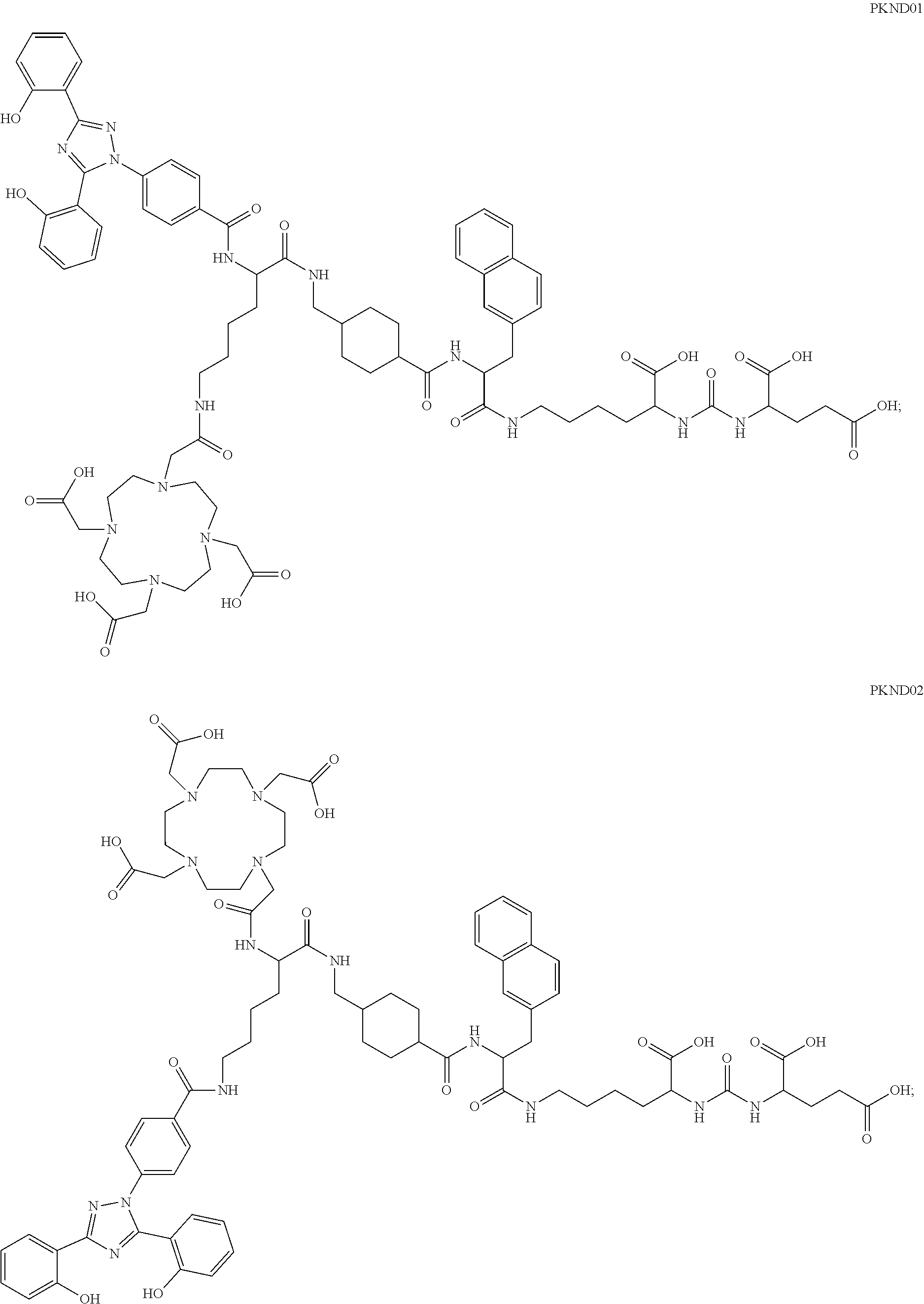 [SMILES: O=C(O)CCC(NC(=O)NC(CCCCNC(=O)C(Cc1ccc2ccccc2c1)NC(=O)C1CCC(CNC(=O)C(CCCCNC(=O)CN2CCN(CC(=O)O)CCN(CC(=O)O)CCN(CC(=O)O)CC2)NC(=O)c2ccc(-n3nc(-c4ccccc4O)nc3-c3ccccc3O)cc2)CC1)C(=O)O)C(=O)O ; O=C(O)CCC(NC(=O)NC(CCCCNC(=O)C(Cc1ccc2ccccc2c1)NC(=O)C1CCC(CNC(=O)C(CCCCNC(=O)c2ccc(-n3nc(-c4ccccc4O)nc3-c3ccccc3O)cc2)NC(=O)CN2CCN(CC(=O)O)CCN(CC(=O)O)CCN(CC(=O)O)CC2)CC1)C(=O)O)C(=O)O]

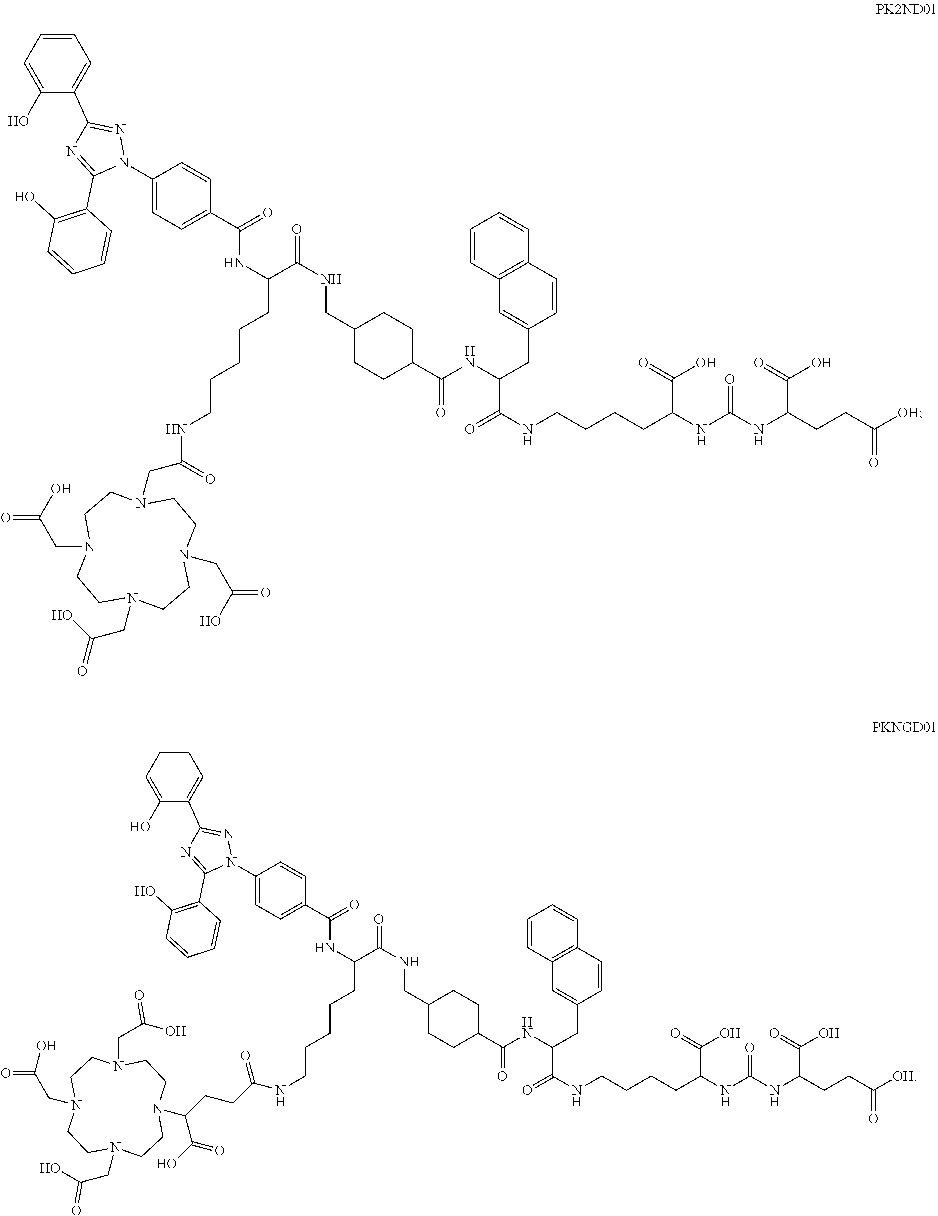 [SMILES: O=C(O)CCC(NC(=O)NC(CCCCNC(=O)C(Cc1ccc2ccccc2c1)NC(=O)C1CCC(CNC(=O)C(CCCCCNC(=O)CN2CCN(CC(=O)O)CCN(CC(=O)O)CCN(CC(=O)O)CC2)NC(=O)c2ccc(-n3nc(-c4ccccc4O)nc3-c3ccccc3O)cc2)CC1)C(=O)O)C(=O)O ; O=C(O)CCC(NC(=O)NC(CCCCNC(=O)C(Cc1ccc2ccccc2c1)NC(=O)C1CCC(CNC(=O)C(CCCCCNC(=O)CCC(C(=O)O)N2CCN(CC(=O)O)CCN(CC(=O)O)CCN(CC(=O)O)CC2)NC(=O)c2ccc(-n3nc(C4=CCCC=C4O)nc3-c3ccccc3O)cc2)CC1)C(=O)O)C(=O)O]

In the present disclosure, when X is

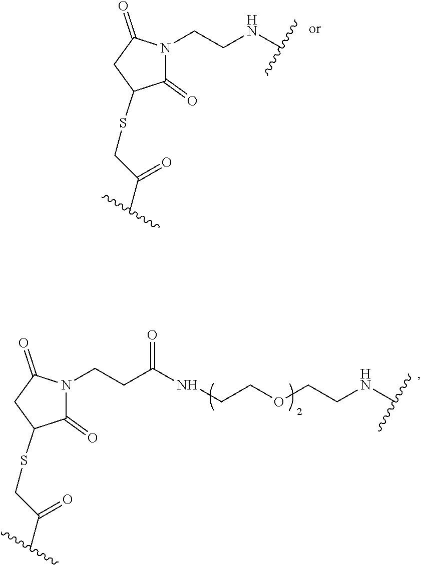

one of R₁ and R₂ is

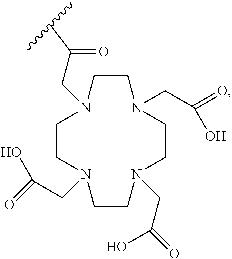 [SMILES: *C(=O)CN1CCN(CC(=O)O)CCN(CC(=O)O)CCN(CC(=O)O)CC1]

while the other is

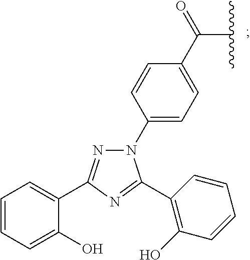 [SMILES: *C(=O)c1ccc(-n2nc(-c3ccccc3O)nc2-c2ccccc2O)cc1]

the Glu-urea compound includes PKSD01, PKSD02, PKSP₂D01, or PKSP₂D02:

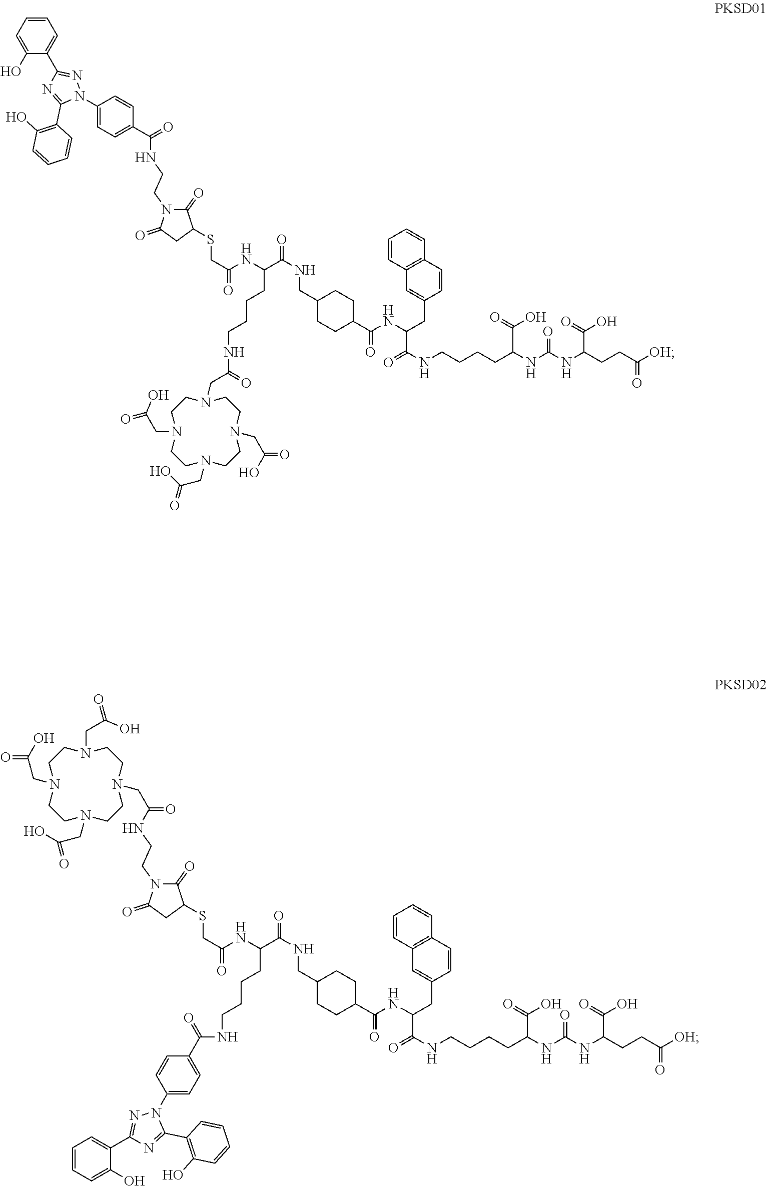 [SMILES: O=C(O)CCC(NC(=O)NC(CCCCNC(=O)C(Cc1ccc2ccccc2c1)NC(=O)C1CCC(CNC(=O)C(CCCCNC(=O)CN2CCN(CC(=O)O)CCN(CC(=O)O)CCN(CC(=O)O)CC2)NC(=O)CSC2CC(=O)N(CCNC(=O)c3ccc(-n4nc(-c5ccccc5O)nc4-c4ccccc4O)cc3)C2=O)CC1)C(=O)O)C(=O)O ; O=C(O)CCC(NC(=O)NC(CCCCNC(=O)C(Cc1ccc2ccccc2c1)NC(=O)C1CCC(CNC(=O)C(CCCCNC(=O)c2ccc(-n3nc(-c4ccccc4O)nc3-c3ccccc3O)cc2)NC(=O)CSC2CC(=O)N(CCNC(=O)CN3CCN(CC(=O)O)CCN(CC(=O)O)CCN(CC(=O)O)CC3)C2=O)CC1)C(=O)O)C(=O)O]

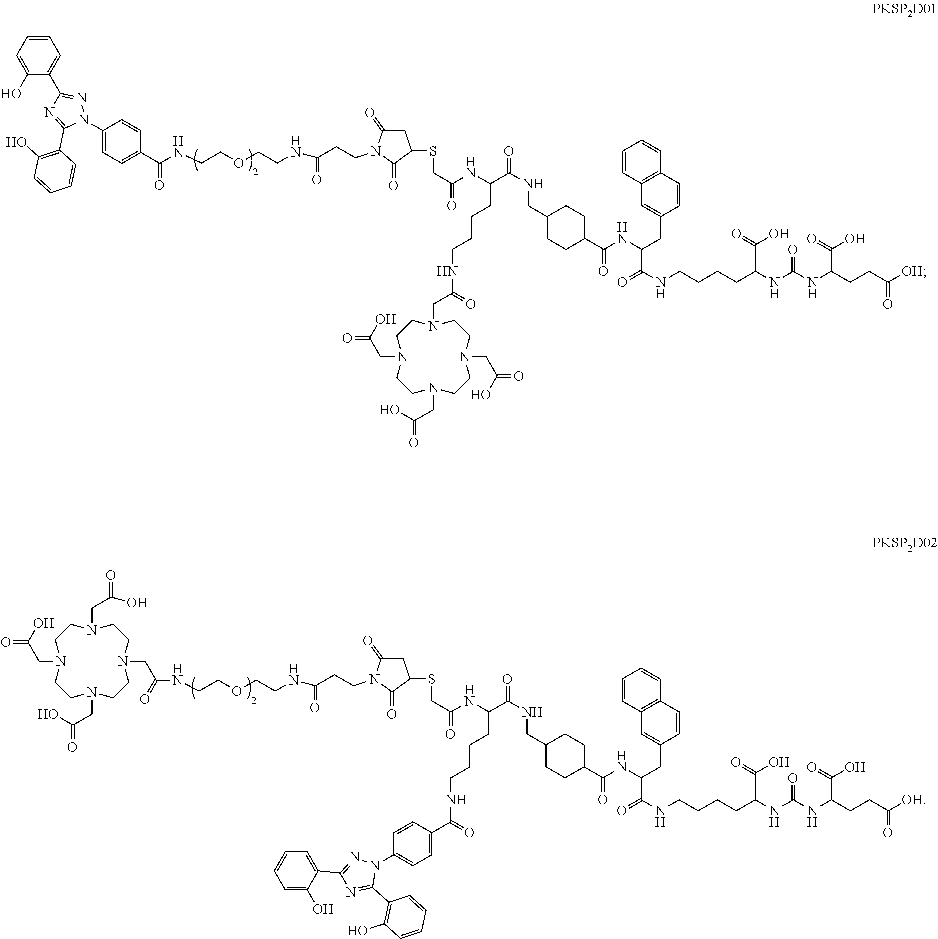

In the present disclosure, when X is

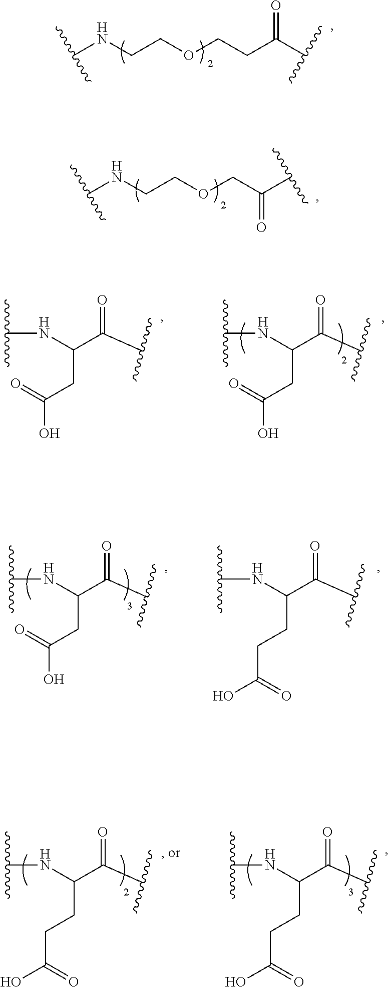

one of R₁ and R₂ is

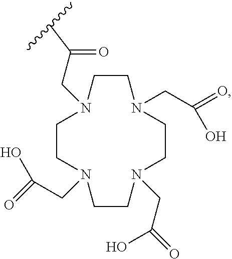 [SMILES: *C(=O)CN1CCN(CC(=O)O)CCN(CC(=O)O)CCN(CC(=O)O)CC1]

while the other is

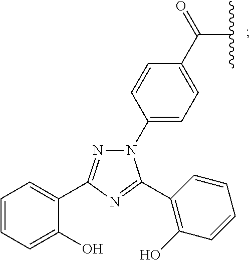 [SMILES: *C(=O)c1ccc(-n2nc(-c3ccccc3O)nc2-c2ccccc2O)cc1]

the Glu-urea compound includes PKP₂₃D01, PKP₂₃D02, PKP₂₂D01, PKP₂₂D02, PKED01, PKED02, PKDD01, PKDD02, PKD₂D01, PKD₃D01, PKE₂D01, or PKE₃D01:

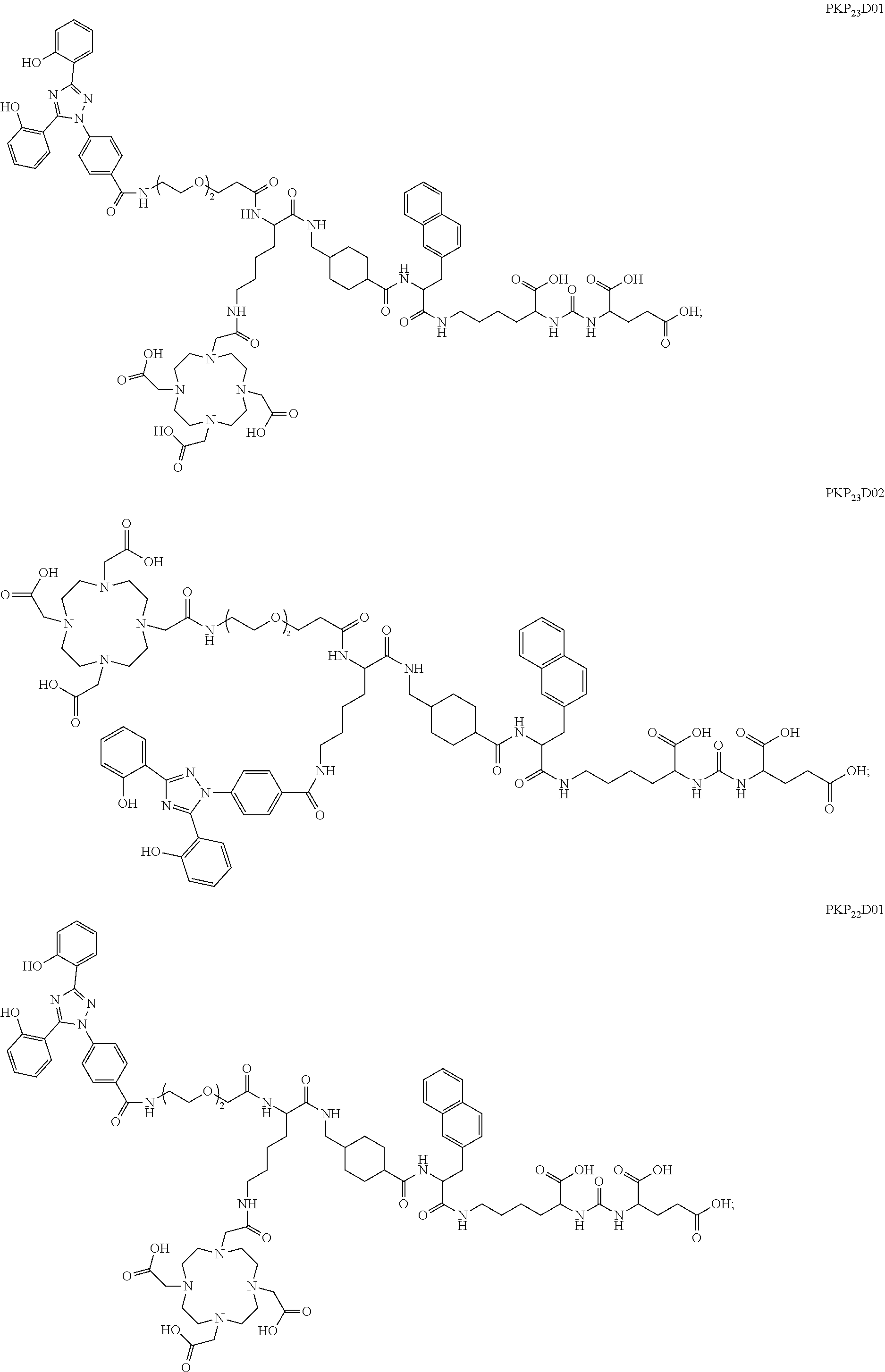

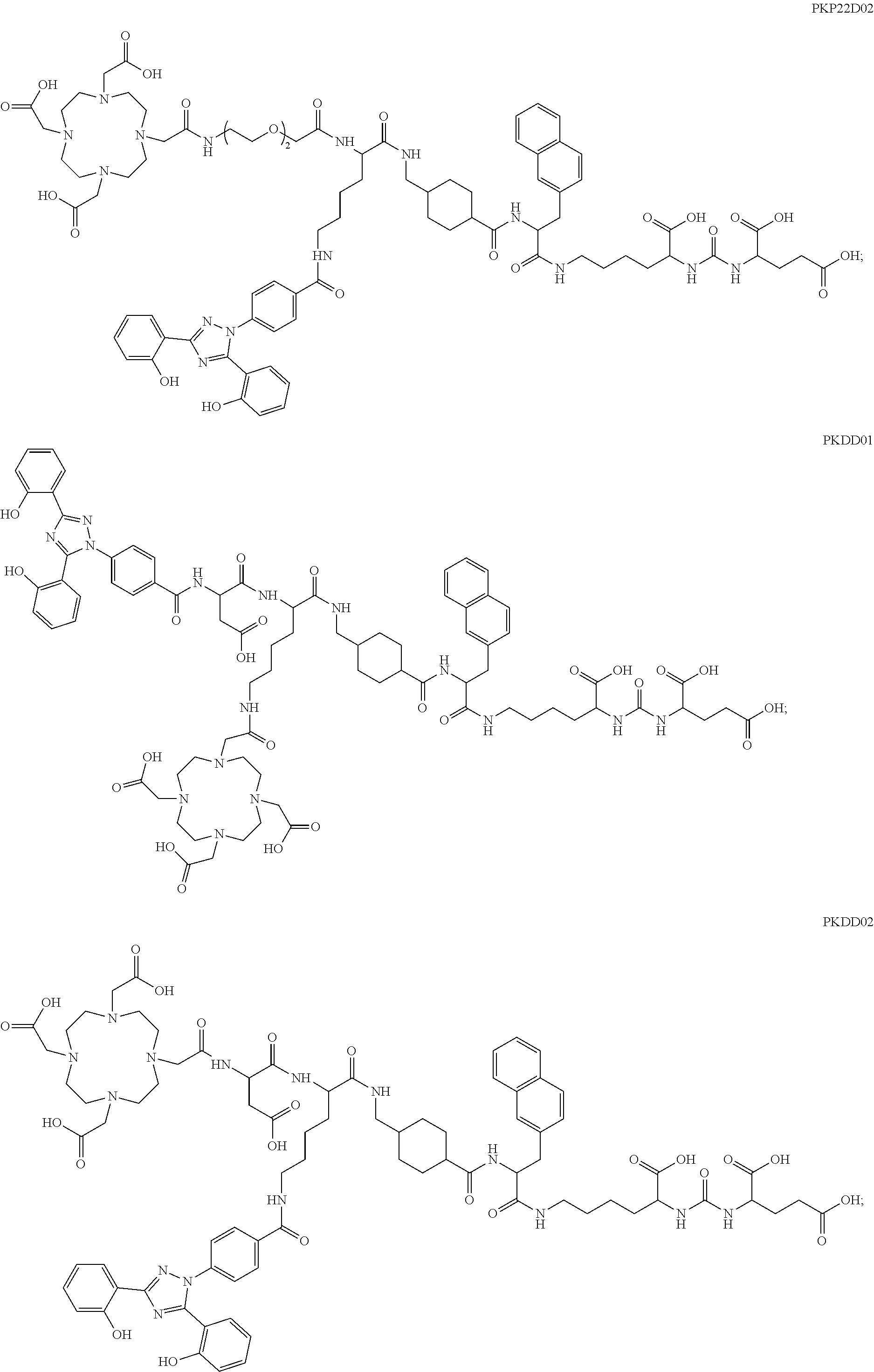

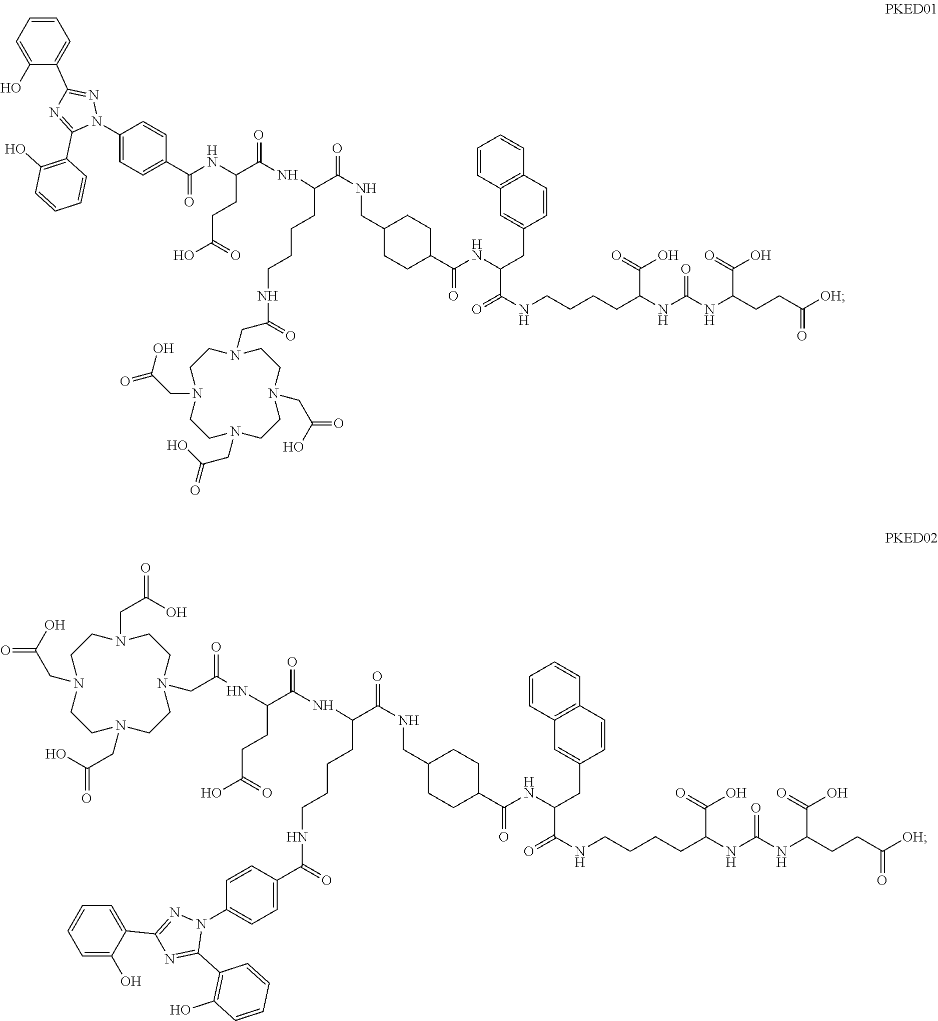 [SMILES: O=C(O)CCC(NC(=O)NC(CCCCNC(=O)C(Cc1ccc2ccccc2c1)NC(=O)C1CCC(CNC(=O)C(CCCCNC(=O)CN2CCN(CC(=O)O)CCN(CC(=O)O)CCN(CC(=O)O)CC2)NC(=O)C(CCC(=O)O)NC(=O)c2ccc(-n3nc(-c4ccccc4O)nc3-c3ccccc3O)cc2)CC1)C(=O)O)C(=O)O ; O=C(O)CCC(NC(=O)NC(CCCCNC(=O)C(Cc1ccc2ccccc2c1)NC(=O)C1CCC(CNC(=O)C(CCCCNC(=O)c2ccc(-n3nc(-c4ccccc4O)nc3-c3ccccc3O)cc2)NC(=O)C(CCC(=O)O)NC(=O)CN2CCN(CC(=O)O)CCN(CC(=O)O)CCN(CC(=O)O)CC2)CC1)C(=O)O)C(=O)O]

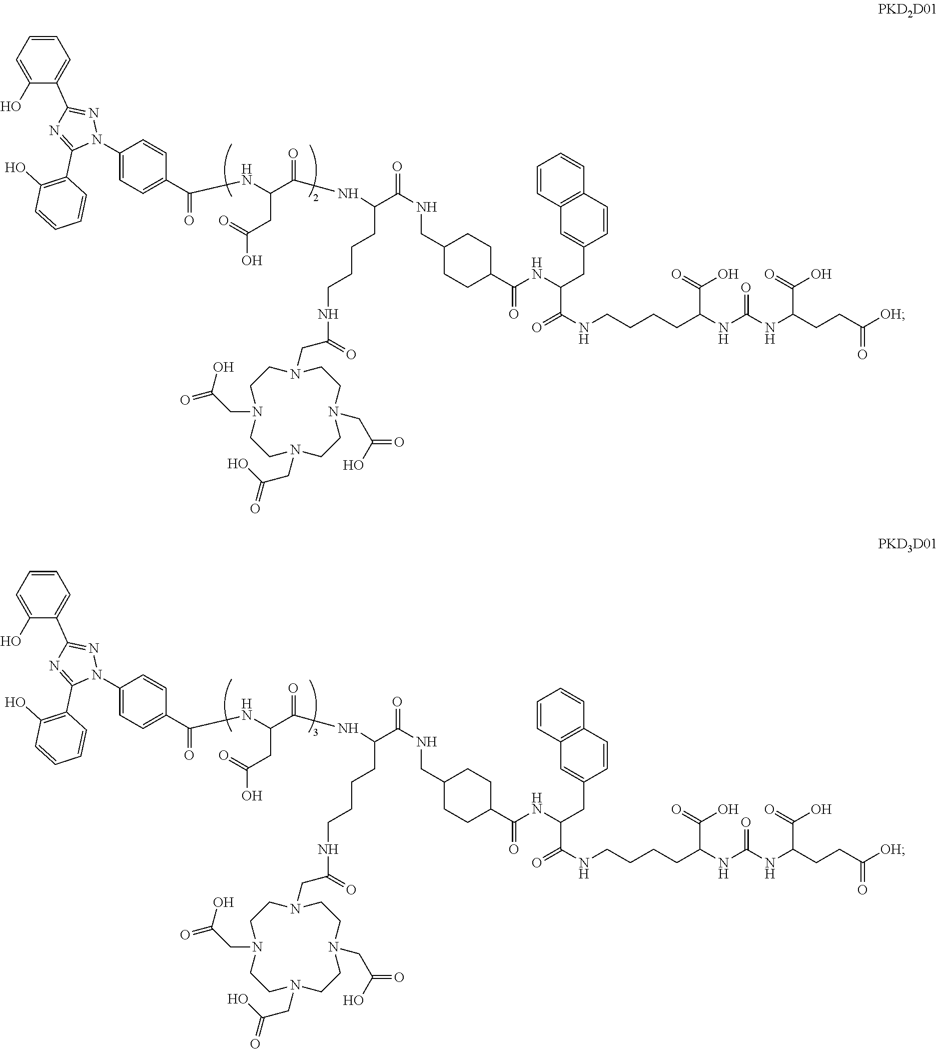

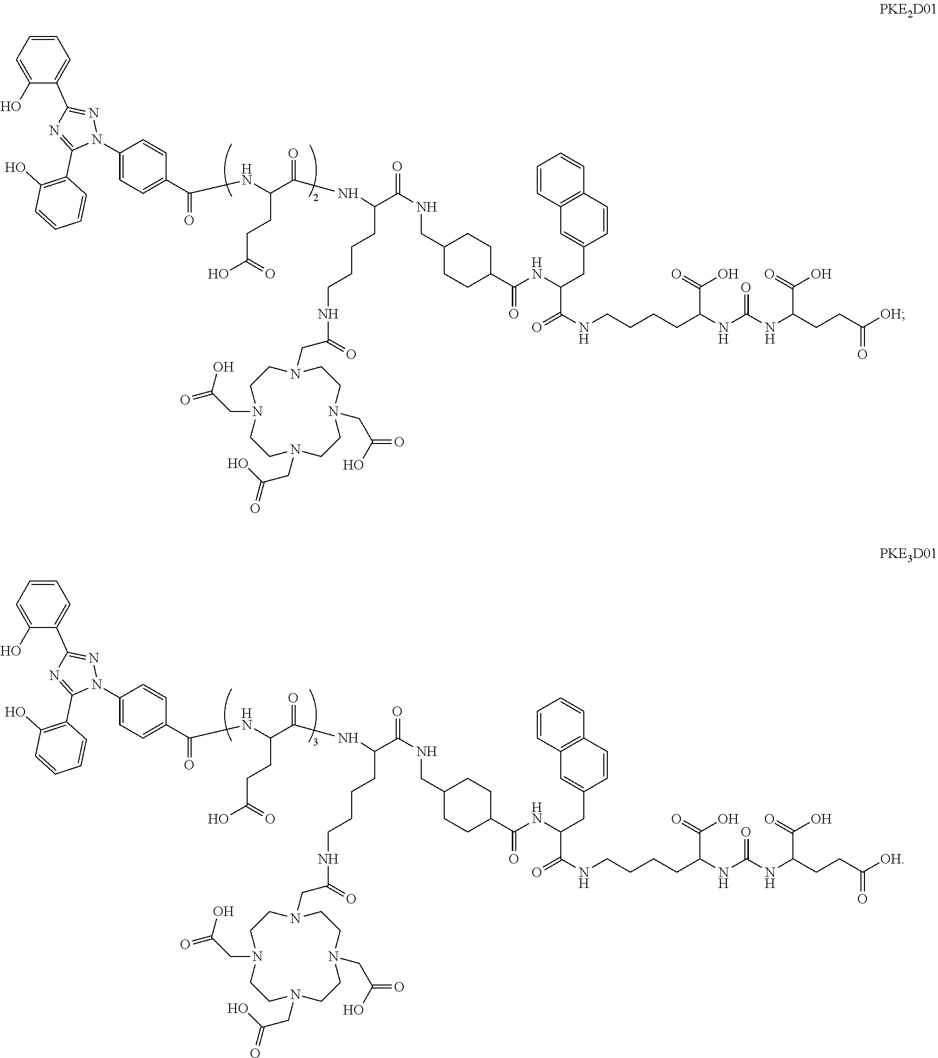

The present disclosure further provides a preparation method of the Glu-urea compound. When X is absent or is linking group 1, the preparation method includes the following steps:
subjecting Polypeptide compound 1 to first substitution with an R₁ active compound to obtain Intermediate 1; and
subjecting Intermediate 1 to deprotection of an R₃ protecting group and then second substitution with an R₂ active compound in sequence to obtain the Glu-urea compound; where
one of the R₁ active compound and the R₂ active compound is selected from the group consisting of

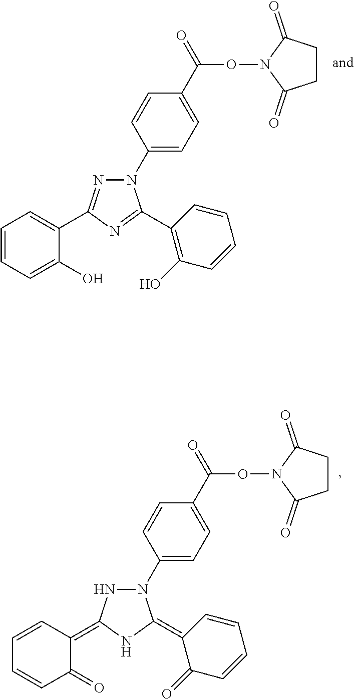 [SMILES: O=C(ON1C(=O)CCC1=O)c1ccc(-n2nc(-c3ccccc3O)nc2-c2ccccc2O)cc1 ; O=C1C=CC=C/C1=C1/N/C(=C2/C=CC=CC2=O)N(c2ccc(C(=O)ON3C(=O)CCC3=O)cc2)N1]

while the other is one selected from the group consisting of:

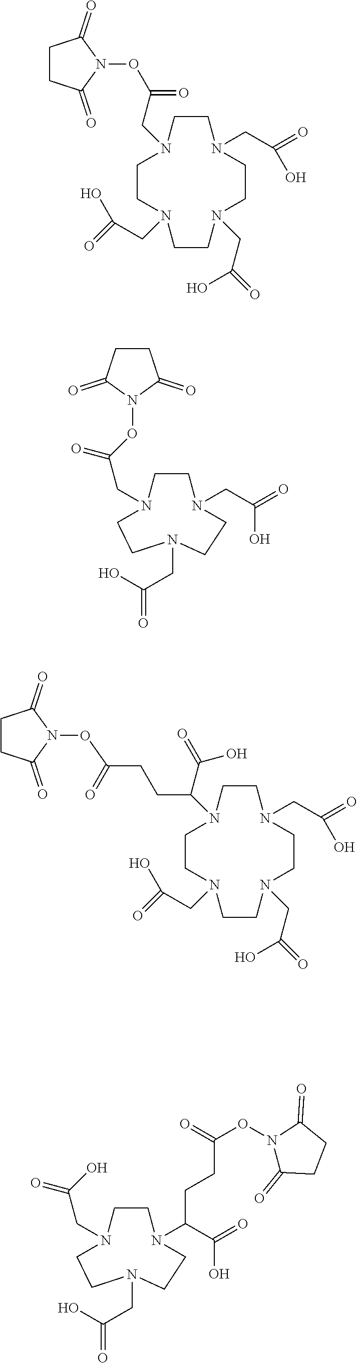 [SMILES: O=C(O)CN1CCN(CC(=O)O)CCN(CC(=O)ON2C(=O)CCC2=O)CCN(CC(=O)O)CC1 ; O=C(O)CN1CCN(CC(=O)O)CCN(CC(=O)ON2C(=O)CCC2=O)CC1 ; O=C(O)CN1CCN(CC(=O)O)CCN(C(CCC(=O)ON2C(=O)CCC2=O)C(=O)O)CCN(CC(=O)O)CC1 ; O=C(O)CN1CCN(CC(=O)O)CCN(C(CCC(=O)ON2C(=O)CCC2=O)C(=O)O)CC1]

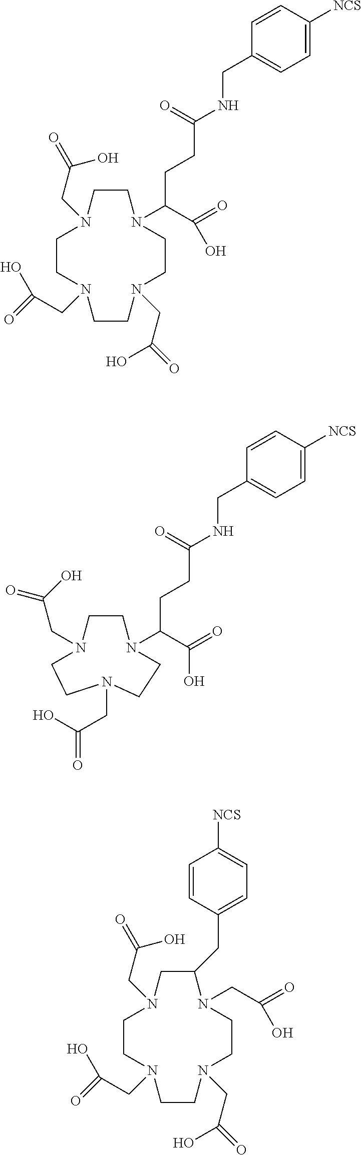

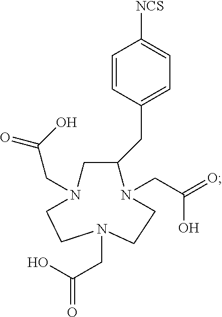

when Polypeptide compound 1 has the following structure:

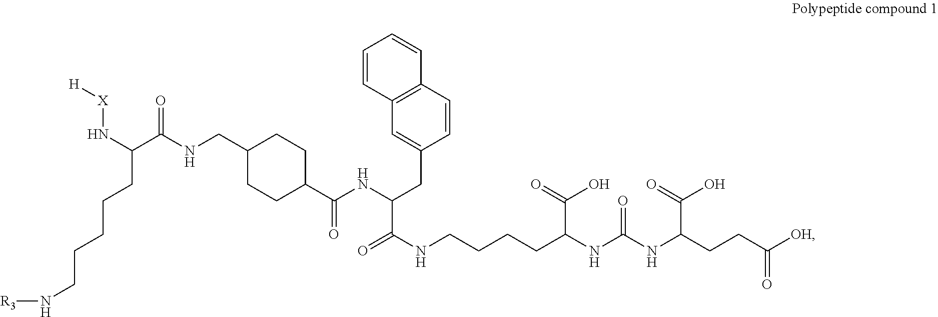

then Intermediate 1 has the following structure:

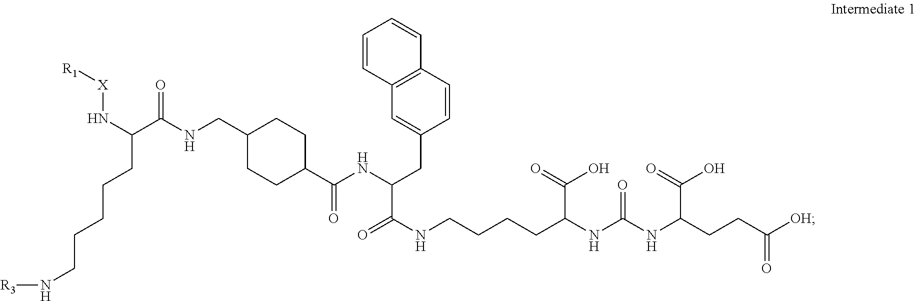

and
when Polypeptide compound 1 has the following structure:

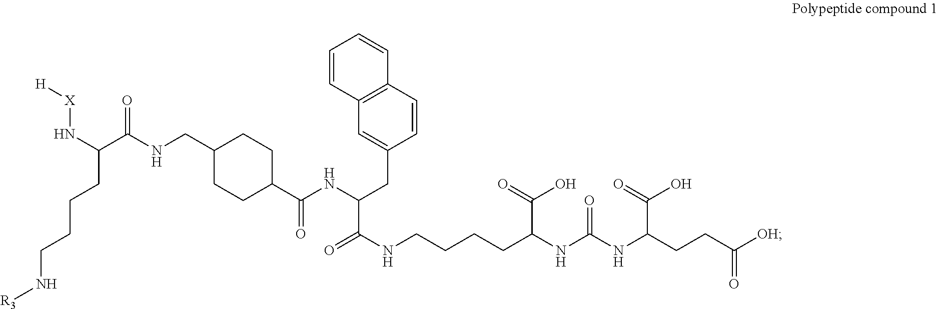

then Intermediate 1 has the following structure:

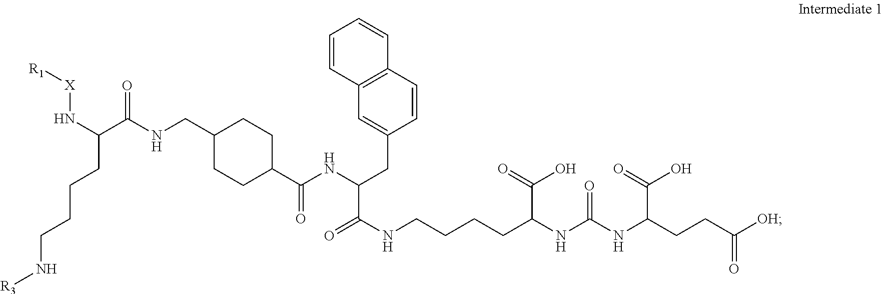

X and R₁ in Polypeptide compound 1 and Intermediate 1 are the same as X and R₁ in formula I-1 or formula 1-2; and R₃ is selected from the group consisting of a Boc protecting group, a DDE protecting group, and an Fmoc protecting group.

In the present disclosure, unless otherwise specified, all materials and equipment used are commercially available items in the art.

In the present disclosure, Polypeptide compound 1 is subjected to first substitution with an R₁ active compound to obtain Intermediate 1.

In the present disclosure, the Polypeptide compound 1 and the R₁ active compound are at a molar ratio of preferably 1:(1-5), more preferably 1:(2-3). The first substitution is preferably conducted in the presence of a high boiling point solvent and an alkaline reagent; the high boiling point solvent preferably includes one or more of NMP, DMSO, and DMF; there is no particular limitation to a dosage of the high boiling point solvent, as long as it can dissolve the Polypeptide compound 1 to ensure a smooth progress of the first substitution. The alkaline reagent is preferably an organic alkali, and the organic alkali preferably includes TEA and/or N,N-diisopropylethylamine (DIPEA); Polypeptide compound 1 and the alkaline reagent are at a molar ratio of preferably 1:(1-10), more preferably 1:(3-5). The first substitution is conducted at preferably 25° C. to 60° C., more preferably 25° C. to 40° C. for preferably 1 h to 24 h, more preferably 5 h to 12 h.

In the present disclosure, workup is preferably conducted after the first substitution is completed; the workup preferably includes: subjecting an obtained first substitution system to reverse-phase high-performance chromatography column purification, and freeze-drying to obtain Intermediate 1. The reverse-phase high-performance chromatography column purification includes the following steps: a chromatographic column is a reverse-phase C18 semi-preparative column; mobile phase A is preferably water+0.1% TFA, and mobile phase B is preferably acetonitrile+0.1% TFA; an elution method is preferably gradient elution; the gradient elution lasts for preferably 0 min to 30 min: a volume fraction of the mobile phase B is increased from 10% to 90%, and a flow rate of the mobile phase is preferably 3 mL/min. There are no special limitations on temperature and time of the freeze-drying, as long as the product is freeze-dried to constant weight (i.e., freeze-drying).

In the present disclosure, Intermediate 1 is subjected to deprotection of R₃ protecting group and then second substitution with an R₂ active compound in sequence to obtain the Glu-urea compound.

In the present disclosure, the deprotection of the R₃ protecting group is preferably conducted in the presence of a hydrazine hydrate solution or TFA; hydrazine hydrate in the hydrazine hydrate solution has a mass fraction of preferably 1% to 10%, more preferably 3% to 5%; there is no special limitation on a dosage of the hydrazine hydrate solution and TFA, as long as the protecting group (DDE, Boc, or Fmoc) can be removed. The deprotection of the R₃ protecting group is conducted at preferably 0° C. to 37° C., more preferably 25° C. for preferably 1 h to 12 h, more preferably 2 h to 5 h.

In the present disclosure, the conditions for the second substitution and the workup after the second substitution are preferably the same as the conditions and the workup for the first substitution, and will not be repeated here.

In the present disclosure, when X is absent or is linking group 1, a preparation route of the Glu-urea compound is as follows:

### Preparation route for formula I-1

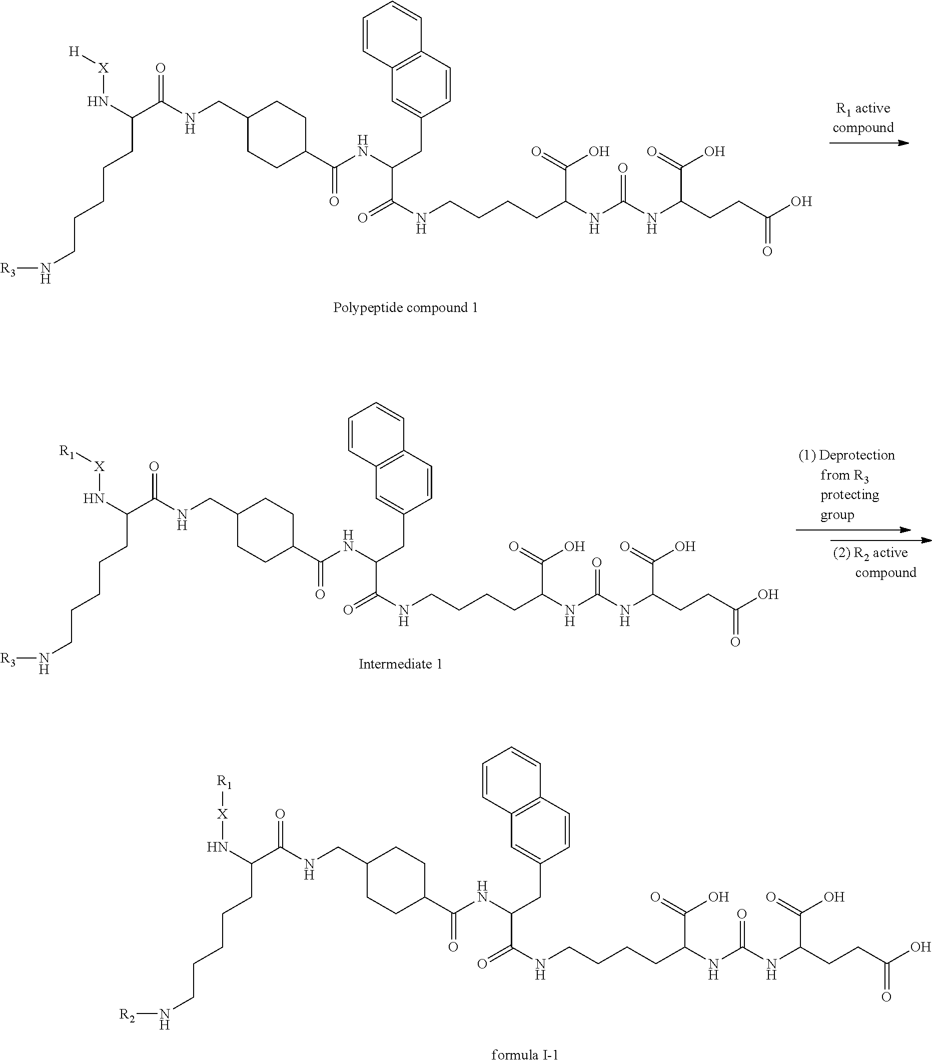

### Preparation route for formula I-2

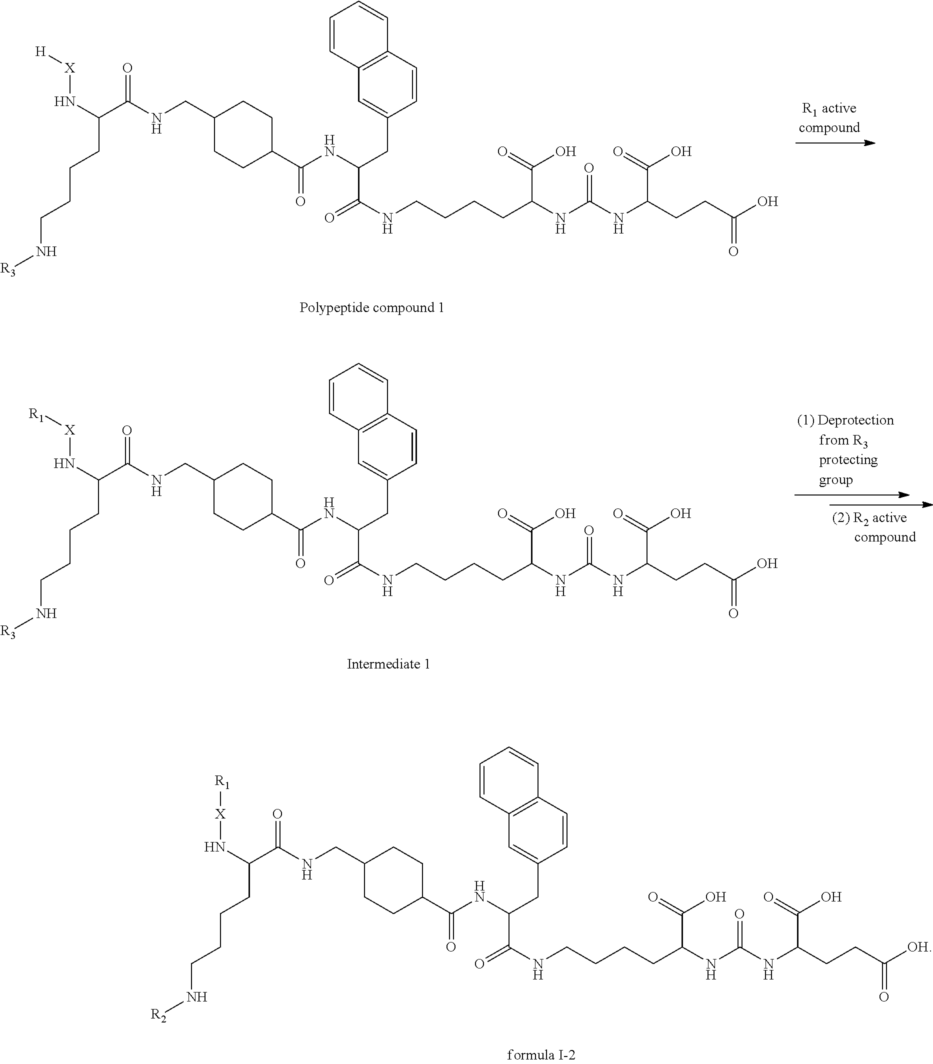

The present disclosure further provides a preparation method of the Glu-urea compound. When X is linking group 2, the preparation method includes the following steps:
subjecting Polypeptide compound 2 to third substitution with R₁-L to obtain intermediate 2; and
subjecting Intermediate 2 to fourth substitution with an R₂ active compound to obtain the Glu-urea compound; where
L in R₁-L is one selected from the group consisting of:

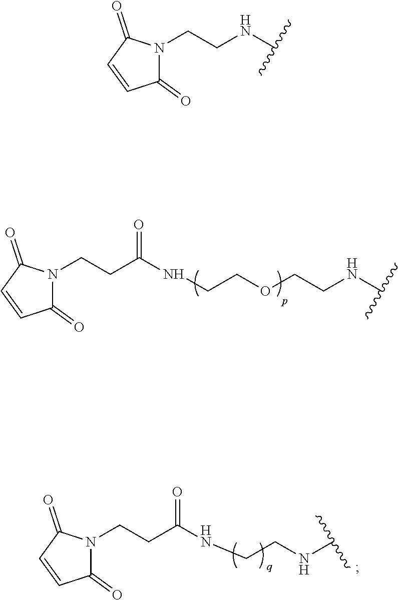

p and q in L are the same as those in linking group 2;
when R₁ in R₁-L is selected from the group consisting of

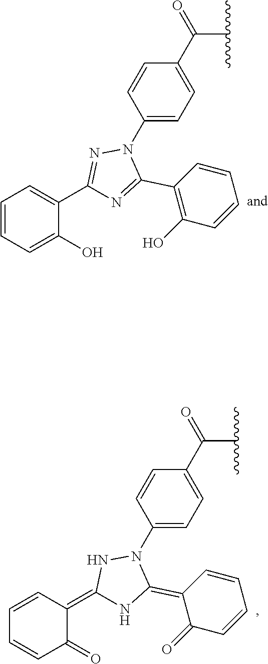 [SMILES: *C(=O)c1ccc(-n2nc(-c3ccccc3O)nc2-c2ccccc2O)cc1 ; *C(=O)c1ccc(N2N/C(=C3\C=CC=CC3=O)N/C2=C2/C=CC=CC2=O)cc1]

the R₂ active compound is one selected from the group consisting of:

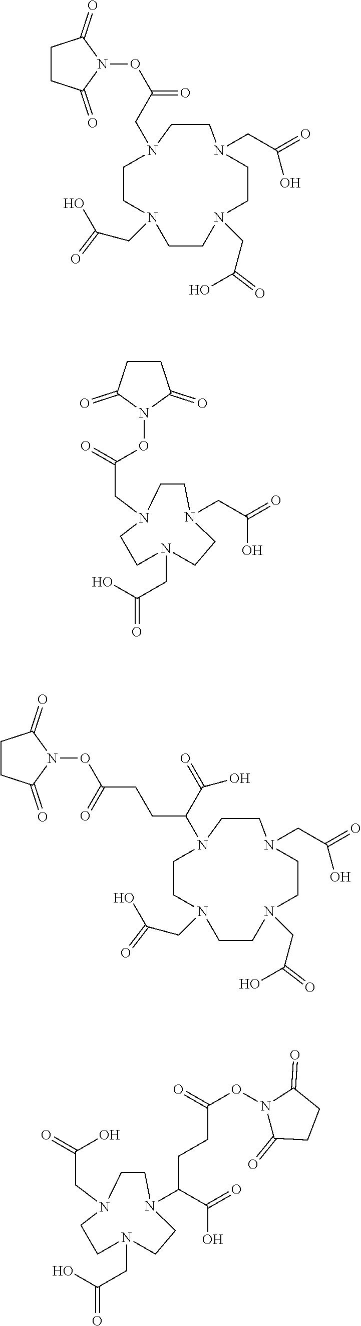 [SMILES: O=C(O)CN1CCN(CC(=O)O)CCN(CC(=O)ON2C(=O)CCC2=O)CCN(CC(=O)O)CC1 ; O=C(O)CN1CCN(CC(=O)O)CCN(CC(=O)ON2C(=O)CCC2=O)CC1 ; O=C(O)CN1CCN(CC(=O)O)CCN(C(CCC(=O)ON2C(=O)CCC2=O)C(=O)O)CCN(CC(=O)O)CC1 ; O=C(O)CN1CCN(CC(=O)O)CCN(C(CCC(=O)ON2C(=O)CCC2=O)C(=O)O)CC1]

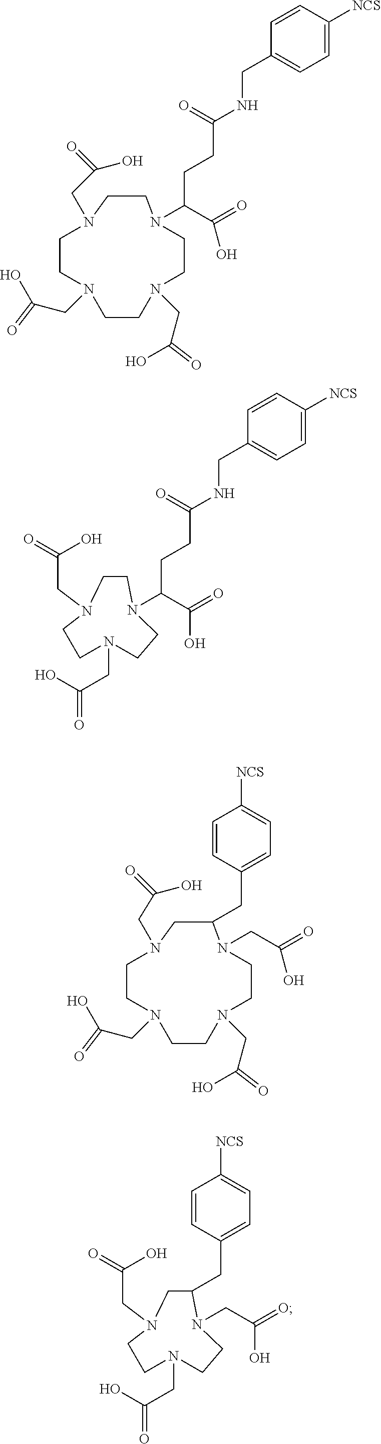

when R₁ in R₁-L is the group to be labeled, the R₂ active compound is selected from the group consisting of

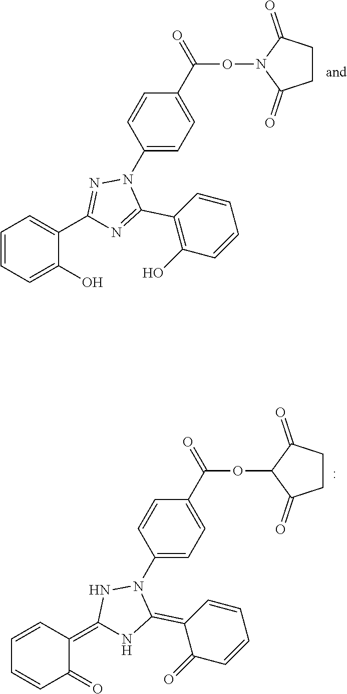 [SMILES: O=C(ON1C(=O)CCC1=O)c1ccc(-n2nc(-c3ccccc3O)nc2-c2ccccc2O)cc1 ; O=C1C=CC=C/C1=C1/N/C(=C2/C=CC=CC2=O)N(c2ccc(C(=O)OC3C(=O)CCC3=O)cc2)N1]

when Polypeptide compound 2 has the following structure:

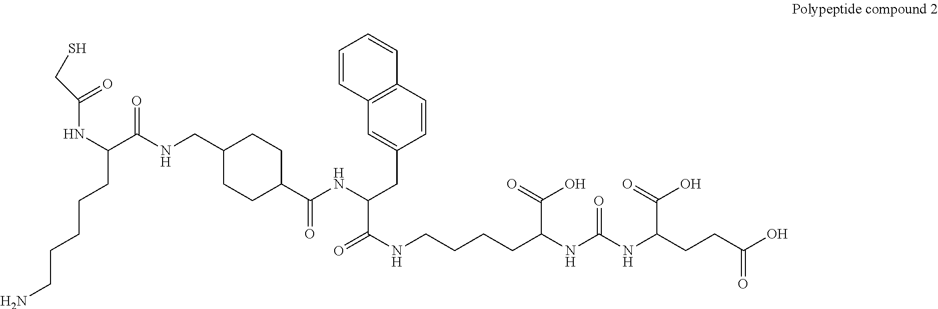 [SMILES: NCCCCCC(NC(=O)CS)C(=O)NCC1CCC(C(=O)NC(Cc2ccc3ccccc3c2)C(=O)NCCCCC(NC(=O)NC(CCC(=O)O)C(=O)O)C(=O)O)CC1]

then Intermediate 2 has the following structure:

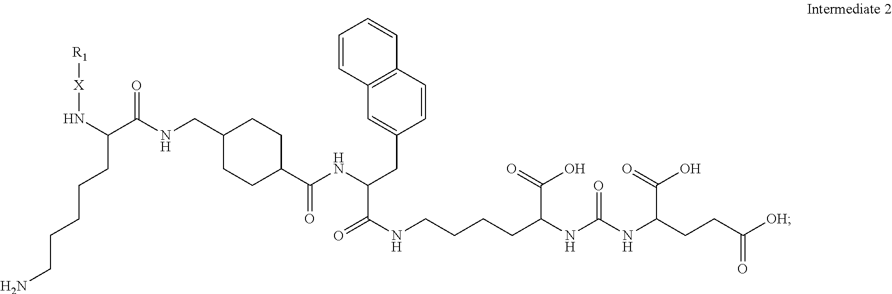

and
when Polypeptide compound 2 has the following structure:

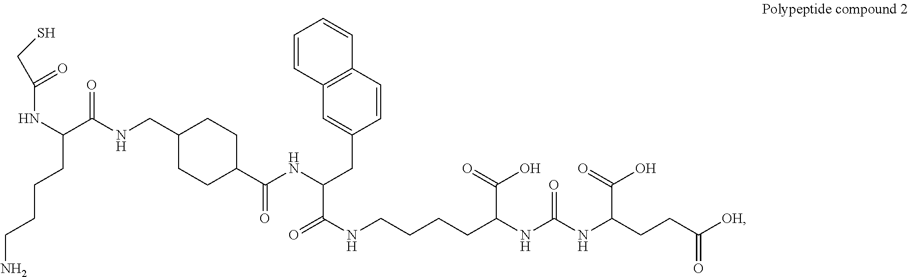 [SMILES: NCCCCC(NC(=O)CS)C(=O)NCC1CCC(C(=O)NC(Cc2ccc3ccccc3c2)C(=O)NCCCCC(NC(=O)NC(CCC(=O)O)C(=O)O)C(=O)O)CC1]

then Intermediate has the following structure:

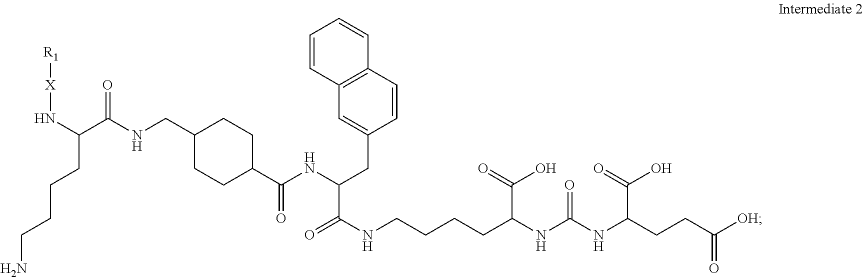

X and R₁ in Polypeptide compound 2 and Intermediate 2 are the same as X and R₁ in formula I-1 or formula I-2.

In the present disclosure, when X is linking group 2, a preparation route of the Glu-urea compound is as follows:

### Preparation route for formula I-1

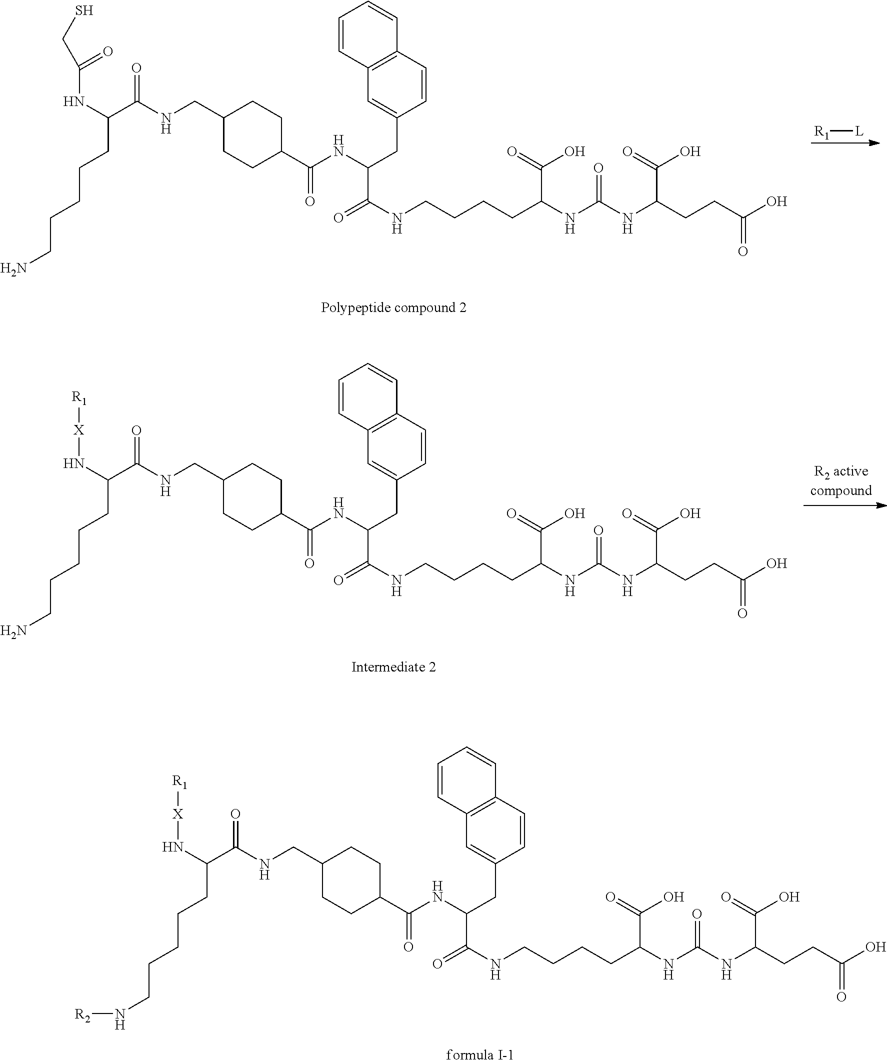

### Preparation route for formula 1-2

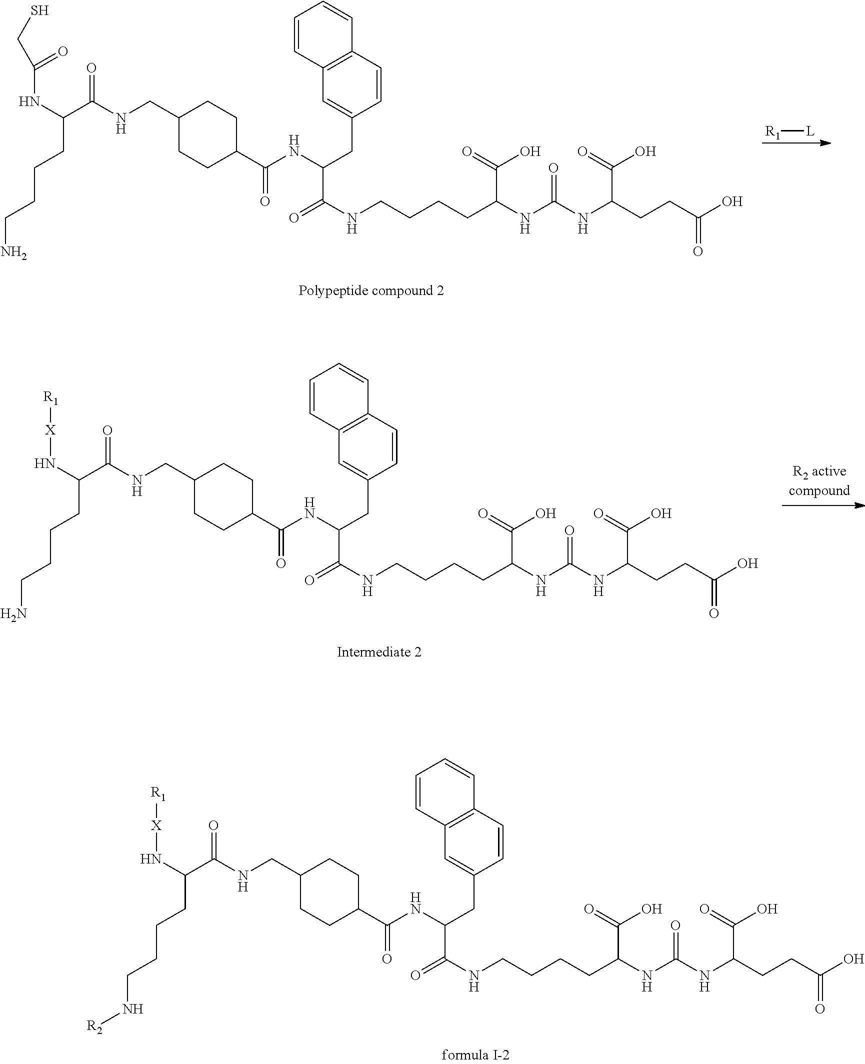

In the present disclosure, Polypeptide compound 2 is subjected to third substitution with R₁-L to obtain Intermediate 2.

In the present disclosure, R₁-L preferably includes DFX-MAL, DOTA-MAL, or DFX-P2-MAL:

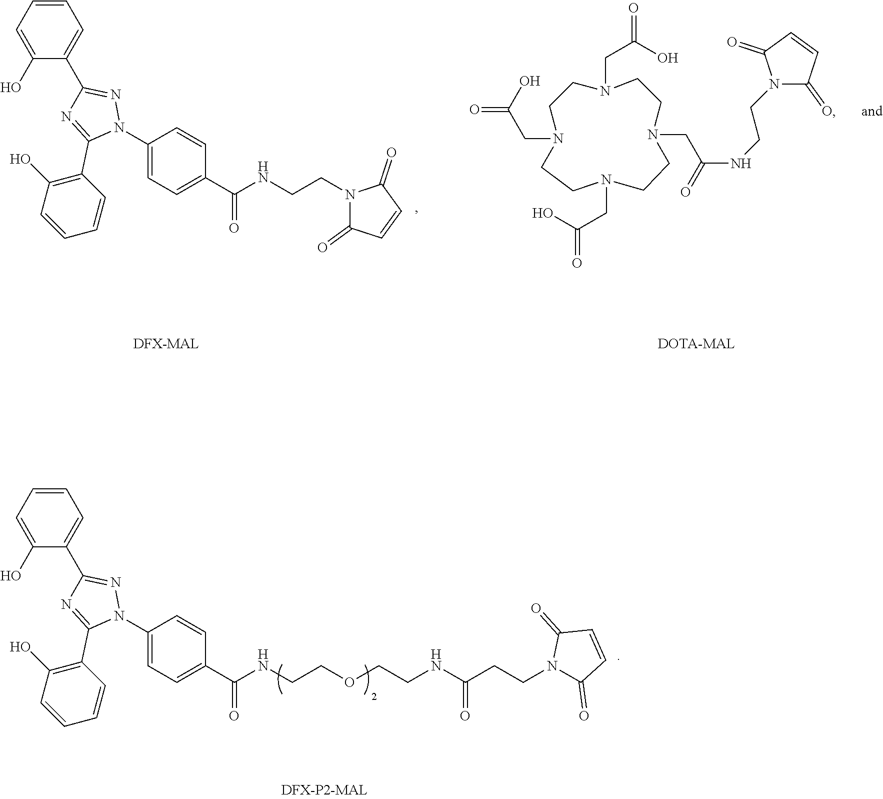

In the present disclosure, Polypeptide compound 2 and a third compound are at a molar ratio of preferably 1:(1-5), more preferably 1:(1.5-2). The third substitution is preferably conducted in the presence of a solvent, and the solvent preferably includes a high boiling point solvent and/or a PBS (phosphate buffer solution), and more preferably is a mixed solvent of the high boiling point solvent and the PBS; the high boiling point solvent preferably includes one or more of NMP, DMSO, and DMF; the PBS has a pH value of preferably 7 to 8, more preferably 7.4 to 7.6; there is no special limitation on the dosage of the solvent, as long as it can dissolve the Polypeptide compound 2 to ensure a smooth progress of the second substitution. The conditions for the third substitution and the workup after the third substitution are preferably the same as the conditions and the post-treatment for the first substitution, and will not be repeated here.

In the present disclosure, Intermediate 2 is subjected to fourth substitution with an R₂ active compound to obtain the Glu-urea compound.

In the present disclosure, the R₂ active compound preferably includes a DOTA or DFX active compound bearing an active reactive group -NHS or -SCN. Intermediate 2 and the R₂ active compound are at a molar ratio of preferably 1:(1-5), more preferably 1:(2-2.5). The conditions for the fourth substitution and the post-treatment after the fourth substitution are preferably the same as the conditions and the post-treatment for the first substitution, and will not be repeated here.

The present disclosure further provides a pharmaceutically acceptable salt of a Glu-urea compound, where the pharmaceutically acceptable salt is prepared by subjecting the Glu-urea compound to a reaction with an acid or an alkali; and the Glu-urea compound is the Glu-urea compound described above or a Glu-urea compound prepared by the preparation method. The pharmaceutically acceptable salt preferably includes a TFA salt, phosphate, formate, acetate, a potassium salt, or a sodium salt. The acid preferably includes TFA, hydrochloric acid, formic acid, or acetic acid; and the alkali preferably includes potassium hydroxide or sodium hydroxide. There are no special restrictions on the conditions of the reaction, and the conditions of the salt-forming reaction well known to those skilled in the art can be used.

The present disclosure further provides a nuclide-targeted probe, where the nuclide-targeted probe is prepared by subjecting the group to be labeled in the Glu-urea compound or the pharmaceutically acceptable salt thereof to complexation with a labeled nuclide. The labeling nuclide is at least one selected from the group consisting of ¹⁸F, ⁴⁷Sc, ⁶⁴Cu, ⁶⁷Cu, ⁶⁷Ga, ⁶⁸Ga, ⁸⁹Zr, ⁸⁶Y, ⁸⁹Sr, ⁹⁰Y, ⁹⁹mTc, ¹⁰⁵Rh, ¹⁰⁹Pd, ¹¹¹In, ¹¹⁹Sb, ¹⁴⁹Tb, ¹⁵³Sm, ¹⁵⁷Gd, ¹⁶¹Tb, ¹⁶⁶Ho, ¹⁷⁷Lu, ¹⁸⁶Re, ¹⁸⁸Re, ²⁰¹Tl, ²⁰³Pb, ²¹²Pb, ²¹²Bi, ²¹³Bi, ²²³Ra, ²²⁷Th, and ²²⁵Ac, more preferably the ¹⁷⁷Lu, ⁶⁸Ga, ⁶⁴Cu, ¹⁸F, ⁹⁰Y, or ²²⁵Ac.

The present disclosure further provides a preparation method of the nuclide-targeted probe, including the following steps:
subjecting the Glu-urea compound to complexation with a labeling nuclide to obtain the nuclide-targeted probe; where the Glu-urea compound is the Glu-urea compound described above or a Glu-urea compound prepared by the preparation method. The nuclide-targeted probe is preferably prepared by a wet labeling method or a freeze-drying labeling method.

In the present disclosure, the wet labeling method of the nuclide-targeted probe preferably includes the following steps: mixing a solution of the Glu-urea compound with a solution of the labeling nuclide to allow complexation, and diluting to obtain an injection of the nuclide-targeted probe.

In the present disclosure, a solvent in the solution of Glu-urea compound preferably includes one or more of a buffer solution, water, and an organic solvent; the buffer solution preferably includes an acetic acid-acetate solution or an aluminum chloride-acetate solution, and a pH value of the buffer solution is preferably 3 to 7, more preferably 4 to 6.5; aluminum chloride in the aluminum chloride-acetate solution has a concentration of preferably 0.2 g/L to 1 g/L, more preferably 0.4 g/L; the acetate in the acetic acid-acetate solution and the aluminum chloride-acetate solution independently include one or more of sodium acetate, potassium acetate, and ammonium acetate; the solution of the Glu-urea compound has a concentration of preferably 0.001 mg/mL to 1,000 mg/mL, more preferably 0.01 mg/mL to 1 mg/mL. The mass of the Glu-urea compound and a radioactive activity of the labeling nuclide in the labeling nuclide solution are at a ratio of preferably (20-400) μg: 1 kBq-1000 GBq, more preferably (20-400) μg: (0.037-74000) MBq, and even more preferably (20-200) μg: (0.037-7400) MBq. There is no special limitation on the labeling nuclide solution, and any labeling nuclide solution well known to those skilled in the art can be used, such as a gadolinium chloride hexahydrate (GdCl₃·6H₂O) solution, a ⁶⁸GaCl₃ hydrochloric acid solution, or a ¹⁷⁷LuCl₃ solution; the ⁶⁸GaCl₃ hydrochloric acid solution is preferably obtained by eluting from a germanium-gallium generator.

In the present disclosure, the complexation is conducted at preferably 25° C. to 100° C., more preferably 80° C. to 100° C. for preferably 10 min to 60 min, more preferably 20 min to 30 min. When the complexation is conducted above a room temperature, a resulting complexation system is preferably cooled to the room temperature after the complexation is completed; there is no particular limitation on the cooling, and any cooling method well known to those skilled in the art can be used, such as natural cooling. After the dilution is completed, a resulting diluted system is preferably subjected to sterile membrane filtration to obtain the injection of the nuclide-targeted probe. The dilution is preferably conducted with physiological saline or water for injection. The injection of the nuclide-targeted probe has a radioactive concentration of preferably 0.037 MBq/mL to 3,700 MBq/mL.

In the present disclosure, when the labeling nuclide solution is the gadolinium chloride hexahydrate solution, a pH value of a mixed solution of the Glu-urea compound and the gadolinium chloride hexahydrate solution is first adjusted to preferably 5.0 to 6.5, more preferably 5.5 to 6.0 with an alkali. The alkali preferably includes a KOH solution; the KOH solution has a concentration of preferably 1 mol/L to 4 mol/L, more preferably 2 mol/L to 3 mol/L.

In the present disclosure, a preparation process of the nuclide-targeted probe by the freeze-drying labeling method preferably includes the following steps: freeze-drying and then sealing a solution of the Glu-urea compound to obtain a freeze-dried kit; dissolving the freeze-dried kit in a solvent, adding the labeling nuclide solution to allow complexation, and diluting to obtain the injection of the nuclide-targeted probe. The freeze-drying is preferably conducted by dividing the solution of the Glu-urea compound into a freeze-drying container and then freeze-drying; there is no special limitation on freeze-drying conditions, and the freeze-drying conditions well known to those skilled in the art can be used. Preferably, an auxiliary material is added to the freeze-dried kit as needed; the auxiliary material preferably includes at least one of an excipient, an antioxidant, and a pH regulator; there are no special limitations on the excipient, antioxidant, and pH regulator, and excipients, antioxidants, and pH regulators well known to those skilled in the art can be used. Other preparation conditions of the nuclide-targeted probe are preferably the same as those of the wet labeling method, and will not be repeated here.

In the present disclosure, when the injection of the nuclide-targeted probe prepared by the wet labeling method and the freeze-drying labeling method has a radiochemical purity of less than 95%, preferably the injection of the nuclide-targeted probe is further purified. The purification is preferably conducted using a Sep-Pak C18 separation cartridge; the Sep-Pak C18 separation cartridge is preferably activated and rinsed with absolute ethanol and water in sequence before use. An eluent used in the purification is preferably water and absolute ethanol in sequence. An eluate of the absolute ethanol is collected, the solvent is removed, and then the eluate is diluted to obtain the injection of a high-purity nuclide-targeted probe. The dilution is preferably conducted with the physiological saline or water for injection. The injection of the high-purity nuclide-targeted probe has a radioactive concentration of preferably 0.037 MBq/mL to 3,700 MBq/mL.

In the present disclosure, the preparation method shows simple and easy labeling, desirable stability of the obtained nuclide-targeted probe, and high tumor uptake, and is suitable for industrial production and clinical promotion.

In addition, the present disclosure further provides a pharmaceutically acceptable salt of a nuclide-targeted probe, where the pharmaceutically acceptable salt of the nuclide-targeted probe is prepared by subjecting the nuclide-targeted probe or a Glu-urea compound to a salt-forming reaction; the nuclide-targeted probe is the nuclide-targeted probe described above or a nuclide-targeted probe prepared by the preparation method; and the Glu-urea compound is the Glu-urea compound described above or a Glu-urea compound prepared by the preparation method. The pharmaceutically acceptable salt preferably includes a TFA salt, phosphate, formate, acetate, a potassium salt, or a sodium salt. There is no particular limitation on a preparation method of the pharmaceutically acceptable salt of the nuclide-targeted probe, and any preparation method of the pharmaceutically acceptable salt well known to those skilled in the art can be used.

Moreover, the present disclosure further provides a pharmaceutical composition, including an active component and a pharmaceutically acceptable auxiliary material; where the active component is one or more selected from the group consisting of a Glu-urea compound, a nuclide-targeted probe, the pharmaceutically acceptable salt of the Glu-urea compound, and the pharmaceutically acceptable salt of the nuclide-targeted probe; the Glu-urea compound is the Glu-urea compound described above or a Glu-urea compound prepared by the preparation method; and the nuclide-targeted probe is the nuclide-targeted probe described above or a nuclide-targeted probe prepared by the preparation method. There are no special limitations on the pharmaceutically acceptable auxiliary material, and pharmaceutically acceptable auxiliary materials well known to those skilled in the art can be used. When the active component includes the nuclide-targeted probe and/or the pharmaceutically acceptable salt of the nuclide-targeted probe, a dosage form of the pharmaceutical composition is preferably an injection, which is preferably administered by intravenous injection. When the active component does not include the nuclide-targeted probe and pharmaceutically acceptable salt thereof, there are no special limitations on the dosage form and administration method of the pharmaceutical composition, and the dosage forms and administration methods well known to those skilled in the art can be used.

Moreover, the present disclosure further provides the use of the Glu-urea compound, a Glu-urea compound prepared by the preparation method, the pharmaceutically acceptable salt of the Glu-urea compound, the nuclide-targeted probe, a nuclide-targeted probe prepared by the preparation method, the pharmaceutically acceptable salt of the nuclide-targeted probe, or the pharmaceutical composition in preparation of a therapeutic drug or a diagnostic drug for a prostate-specific membrane antigen (PSMA) protein-mediated disease. In the present disclosure, the PSMA protein-mediated disease preferably includes a tumor; and the tumor is preferably one or more selected from the group consisting of prostate cancer, breast cancer, ovarian cancer, liver cancer, lung cancer, colorectal cancer, skeletal tissue sarcoma, connective tissue sarcoma, renal cell carcinoma, gastric cancer, pancreatic cancer, nasopharyngeal cancer, head and neck cancer, neuroendocrine tumor, and cutaneous melanoma. In the present disclosure, the diagnosis includes SPECT, PET, and MRI; and the therapy includes a nuclide-targeted therapy and/or chemotherapy.

In order to further illustrate the present disclosure, the Glu-urea compound and the preparation method and the use thereof, the nuclide-targeted probe and the preparation method and the use thereof, and the pharmaceutical composition provided by the present disclosure are described in detail below with reference to the accompanying drawings and examples, but the accompanying drawings and the examples should not be construed as limiting the protection scope of the present disclosure.

### EXAMPLE 1

### Synthesis of PKND01

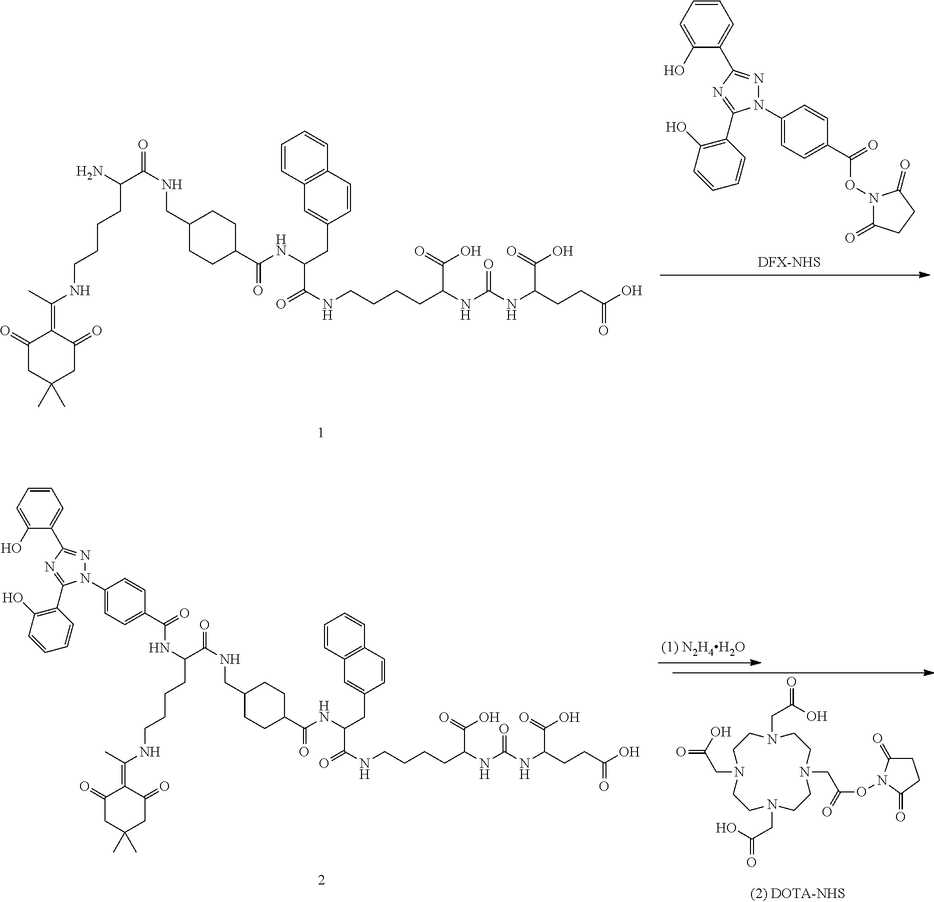 [SMILES: CC(NCCCCC(N)C(=O)NCC1CCC(C(=O)NC(Cc2ccc3ccccc3c2)C(=O)NCCCCC(NC(=O)NC(CCC(=O)O)C(=O)O)C(=O)O)CC1)=C1C(=O)CC(C)(C)CC1=O ; O=C(ON1C(=O)CCC1=O)c1ccc(-n2nc(-c3ccccc3O)nc2-c2ccccc2O)cc1 ; CC(NCCCCC(NC(=O)c1ccc(-n2nc(-c3ccccc3O)nc2-c2ccccc2O)cc1)C(=O)NCC1CCC(C(=O)NC(Cc2ccc3ccccc3c2)C(=O)NCCCCC(NC(=O)NC(CCC(=O)O)C(=O)O)C(=O)O)CC1)=C1C(=O)CC(C)(C)CC1=O ; O=C(O)CN1CCN(CC(=O)O)CCN(CC(=O)ON2C(=O)CCC2=O)CCN(CC(=O)O)CC1]

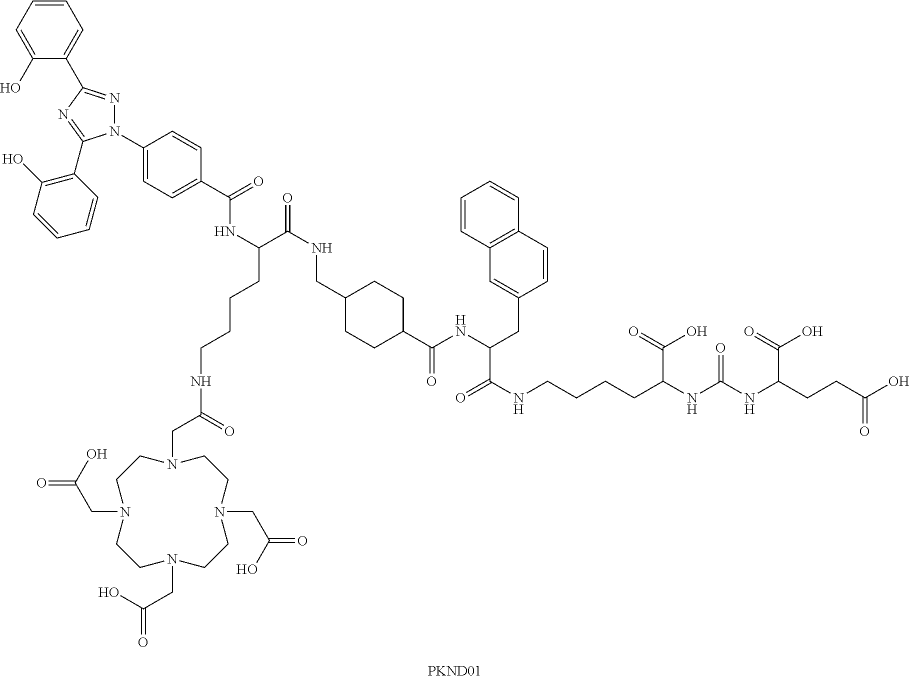 [SMILES: O=C(O)CCC(NC(=O)NC(CCCCNC(=O)C(Cc1ccc2ccccc2c1)NC(=O)C1CCC(CNC(=O)C(CCCCNC(=O)CN2CCN(CC(=O)O)CCN(CC(=O)O)CCN(CC(=O)O)CC2)NC(=O)c2ccc(-n3nc(-c4ccccc4O)nc3-c3ccccc3O)cc2)CC1)C(=O)O)C(=O)O ; "PKND01"]

(1) Synthesis of compound 2: compound 1 (3.16 μmol) was added into a 1.5 mL centrifuge tube, dissolved in DMSO (0.5 mL), raw materials DFX-NHS (9.49 μmol) and DIPEA (15.82 μmol) were added, reacted for 12 h at 25° C., purified by HPLC and freeze-dried (at −65° C.) to obtain a white solid, namely compound 2 (4 mg, yield 97%, purity 98%). HPLC purification included: reverse-phase C18 semi-preparative column (10 mm×250 mm); mobile phase A: water+0.1% TFA; mobile phase B: acetonitrile+0.1% TFA; gradient elution included: 0 min to 30 min: a volume fraction of the mobile phase B increased from 10% to 90%, and a flow rate of the mobile phase was 3 mL/min. Compound 2: ESI MS [M+H]⁺ for C₇₀H₈₃N₁₀O₁₅, calculated 1303.60, found 1303.25.

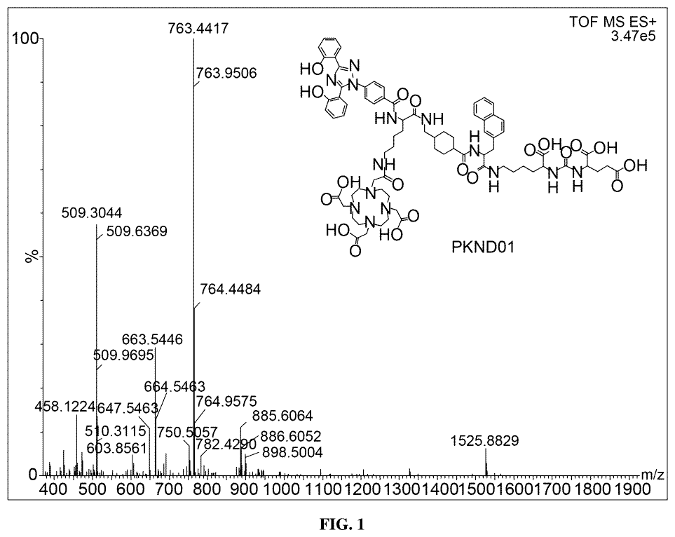

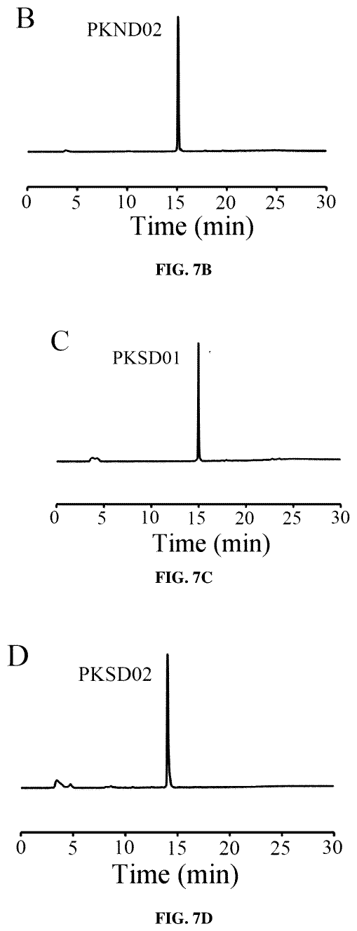

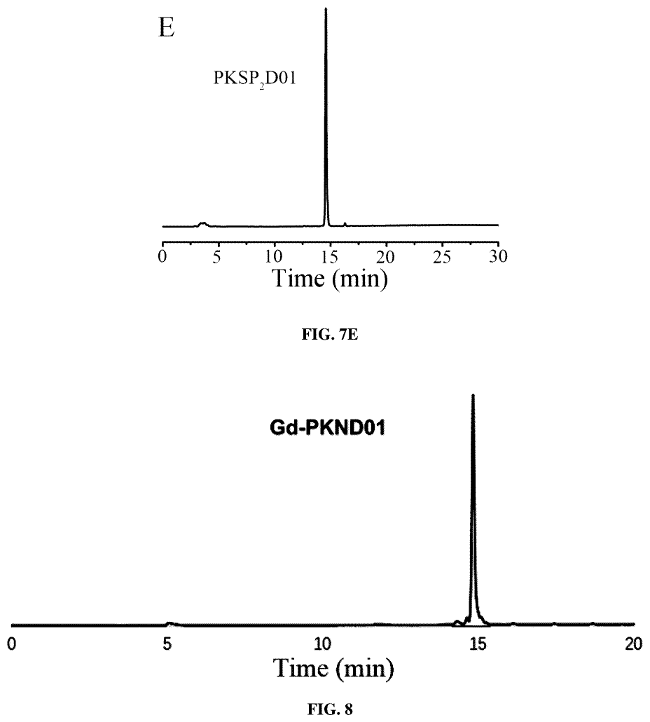

(2) Synthesis of PKND01: compound 2 (3.06 μmol) was added into a 1.5 mL centrifuge tube, hydrazine hydrate (0.5 mL, mass fraction 3%) was added, reacted at 25° C. for 2 h, DMSO (0.5 mL), DOTA-NHS (5.48 μmol), and DIPEA (10.98 μmol) were added, stirred at 25° C. for 12 h, purified by HPLC and freeze-dried (at −65° C.) to obtain compound PKND01 (1.6 mg, yield 47.7%) with a purity of greater than 95% after identification. HPLC purification included: reverse-phase C18 semi-preparative column (10 mm×250 mm); mobile phase A: water+0.1% TFA; mobile phase B: acetonitrile+0.1% TFA; gradient elution included: 0 min to 30 min: a volume fraction of the mobile phase B increased from 10% to 90%, and a flow rate of the mobile phase was 3 mL/min. PKND01: ESI MS [M+H]⁺ for C₇₆H₉₇N₁₄O₂₀, calculated 1525.69, found 1525.88, as shown in FIG. 1; the HPLC purity analysis is shown in FIG. 7A, and the purity was greater than 95%.

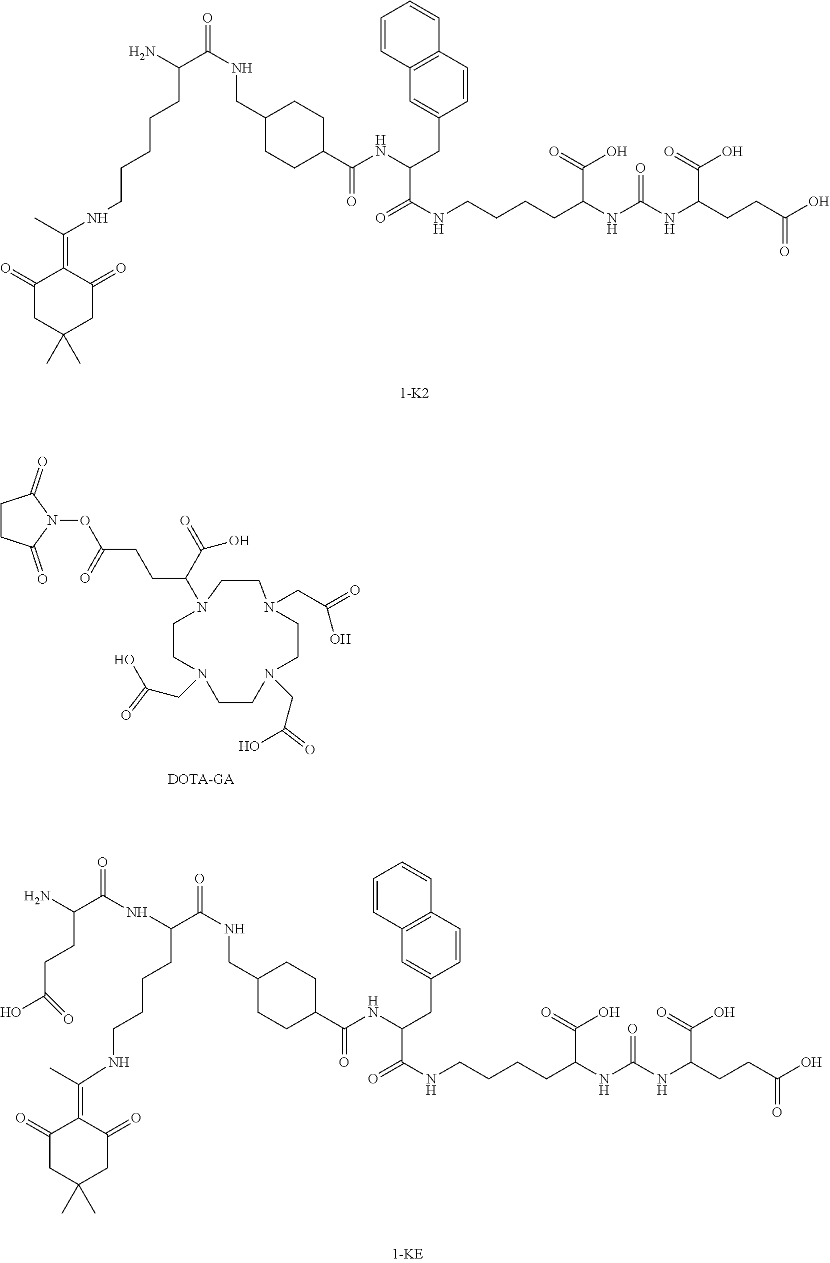 [SMILES: CC(NCCCCCC(N)C(=O)NCC1CCC(C(=O)NC(Cc2ccc3ccccc3c2)C(=O)NCCCCC(NC(=O)NC(CCC(=O)O)C(=O)O)C(=O)O)CC1)=C1C(=O)CC(C)(C)CC1=O ; O=C(O)CN1CCN(CC(=O)O)CCN(C(CCC(=O)ON2C(=O)CCC2=O)C(=O)O)CCN(CC(=O)O)CC1 ; CC(NCCCCC(NC(=O)C(N)CCC(=O)O)C(=O)NCC1CCC(C(=O)NC(Cc2ccc3ccccc3c2)C(=O)NCCCCC(NC(=O)NC(CCC(=O)O)C(=O)O)C(=O)O)CC1)=C1C(=O)CC(C)(C)CC1=O]

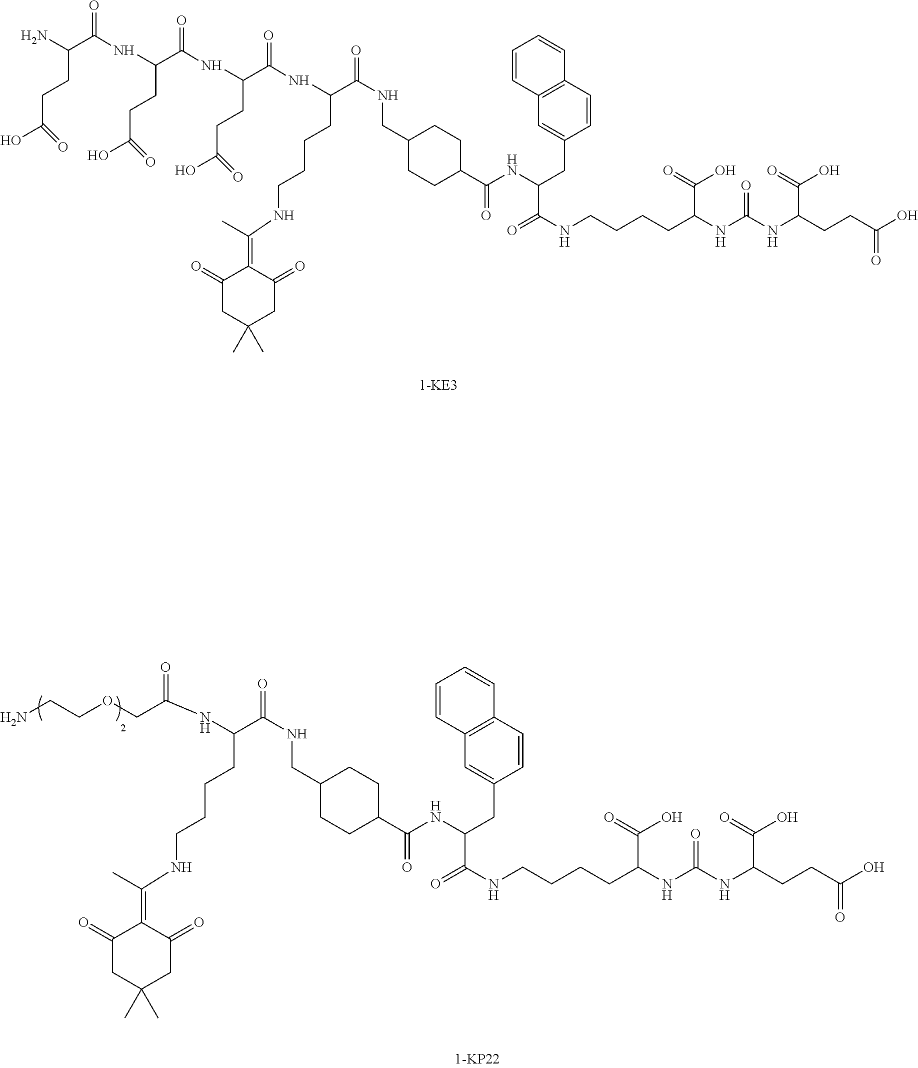

The product PK2ND01 was prepared using the preparation method for PKND01, with the DDE-protected lysine fragment in compound 1 replaced with a 2,7-diaminoheptanoic acid fragment (compound 1-K2).

The product PK2NGD01 was prepared using the preparation method for PKND01, with the DDE-protected lysine fragment in compound 1 replaced with a 2,7-diaminoheptanoic acid fragment (compound 1-K2) and DOTA-NHS replaced with DOTA-GA.

The product PKED01 was prepared using the preparation method for PKND01, with the DDE-protected lysine fragment in compound 1 replaced with compound 1-KE.

The product PKE₃D01 was prepared using the preparation method for PKND01, with the DDE-protected lysine fragment in compound 1 replaced with compound 1-KE3.

The product PKP₂₂D01 was prepared using the preparation method for PKND01, with the DDE-protected lysine fragment in compound 1 replaced with compound 1-KP22.

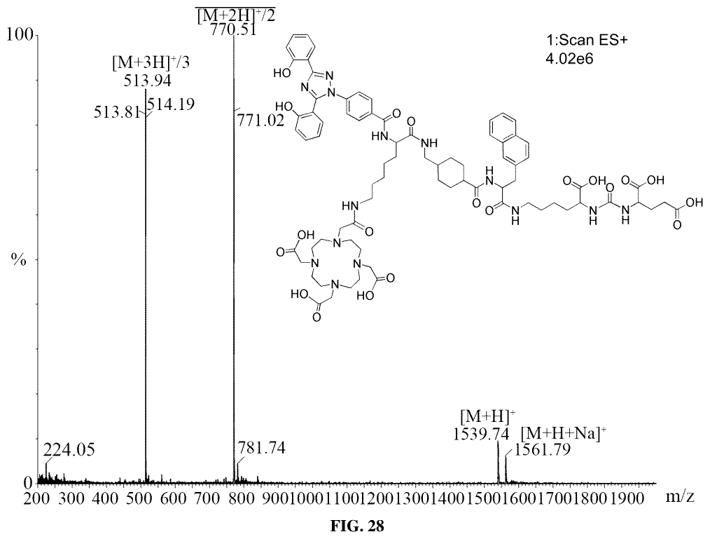

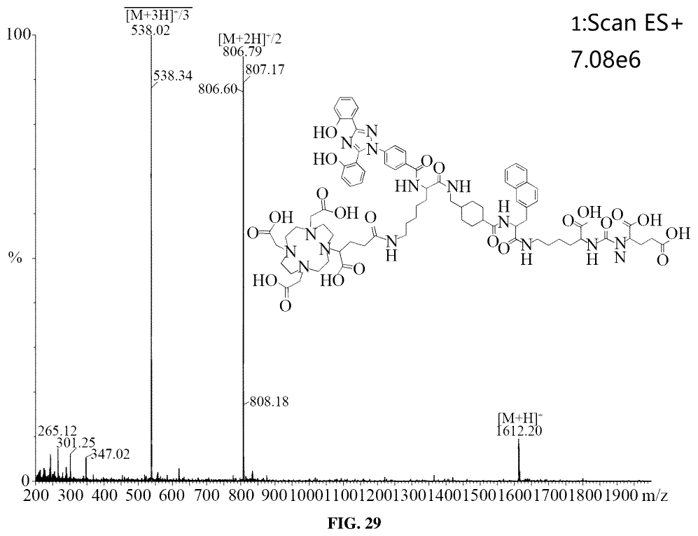

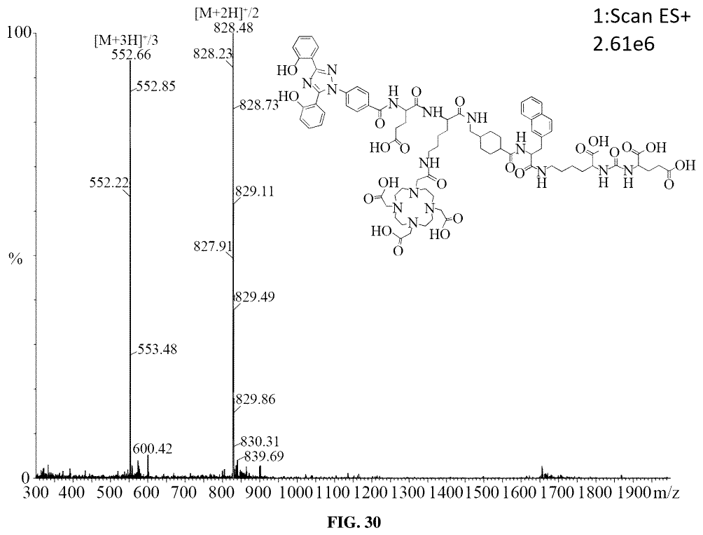

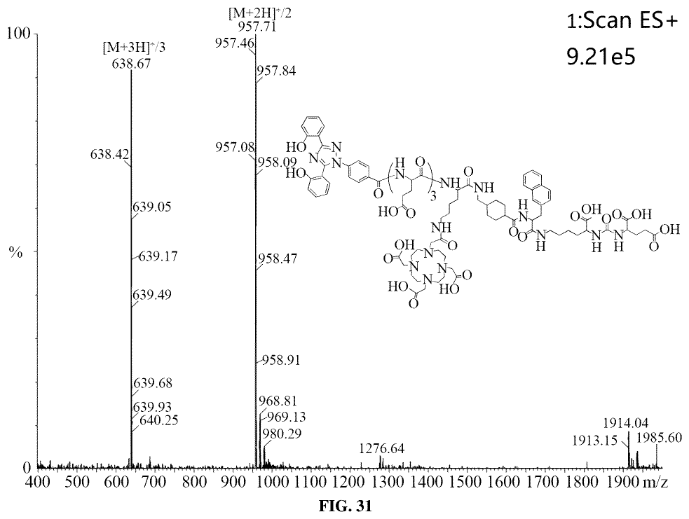

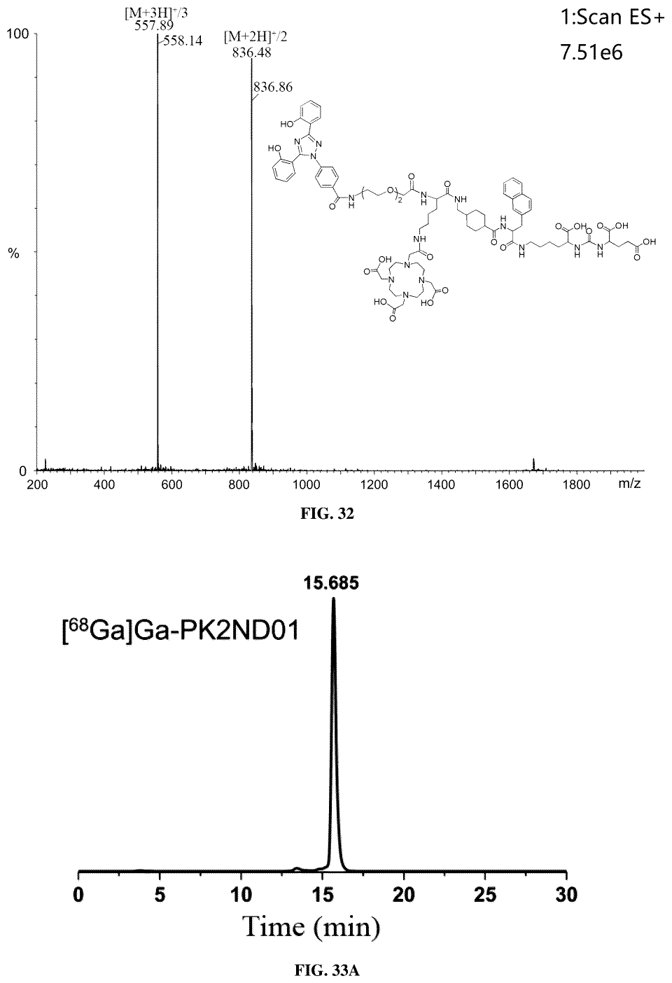

The mass spectra for identification of compound PK2ND01, PK2NGD01, PKED01, PKE₃D01, and PKP₂₂D01 are shown in FIG. 28, FIG. 29, FIG. 30, FIG. 31, and FIG. 32, respectively.

### EXAMPLE 2

### Synthesis of PKND02

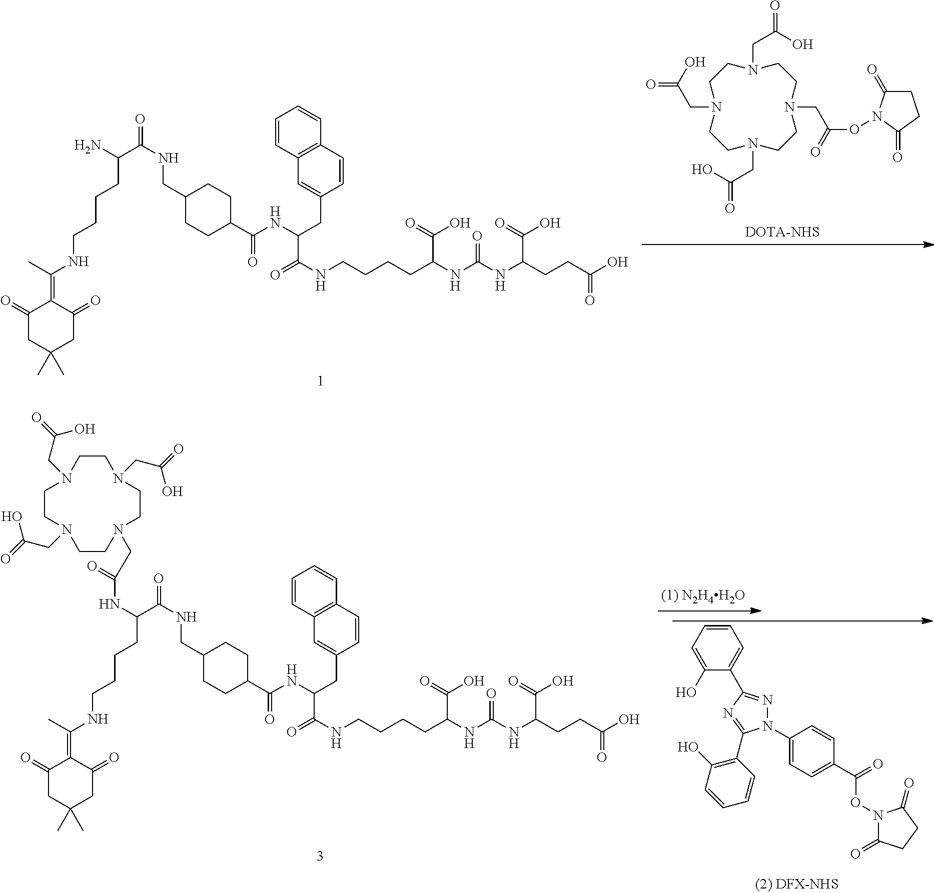 [SMILES: CC(NCCCCC(N)C(=O)NCC1CCC(C(=O)NC(Cc2ccc3ccccc3c2)C(=O)NCCCCC(NC(=O)NC(CCC(=O)O)C(=O)O)C(=O)O)CC1)=C1C(=O)CC(C)(C)CC1=O ; O=C(O)CN1CCN(CC(=O)O)CCN(CC(=O)ON2C(=O)CCC2=O)CCN(CC(=O)O)CC1 ; CC(NCCCCC(NC(=O)CN1CCN(CC(=O)O)CCN(CC(=O)O)CCN(CC(=O)O)CC1)C(=O)NCC1CCC(C(=O)NC(Cc2ccc3ccccc3c2)C(=O)NCCCCC(NC(=O)NC(CCC(=O)O)C(=O)O)C(=O)O)CC1)=C1C(=O)CC(C)(C)CC1=O ; O=C(ON1C(=O)CCC1=O)c1ccc(-n2nc(-c3ccccc3O)nc2-c2ccccc2O)cc1]

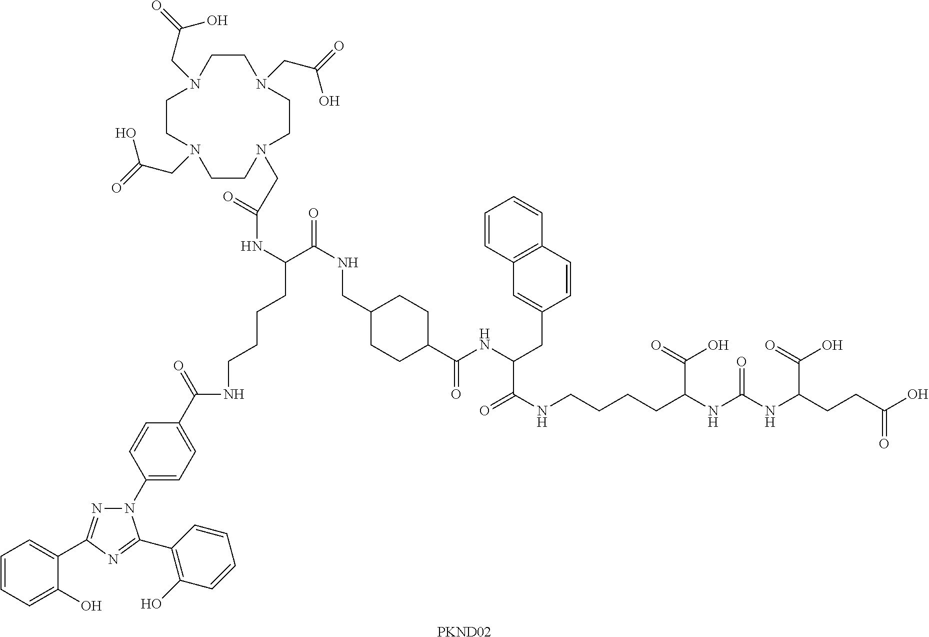 [SMILES: O=C(O)CCC(NC(=O)NC(CCCCNC(=O)C(Cc1ccc2ccccc2c1)NC(=O)C1CCC(CNC(=O)C(CCCCNC(=O)c2ccc(-n3nc(-c4ccccc4O)nc3-c3ccccc3O)cc2)NC(=O)CN2CCN(CC(=O)O)CCN(CC(=O)O)CCN(CC(=O)O)CC2)CC1)C(=O)O)C(=O)O ; "PKND02"]

(1) Synthesis of compound 3: compound 1 (3.16 μmol) was added into a 1.5 mL centrifuge tube, dissolved in DMSO (0.5 mL), and raw materials DOTA-NHS (7.91 μmol) and DIPEA (15.82 μmol) were added, reacted for 12 h at 25° C., purified by HPLC and freeze-dried (at −65° C.) to obtain a white solid, namely compound 3 (3.7 mg, yield 88%, purity 98%). HPLC purification included: reverse-phase C18 semi-preparative column (10 mm×250 mm); mobile phase A: water+0.1% TFA; mobile phase B: acetonitrile+0.1% TFA; gradient elution included: 0 min to 30 min: a volume fraction of the mobile phase B increased from 10% to 90%, and a flow rate of the mobile phase was 3 mL/min. Compound 3: ESI MS [M+H]⁺ for C₆₅H₉₆N₁₁O₁₉, calculated 1335.53, found 1335.24.

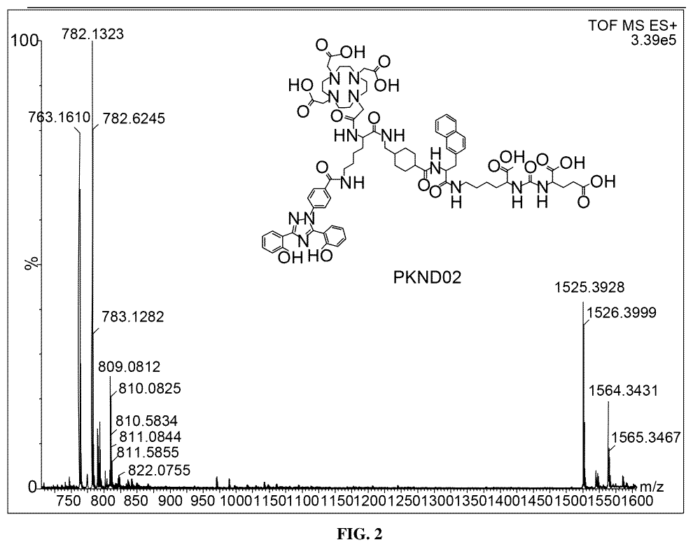

(2) Synthesis of PKND02: compound 3 (3.06 μmol) was added into a 1.5 mL centrifuge tube, hydrazine hydrate (0.5 mL, mass fraction 3%) was added, reacted at 25° C. for 2 h, DMSO (0.5 mL), DOTA-NHS (5.34 μmol) and DIPEA (6.41 μmol) were added, stirred at 25° C. for 12 h, purified by HPLC and freeze-dried (at −65° C.) to obtain compound PKND02 (2 mg, yield 61.53%) with a purity of greater than 95% after identification. HPLC purification included: reverse-phase C18 semi-preparative column (10 mm×250 mm); mobile phase B: acetonitrile+0.1% TFA; gradient elution included: 0 min to 30 min: a volume fraction of the mobile phase B increased from 10% to 90%, and a flow rate of the mobile phase was 3 mL/min. PKND02: ESI MS [M+H]⁺ for C₇₆H₉₇N₁₄O₂₀, calculated 1525.69, found 1525.39, as shown in FIG. 2; the HPLC purity analysis is shown in FIG. 7B, and the purity was greater than 95%.

### EXAMPLE 3

### Synthesis of PKSD01

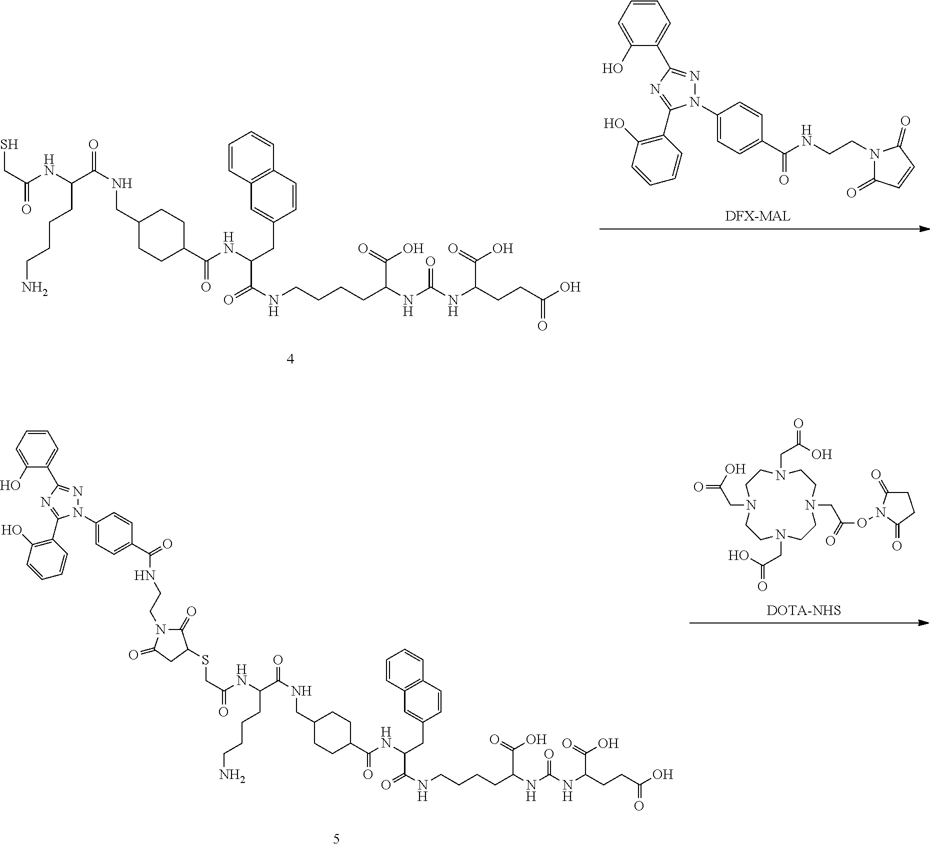 [SMILES: NCCCCC(NC(=O)CS)C(=O)NCC1CCC(C(=O)NC(Cc2ccc3ccccc3c2)C(=O)NCCCCC(NC(=O)NC(CCC(=O)O)C(=O)O)C(=O)O)CC1 ; O=C(NCCN1C(=O)C=CC1=O)c1ccc(-n2nc(-c3ccccc3O)nc2-c2ccccc2O)cc1 ; NCCCCC(NC(=O)CSC1CC(=O)N(CCNC(=O)c2ccc(-n3nc(-c4ccccc4O)nc3-c3ccccc3O)cc2)C1=O)C(=O)NCC1CCC(C(=O)NC(Cc2ccc3ccccc3c2)C(=O)NCCCCC(NC(=O)NC(CCC(=O)O)C(=O)O)C(=O)O)CC1 ; O=C(O)CN1CCN(CC(=O)O)CCN(CC(=O)ON2C(=O)CCC2=O)CCN(CC(=O)O)CC1]

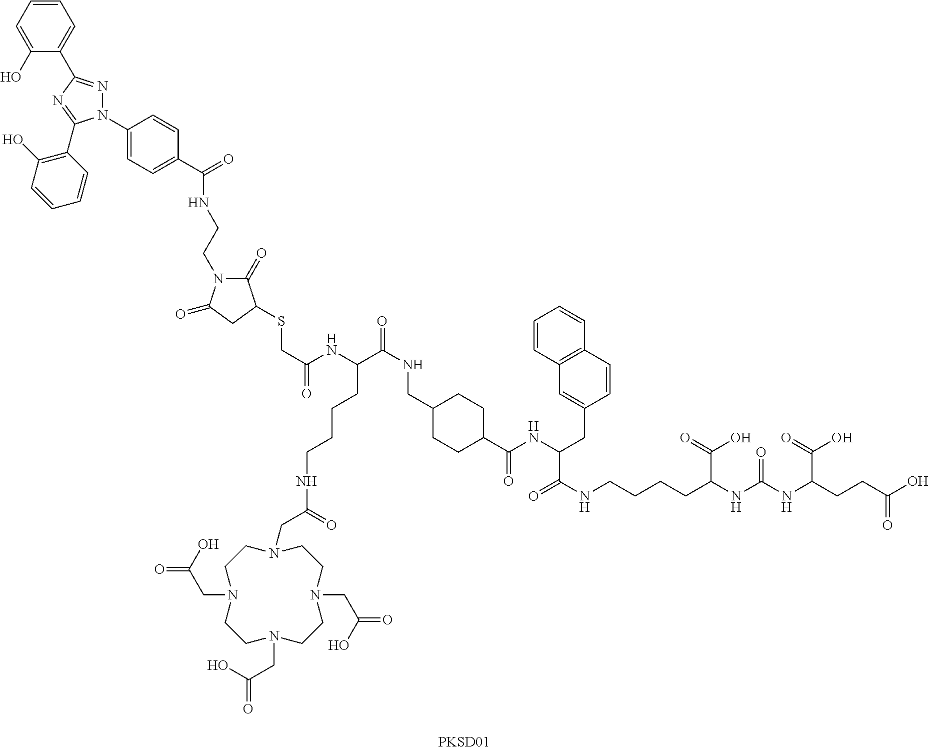 [SMILES: O=C(O)CCC(NC(=O)NC(CCCCNC(=O)C(Cc1ccc2ccccc2c1)NC(=O)C1CCC(CNC(=O)C(CCCCNC(=O)CN2CCN(CC(=O)O)CCN(CC(=O)O)CCN(CC(=O)O)CC2)NC(=O)CSC2CC(=O)N(CCNC(=O)c3ccc(-n4nc(-c5ccccc5O)nc4-c4ccccc4O)cc3)C2=O)CC1)C(=O)O)C(=O)O ; "PKSD01"]

(1) Synthesis of compound 5: a compound 4 (3.03 μmol) was added into a 1.5 mL centrifuge tube, dissolved in DMSO (0.5 mL), raw materials DFX-MAL (6.05 μmol) and PBS solution (0.3 mL) were added, reacted for 12 h at 25° C., purified by HPLC and freeze-dried (at −65° C.) to obtain a white solid, namely compound 5 (2 mg, yield 48.78%, purity 98%). HPLC purification included: reverse-phase C18 semi-preparative column (10 mm×250 mm); mobile phase B: acetonitrile+0.1% TFA; gradient elution included: 0 min to 30 min: a volume fraction of the mobile phase B increased from 10% to 90%, and a flow rate of the mobile phase was 3 mL/min. ESI MS [M+H]⁺ for C₆₈H₈₁N₁₂O₁₆S, calculated 1354.52, found 1354.34.

(2) Synthesis of PKSD01: compound 5 (2 mg, 1 eq., 1.48 μmol) was added into a 1.5 mL centrifuge tube, DMSO (0.5 mL), DOTA-NHS (1.85 mg, 2.5 eq., 3.69 μmol), and DIPEA (0.7 mg, 4 eq., 5.91 μmol) were added, stirred at 25° C. for 12 h, purified by HPLC and freeze-dried (at −65° C.) to obtain compound PKSD01 (1 mg, yield 38.91%) with a purity of greater than 95% after identification. HPLC purification included: reverse-phase C18 semi-preparative column (10 mm×250 mm); mobile phase A: water+0.1% TFA; mobile phase B: acetonitrile+0.1% TFA; gradient elution included: 0min to 30 min: a volume fraction of the mobile phase B increased from 10% to 90%, and a flow rate of the mobile phase was 3 mL/min. PKSD01: ESI MS [M+H]⁺ for C₇₆H₉₇N₁₄O₂₀, calculated 1739.73, found 1739.70, as shown in FIG. 3; the HPLC purity analysis is shown in FIG. 7C, and the purity was greater than 95%.

### EXAMPLE 4

### Synthesis of PKSD02

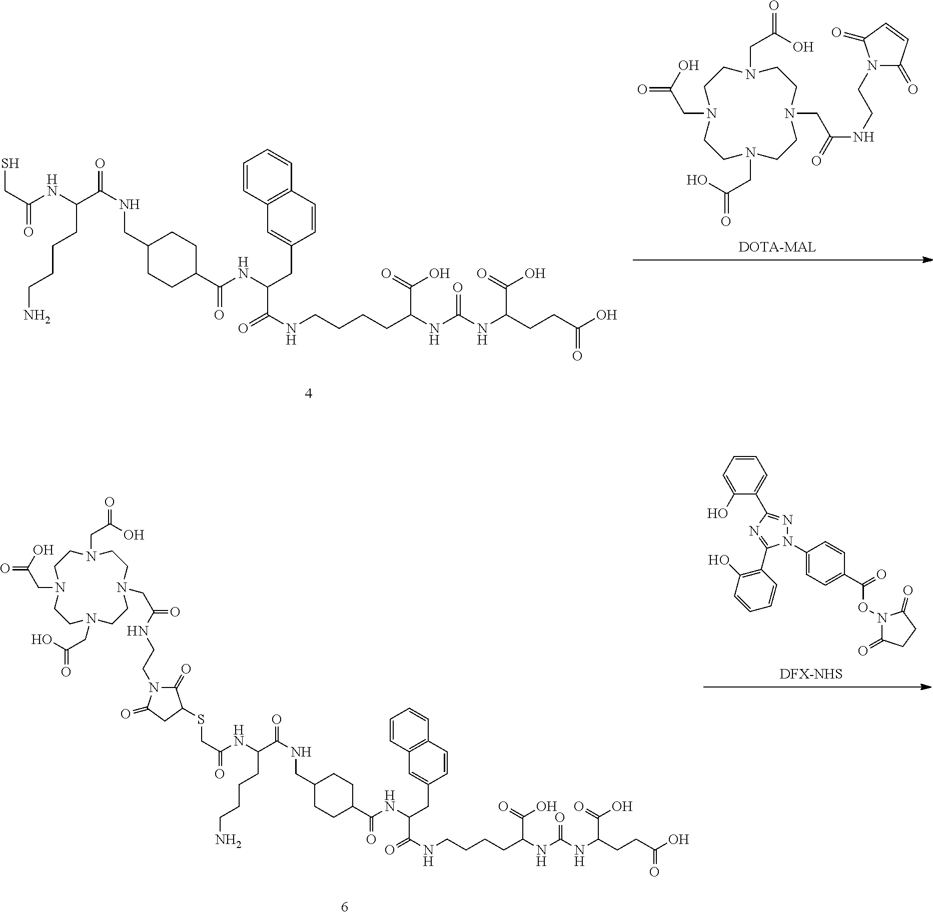 [SMILES: NCCCCC(NC(=O)CS)C(=O)NCC1CCC(C(=O)NC(Cc2ccc3ccccc3c2)C(=O)NCCCCC(NC(=O)NC(CCC(=O)O)C(=O)O)C(=O)O)CC1 ; O=C(O)CN1CCN(CC(=O)O)CCN(CC(=O)NCCN2C(=O)C=CC2=O)CCN(CC(=O)O)CC1 ; NCCCCC(NC(=O)CSC1CC(=O)N(CCNC(=O)CN2CCN(CC(=O)O)CCN(CC(=O)O)CCN(CC(=O)O)CC2)C1=O)C(=O)NCC1CCC(C(=O)NC(Cc2ccc3ccccc3c2)C(=O)NCCCCC(NC(=O)NC(CCC(=O)O)C(=O)O)C(=O)O)CC1 ; O=C(ON1C(=O)CCC1=O)c1ccc(-n2nc(-c3ccccc3O)nc2-c2ccccc2O)cc1]

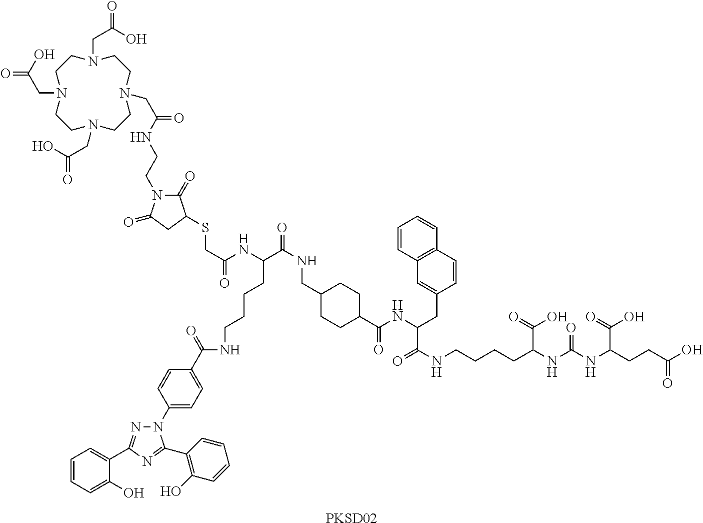 [SMILES: O=C(O)CCC(NC(=O)NC(CCCCNC(=O)C(Cc1ccc2ccccc2c1)NC(=O)C1CCC(CNC(=O)C(CCCCNC(=O)c2ccc(-n3nc(-c4ccccc4O)nc3-c3ccccc3O)cc2)NC(=O)CSC2CC(=O)N(CCNC(=O)CN3CCN(CC(=O)O)CCN(CC(=O)O)CCN(CC(=O)O)CC3)C2=O)CC1)C(=O)O)C(=O)O ; "PKSD02"]

(1) Synthesis of compound 6: a compound 4 (2.33 μmol) was added into a 1.5 mL centrifuge tube, dissolved in DMSO (0.5 mL), raw materials DFX-MAL (4.66 μmol) and PBS solution (0.3 mL) were added, reacted for 12 h at 25° C., purified by HPLC and freeze-dried (at −65° C.) to obtain a white solid, namely compound 6 (2.1 mg, yield 65.01%, purity 98%). HPLC purification included: reverse-phase C18 semi-preparative column (10 mm×250 mm); mobile phase B: acetonitrile+0.1% TFA; gradient elution included: 0 min to 30 min: a volume fraction of the mobile phase B increased from 10% to 90%, and a flow rate of the mobile phase was 3 mL/min. Compound 6: ESI MS [M+H]⁺ for C₆₃H₉₄N₁₃O₂₀S, calculated 1385.57, found 1385.48.

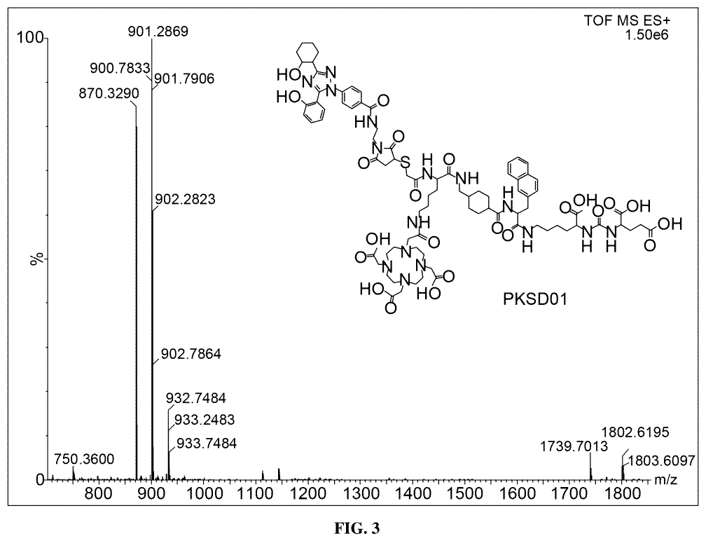

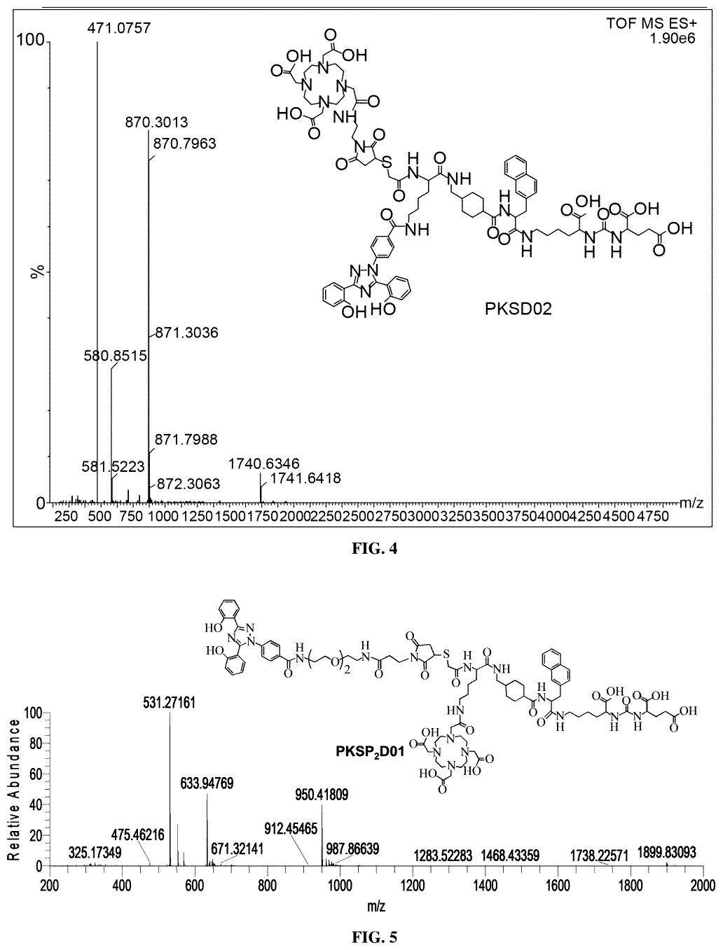

(2) Synthesis of PKSD02: compound 6 (1.52 μmol) was added into a 1.5 mL centrifuge tube, DMSO (0.5 mL), DFX-NHS (3.79 μmol), and DIPEA (4.55 μmol) were added, stirred at 25° C. for 12 h, purified by HPLC and freeze-dried (at −65° C.) to obtain compound PKSD02 (1 mg, yield 37.87%) with a purity of greater than 95% after identification. HPLC purification included: reverse-phase C18 semi-preparative column (10 mm×250 mm); mobile phase A: water+0.1% TFA; mobile phase B: acetonitrile+0.1% TFA; gradient elution included: 0 min to 30 min: a volume fraction of the mobile phase B increased from 10% to 90%, and a flow rate of the mobile phase was 3 mL/min. PKSD02: ESI MS [M+H]⁺ for C₈₄H₁₀₇N₁₆O₂₃S, calculated 1740.92, found 1740.63, as shown in FIG. 4; the HPLC purity analysis is shown in FIG. 7D, and the purity was greater than 95%.

### EXAMPLE 5

### Synthesis of PKSP₂D01

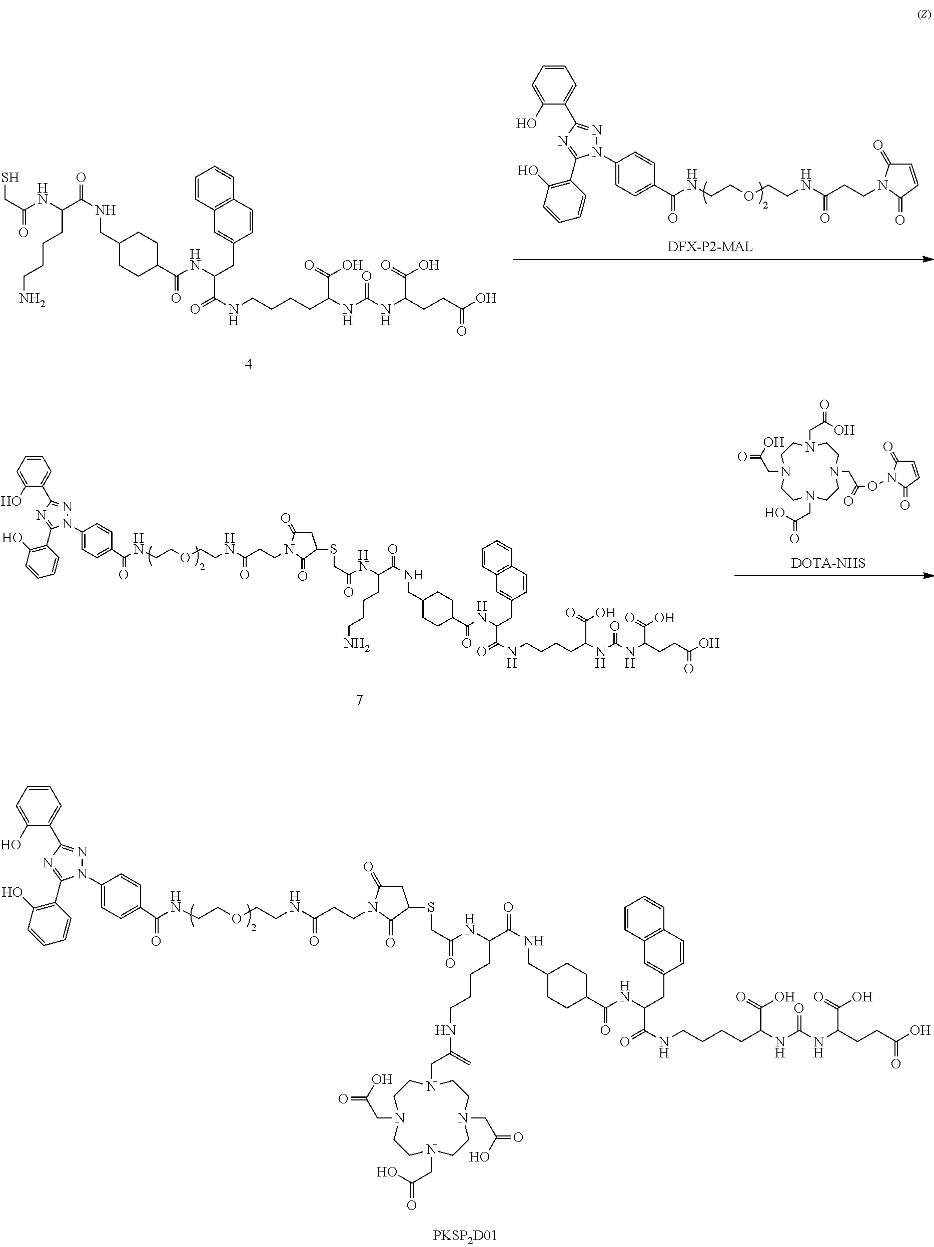

(1) Synthesis of compound 7: a compound 4 (3.5 μmol) and DFX-P₂-MAL (5.24 μmol) were added into a 1.5 mL centrifuge tube, dissolved in DMSO (0.3 mL) and PBS (pH=7.4), reacted for 12 h at 25° C., purified by HPLC and freeze-dried (at −65° C.) to obtain a white solid, namely compound 7 (1.3 mg, yield 24.6%, purity 95%). HPLC purification included: reverse-phase C18 semi-preparative column (10 mm×250 mm); mobile phase A: water+0.1% TFA; mobile phase B: acetonitrile+0.1% TFA; gradient elution included: 0 min to 30 min: a volume fraction of the mobile phase B increased from 10% to 90%, and a flow rate of the mobile phase was 3 mL/min. Compound 7: ESI MS [M+H]⁺ for C₇₅H₉₄N₁₃O₁₉S, calculated 15123.70, found 1513.53.

(2) Synthesis of the PKSP₂D01: compound 7 (0.86 μmol) was added into a 1.5 mL centrifuge tube, DOTA-NHS (1.72 μmol), and DIPEA (4.3 μmol) added, stirred at 25° C. for 12 h, purified by HPLC and freeze-dried (at −65° C.) to obtain compound PKSP₂D01 (1.0 mg, yield 59.9%) with a purity of greater than 95% after identification. HPLC purification included: reverse-phase C18 semi-preparative column (10 mm×250 mm); mobile phase A: water+0.1% TFA; mobile phase B: acetonitrile+0.1% TFA; gradient elution included: 0 min to 30 min: a volume fraction of the mobile phase B increased from 10% to 90%, and a flow rate of the mobile phase was 3 mL/min. PKSP₂D01: ESI MS [M+H]⁺ for C₉₁H₁₂₀N₁₇O₂₆S, calculated 1900.11, found 1899.83, as shown in FIG. 5; the HPLC purity analysis is shown in FIG. 7E, and the purity was greater than 95%.

### EXAMPLE 6

### Radiolabeling Procedure of Gd

Compound PKND01 and gadolinium chloride hexahydrate (GdCl₃·6H₂O) were dissolved in a mixed solvent (DMSO: H₂O at a volume ratio=1:1) at a molar ratio of 1:2, the resulting mixture was adjusted to a pH value of 6.0 with a KOH solution, heated to allow a reaction at 60° C. for 12 h with shaking, and the resulting product was purified by HPLC to obtain a nuclide-targeted probe Gd-PKND01; where compound PKND01 and the mixed solvent were at a dosage ratio of 1 mg:1 mL.

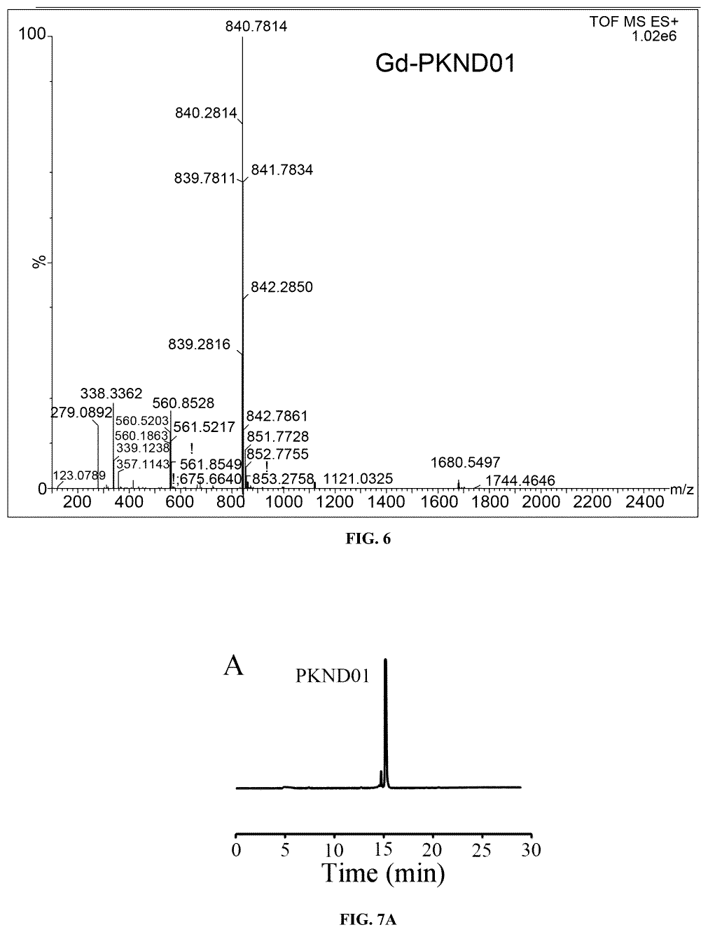

HPLC purification included: reverse-phase C18 analytical column (4.6 mm×250 mm); mobile phase A: water+0.1% TFA; mobile phase B: acetonitrile+0.1% TFA; gradient elution included: 0 min to 30 min: a volume fraction of the mobile phase B increased from 10% to 90%, and a flow rate of the mobile phase was 1 mL/min. The mass spectrometry identification of Gd-PKND01 and the HPLC identification of chemical purity are shown in FIG. 6 and FIG. 8, respectively. ESI MS [M+H]⁺ for C₇₆H₉₄GdN₁₄O₂₀, calculated 1680.91, found 1680.55.

### EXAMPLE 7

### Radiolabeling Procedure of⁶⁸Ga

Wet method: 370 MBq of a ⁶⁸GaCl₃ hydrochloric acid solution (eluted from a germanium-gallium generator) was added into an acetic acid-acetate solution containing 0.5 mL of the Glu-urea compounds (20 μg) prepared in Examples 1 to 5 separately, reacted at 90° C. for 20 min and then cooled to room temperature; an obtained product was diluted with physiological saline or water for injection and sterile-filtered to obtain an injection of the nuclide-targeted probe (a concentration of the nuclide-targeted probe was 110 MBq/mL).

Freeze-drying method: a certain amount of the buffer (sodium acetate buffer, pH=5.5, 0.4 mL) and 370 MBq of ⁶⁸GaCl₃ eluate (eluted from a germanium-gallium generator) were added into a freeze-dried kit containing 20 μg of the Glu-urea compound, reacted at 90° C. for 20 min and then cooled to room temperature; an obtained product was diluted with physiological saline or water for injection and sterile-filtered to obtain an injection of the nuclide-targeted probe (a concentration of the nuclide-targeted probe was 110 MBq/mL).

If the radiochemical purity was less than 95%, purification was conducted through a C18separation cartridge to remove unreacted ⁶⁸Ga³⁺. Purification of the C18 separation cartridge included: the Sep-Pak C18 separation cartridge was used to allow activation and elution through 10 mL of absolute ethanol and 10 mL of water in sequence. A labeling solution was labeled with 10 mL of water and loaded onto the separation column. The separation column was rinsed with water to remove unreacted ⁶⁸Ga ions, and then eluted with ethanol solution to obtain a ⁶⁸Ga-labeled probe.

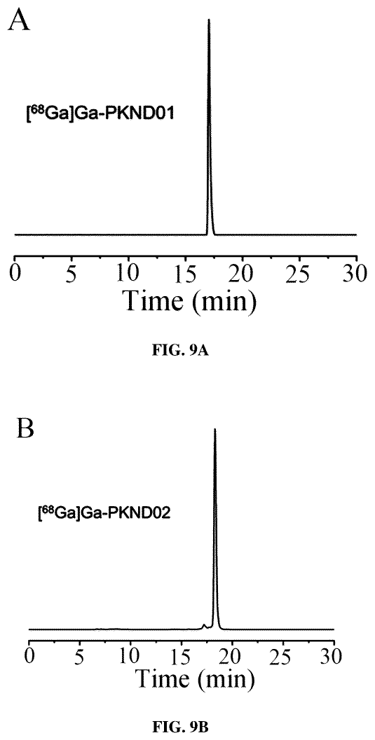

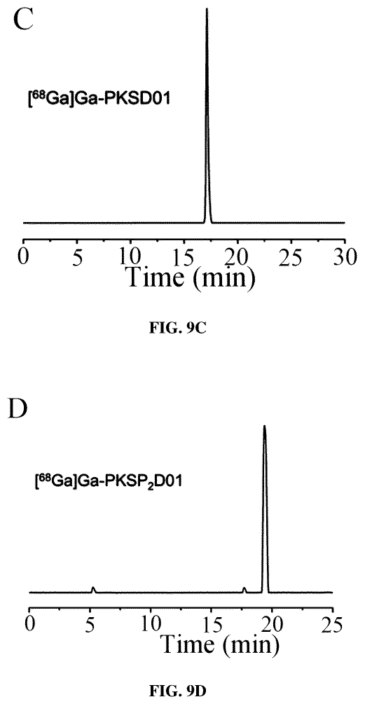

The HPLC identification results of the radiochemical purity of [⁶⁸Ga]Ga-PKND01, [⁶⁸Ga]Ga-PKND02, [⁶⁸Ga]Ga-PKSD01, and [⁶⁸Ga]Ga-PKSP₂D01 are shown in FIG. 9A, FIG. 9B, FIG. 9C, and FIG. 9D, respectively, and the radiochemical purity of each nuclide-targeted probe was greater than 95%.

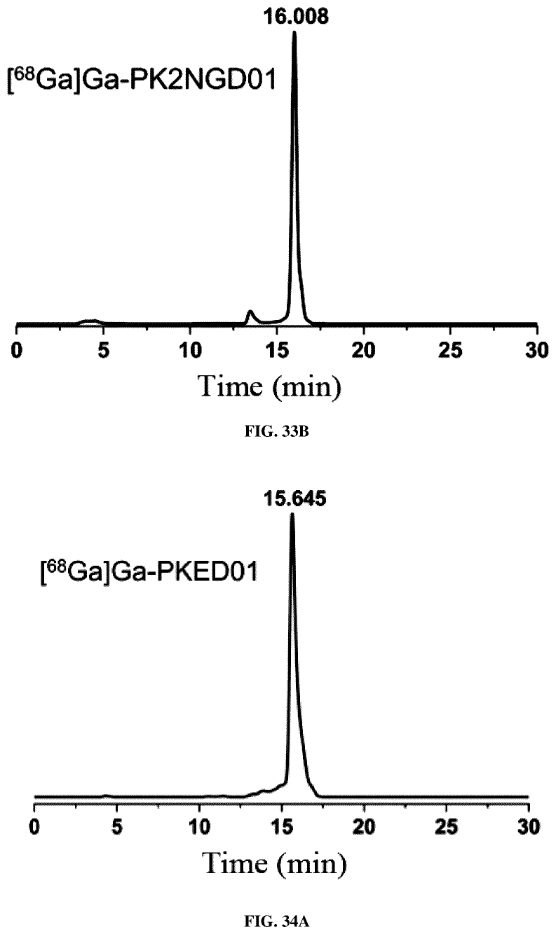

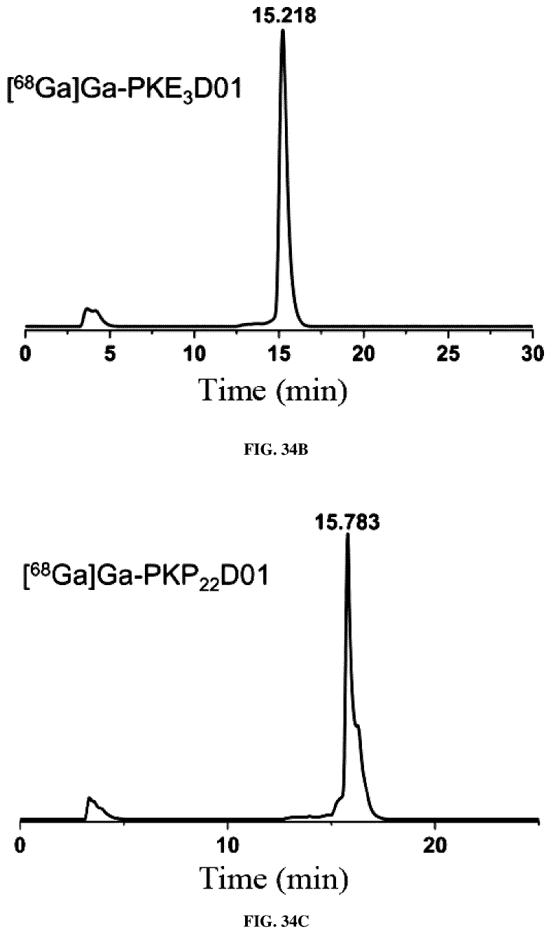

The HPLC identification patterns of radiochemical purity of probes [⁶⁸Ga]Ga-PK2ND01 and [⁶⁸Ga]Ga-PK2NGD01 are shown in FIG. 33A and FIG. 33B, respectively; and the HPLC identification patterns of radiochemical purity of probes [⁶⁸Ga]Ga-PKED01, [⁶⁸Ga]Ga-PKE₃D01, and [⁶⁸Ga]Ga-PKP₂₂D01 are shown in FIG. 34A, FIG. 34B, and FIG. 34C, respectively.

### EXAMPLE 8

### Radiolabeling Procedure of ¹⁷⁷Lu

Wet method: 370 MBq of a ¹⁷⁷LuCl₃ solution was added into an acetic acid-acetate solution containing 0.2 mL of the compounds (20 μg) prepared in Examples 1 to 3 separately, reacted at 90° C. for 20 min and then cooled to room temperature; an obtained product was diluted with physiological saline or water for injection and sterile-filtered to obtain an injection of the nuclide-targeted probe (a concentration of the nuclide-targeted probe was 110 MBq/mL).

Freeze-drying method: a certain amount of the buffer (sodium acetate buffer, pH=5.5, 0.2 mL) and 370 MBq of ¹⁷⁷LuCl₃ solution were added into a freeze-dried kit containing 20 μg of the Glu-urea compound, mixed well and reacted at 90° C. for 20 min and then cooled to room temperature; an obtained product was diluted with physiological saline or water for injection and sterile-filtered to obtain an injection of the nuclide-targeted probe (a concentration of the nuclide-targeted probe was 110 MBq/mL).

If the radiochemical purity was less than 95%, purification was conducted through a C18separation cartridge to remove unreacted ¹⁷⁷Lu³⁺. Purification of the C18 separation cartridge included: the Sep-Pak C18 separation cartridge was used to allow the activation and elution through 10 mL of absolute ethanol and 10 mL of water in sequence. A labeling solution was labeled with 10 mL of water and loaded onto the separation column. The separation column was rinsed with water to remove unreacted ¹⁷⁷Lu ions, and then eluted with ethanol solution to obtain a ¹⁷⁷Lu-labeled probe.

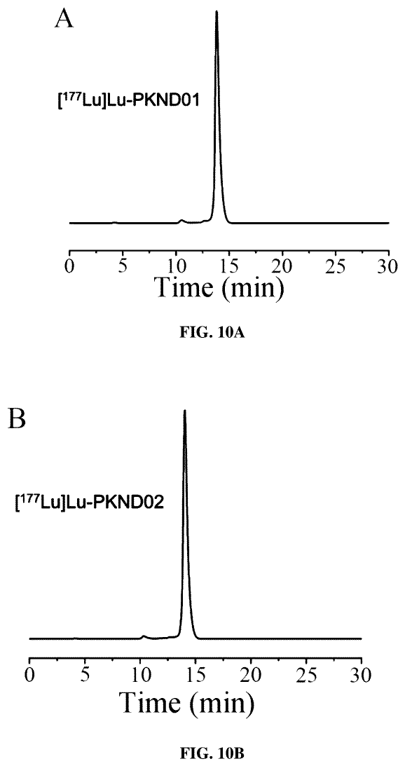

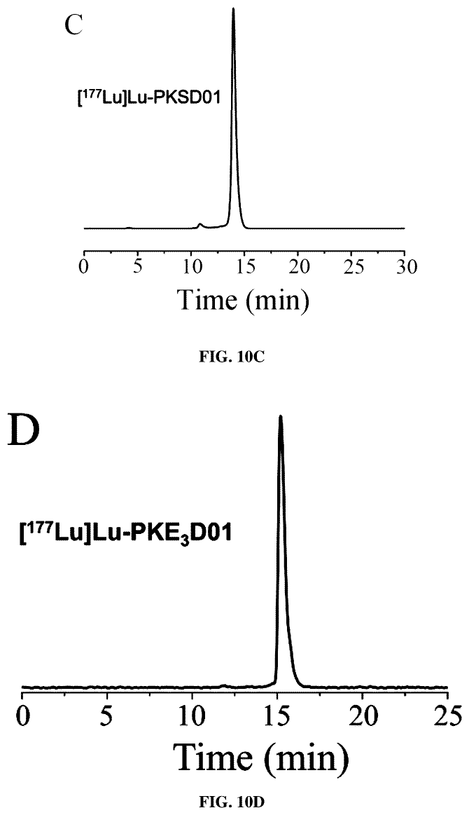

The HPLC identification results of the radiochemical purity of [¹⁷⁷Lu]Lu-PKND01, [¹⁷⁷Lu]Lu-PKND02, [¹⁷⁷Lu]Lu-PKSD01, and [¹⁷⁷Lu]Lu-PKE₃D01 are shown in FIG. 10A, FIG. 10B, FIG. 10C, and FIG. 10D, respectively, and the radiochemical purity of each nuclide-targeted probe was greater than 95%.

### TEST EXAMPLE 1

### Evaluation of Stability and Lipid-water Distribution Properties

1. Injection solution stability test: the nuclide-targeted probe diluted in physiological saline was placed at room temperature for different times, and samples were taken for analysis by HPLC. HPLC purification included: reverse-phase C18 analytical column (4.6 mm×250 mm); mobile phase A: water+0.1% TFA; mobile phase B: acetonitrile+0.1% TFA; gradient elution included: 0 min to 30min: a volume fraction of the mobile phase B increased from 10% to 90%, and a flow rate of the mobile phase was 1 mL/min.

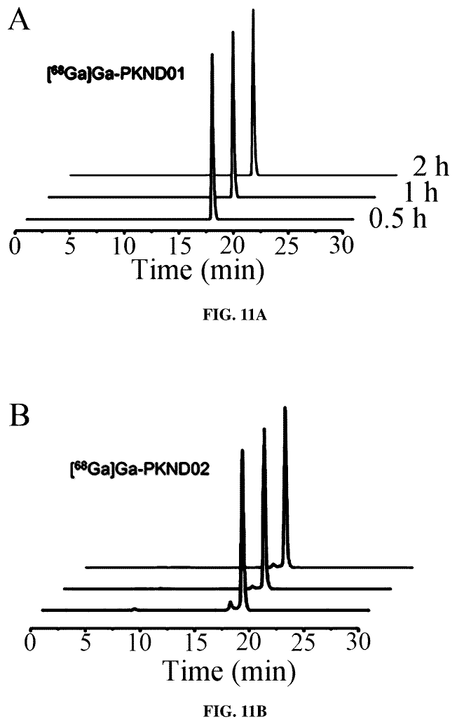

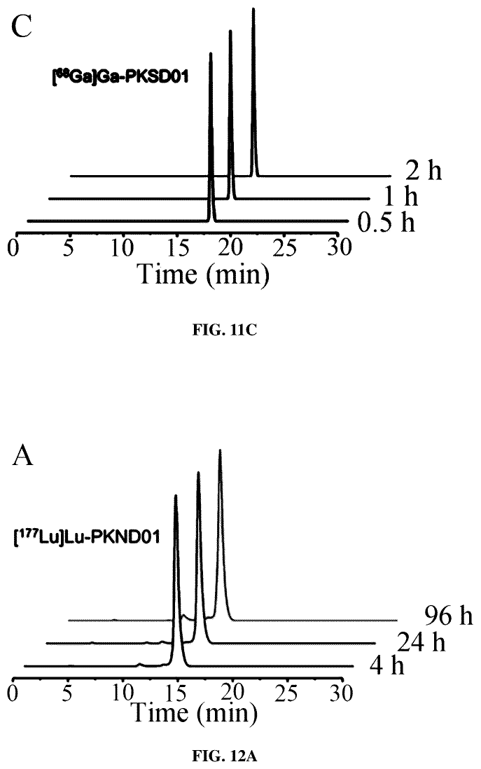

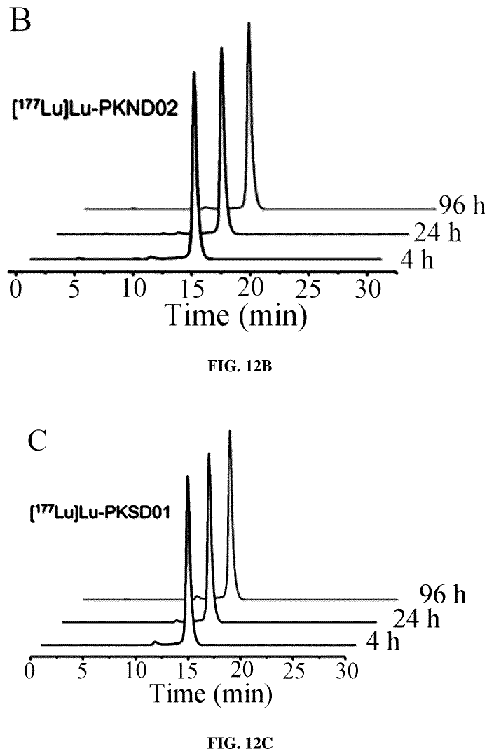

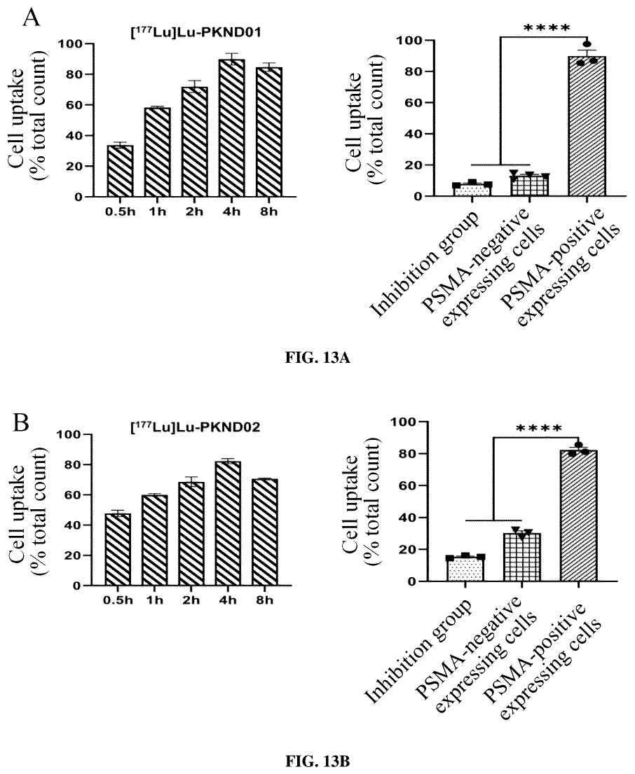

The HPLC identification results of the stability of [⁶⁸Ga]Ga-PKND01, [⁶⁸Ga]Ga-PKND02, and [⁶⁸Ga]Ga-PKSD01 are shown in FIG. 11A, FIG. 11B, and FIG. 11C, respectively; the HPLC identification results of the stability of [¹⁷⁷Lu]Lu-PKND01, [¹⁷⁷Lu]Lu-PKND02, and [¹⁷⁷Lu]Lu-PKSD01 are shown in FIG. 12A, FIG. 12B, and FIG. 12C, respectively. As shown in FIG. 11A-FIG. 11C and FIG. 12A-FIG. 12C, the radiochemical purity of each nuclide-targeted probe at the tested time point was greater than 95%, indicating that its properties were stable in the specified solution.

2. Determination of Octanol/Water Partition Coefficient (log P):

100 μL of a diluted solution containing the nuclide-targeted probe was added into a centrifuge tube containing a mixture of 2.9 mL of PBS and 3 mL of n-octanol, vortex-shaken for 3 min, and centrifuged at 10,000 rpm for 3 min. 100 μL of liquid from each of obtained aqueous phase and n-octanol phase were collected to measure the radioactivity count by γ-counter. The experiment was repeated three times and an average value was taken. A calculation formula of the log P was:

P=I_{organic phase}/I_{aqueous phase}; where

I_{organic phase} represented the radioactivity count measured in the organic phase, and I_{aqueous phase} represents the radioactivity count measured in the aqueous phase. Through calculation, the Octanol/Water Partition Coefficient of each radioactively-labeled targeting probe was finally determined. The results are shown in Table 1, the tested nuclide-targeted probes were water-soluble.

TABLE 1
Octanol/Water Partition Coefficient of nuclide-targeted probe

| Probe | Log P |
|---|---|
| [¹⁷⁷Lu]Lu-PKND01 | −2.31 ± 0.16 |
| [¹⁷⁷Lu]Lu-PKND02 | −2.07 ± 0.06 |
| [¹⁷⁷Lu]Lu-PKSD01 | −2.21 ± 0.04 |
| [¹⁷⁷Lu]Lu-PKSD02 | −2.09 ± 0.03 |
| [¹⁷⁷Lu]Lu-PKE₃D01 | −2.20 ± 0.08 |

As shown in Table 1, the above 5 ¹¹⁷/Lu-labeled probes were water-soluble. This indicated that the ¹¹⁷Lu-labeled probe prepared in the present disclosure could avoid non-specific uptake of radioactivity in normal tissues through renal metabolism.

### TEST EXAMPLE 2

### Cellular Uptake and Inhibition Experiments

PC3 PIP cells with high PSMA expression and PC3 flu cells with negative expression were placed in a twenty-four-well plate containing a medium (fetal bovine serum and double antibodies) (the number of cells was counted on a cell counting plate, about 200,000 cells/well) and then cultured for 24 h. At the beginning of the uptake experiment, the original medium was removed, washed twice with PBS (500 μL), and the PBS was removed; an equal amount of the probe to be tested that had been diluted with medium was added into each well, and incubated at 37° C. for 0.5 h, 1 h, 2 h, 4 h, and 8 h separately; after the incubation at each time point, the medium was removed, a sodium hydroxide (NaOH) solution (500 μL, 1 M) was added to each well to lyse the cells; after 5 min, the lysed cells were placed into a disposable centrifuge tube for measurement of radioactivity count; the radioactivity count was divided by a total amount of radioactivity added to obtain the percentage of cellular uptake.

In order to examine the targeting specificity of the probe PSMA, this study also set up an inhibitory group: before adding the nuclide-labeled probe, an appropriate amount of inhibitor PSMA617 was added into the cells in each well and incubated at 37° C. for 2 h and 4 h separately; after the incubation, the radioactive medium was removed, and a sodium hydroxide solution (500 μL, 1 M) was added into each well to lyse the cells; after 5 min, the lysed cells were placed into a disposable centrifuge tube for measurement of radioactivity count; the radioactivity count was divided by a total amount of radioactivity added to obtain the percentage of cellular uptake.

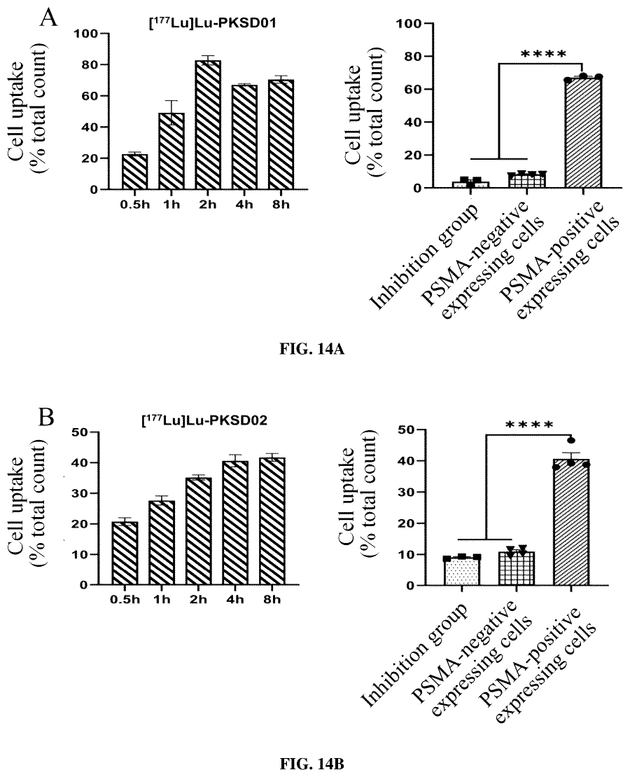

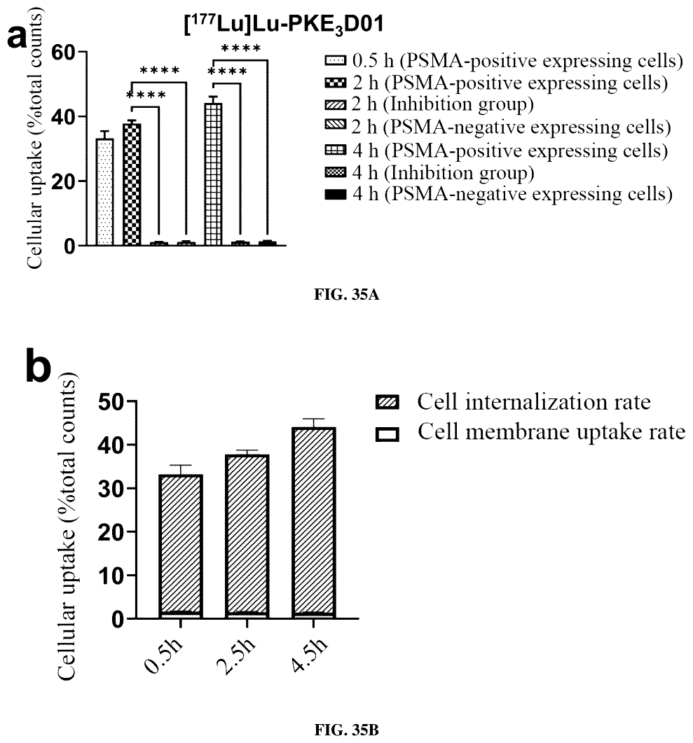

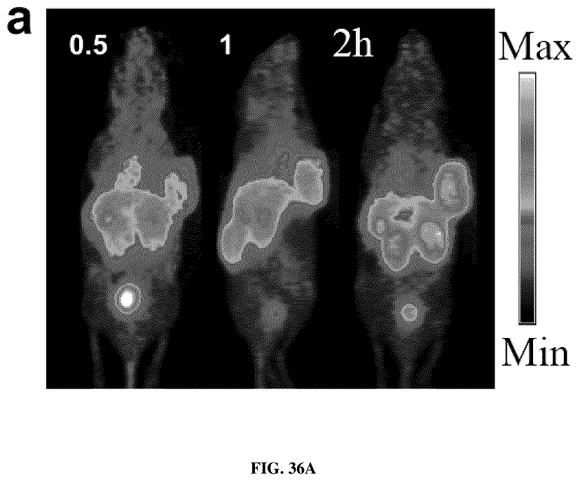

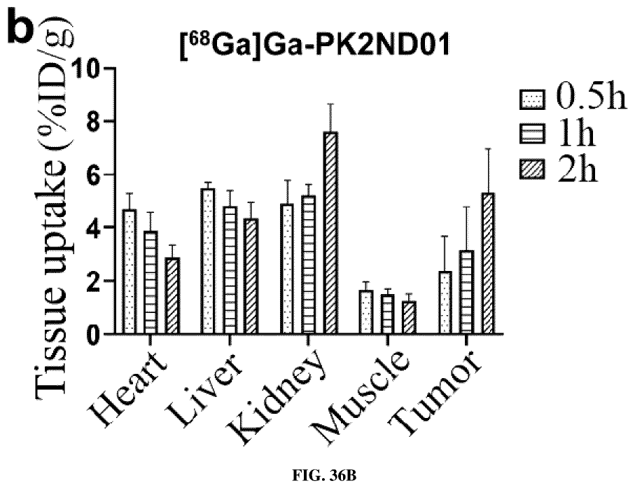

The cellular uptake and inhibition results of [¹⁷⁷Lu]Lu-PKND01, [¹⁷⁷Lu]Lu-PKND02, [¹⁷⁷Lu]Lu-PKSD01, and [¹⁷⁷Lu]Lu-PKSD02 are shown in FIG. 13A-FIG. 13B to FIG. 14A-FIG. 14B. Taking the 4 h results as an example, each nuclide-targeted probe had significant uptake in PSMA-positive cells, while the uptake in negative cells was significantly reduced. The uptake of each nuclide-targeted probe in PSMA-positive expressing cells was inhibited by PSMA617, indicating that the nuclide-targeted probe provided by the present disclosure was specific for targeting PSMA protein. The cellular uptake and inhibition results of probe [¹⁷⁷Lu]Lu-PKE₃D01 are shown in FIG. 35A and FIG. 35B. The uptake of the nuclide-targeting probes in PSMA-positive cells at each time point was significantly higher than that in the PSMA-negative cell group and the inhibition group. In addition, the internalization rate of [¹⁷⁷Lu]Lu-PKE₃D01 in PSMA-positive expression cells was significantly higher than that of membrane binding rate, indicating that the probe could enter the cells to played a role.

### TEST EXAMPLE 3

### PET Imaging Experiment

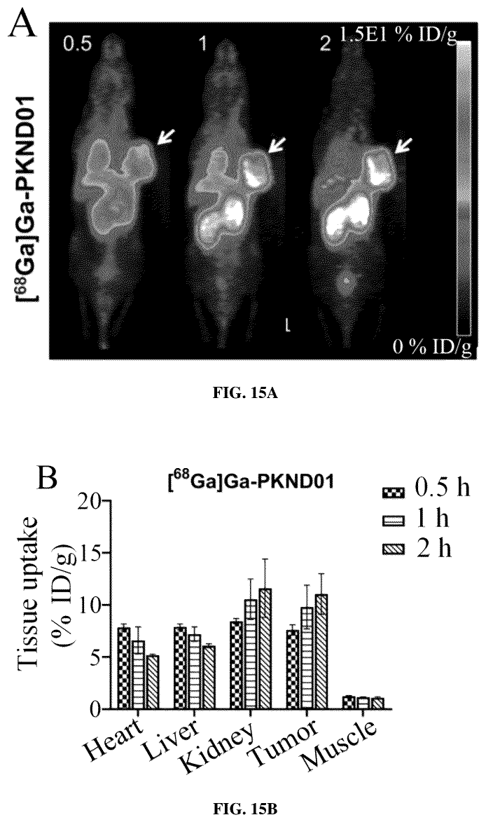

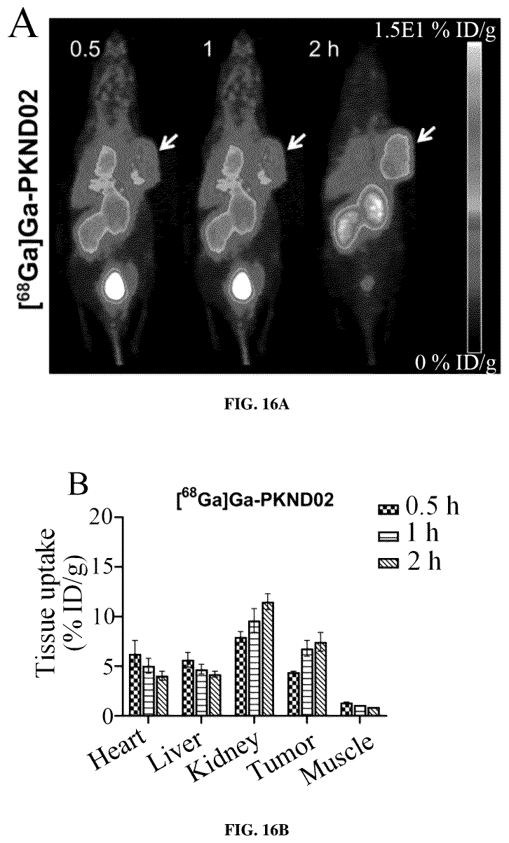

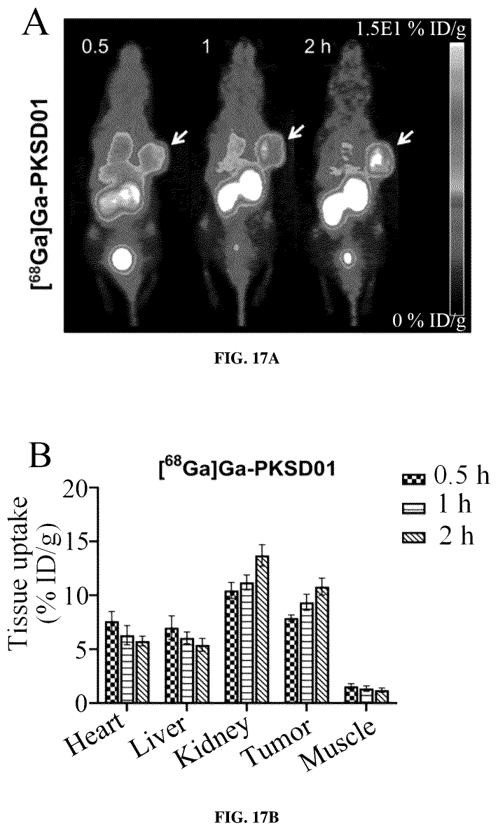

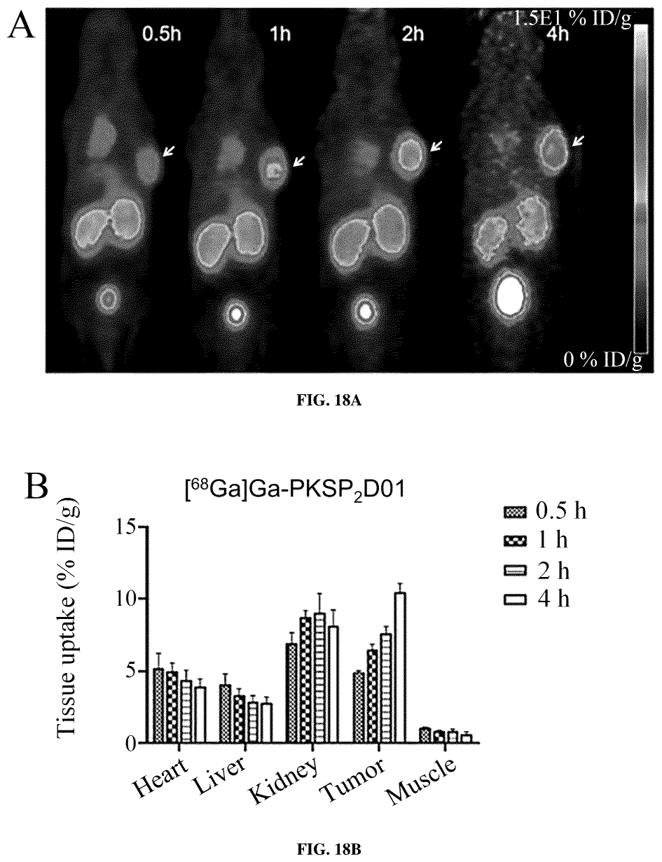

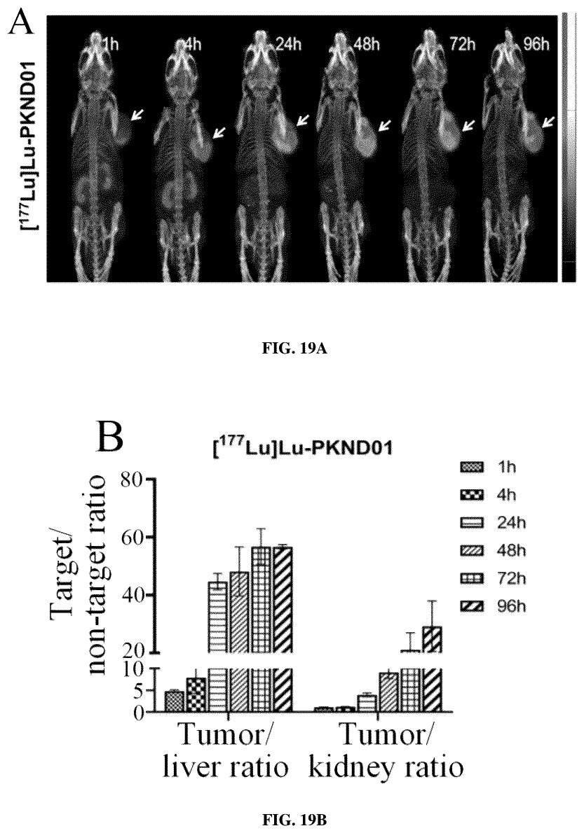

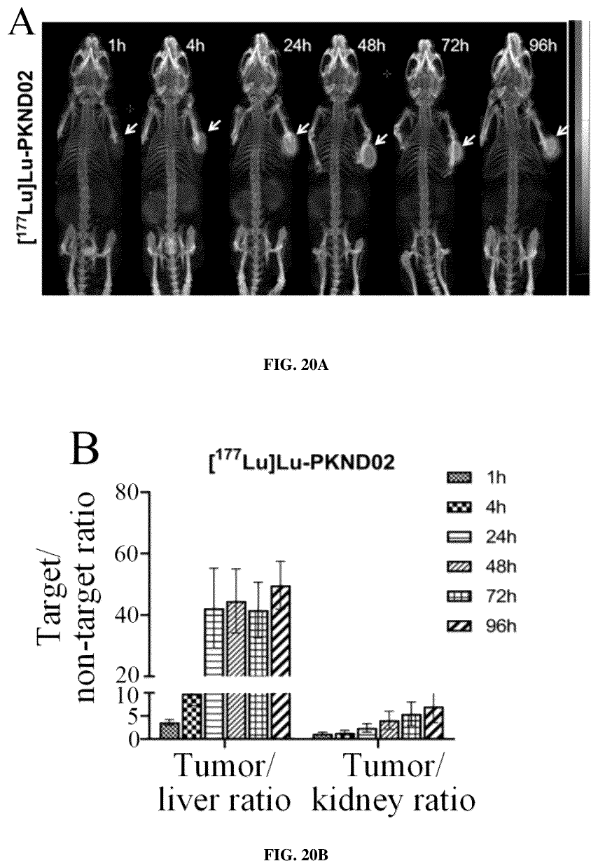

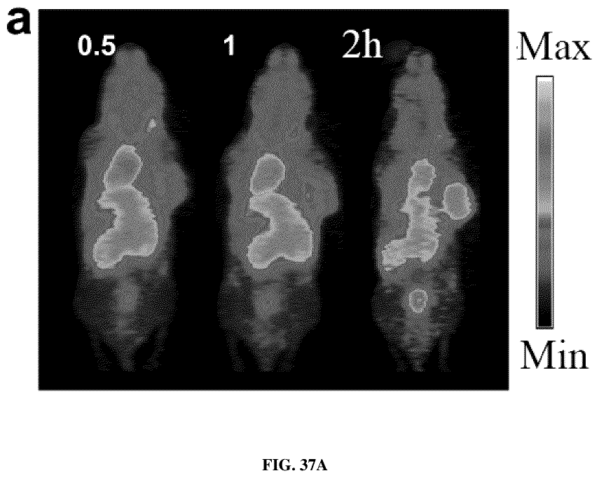

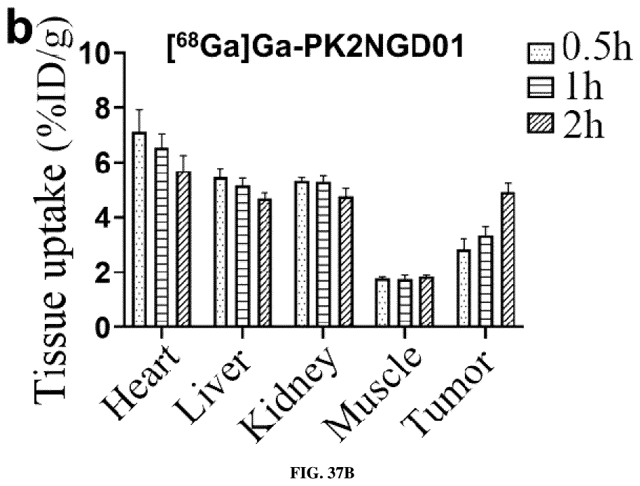

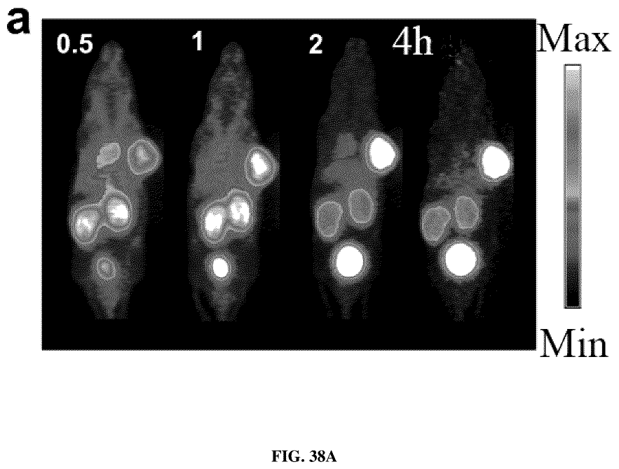

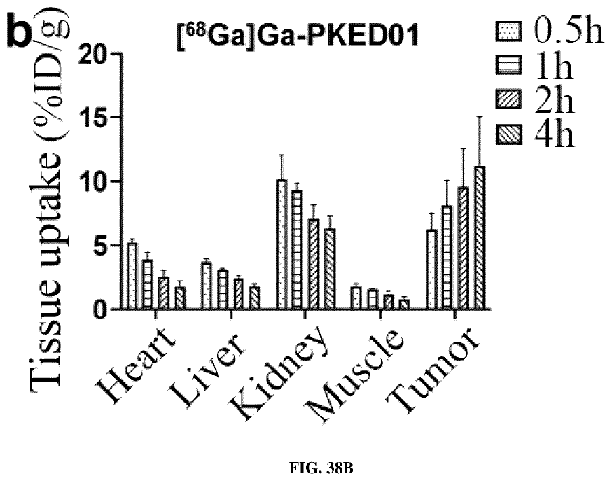

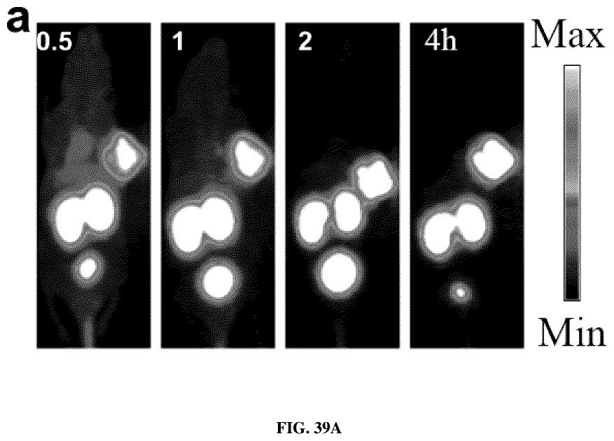

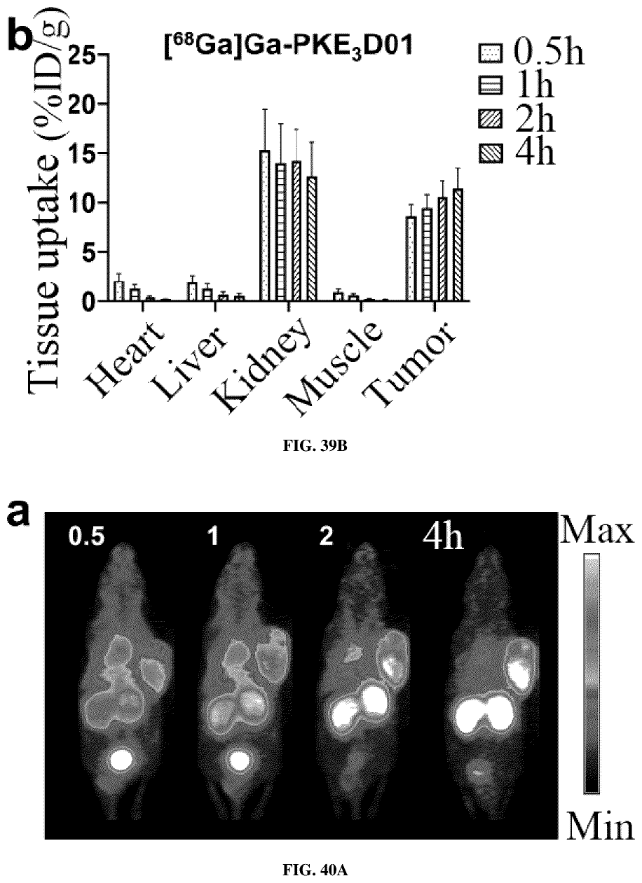

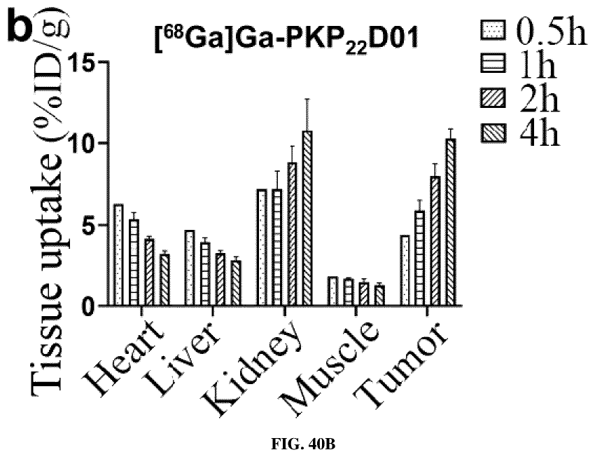

The ⁶⁸Ga-labeled probe prepared in the example with a radiochemical purity of greater than 95% was diluted with physiological saline, and 0.2 mL (1 MBq) of an obtained injection was injected through the tail vein of the PC3 PIP model mouse. MicroPET imaging was conducted at different time points, a region of interest (ROI) was drawn on the image, and a probe distribution value was obtained through calculation. The PET imaging results of [⁶⁸Ga]Ga-PKND01, [⁶⁸Ga]Ga-PKND02, [⁶⁸Ga]Ga-PKSD01, [⁶⁸Ga]Ga-PKSP2D01, [⁶⁸Ga]Ga-PK2ND01, [⁶⁸Ga]Ga-PK2NGD01, [⁶⁸Ga]Ga-PKED01, [⁶⁸Ga]Ga-PKE₃D01, and [⁶⁸Ga]Ga-PKP₂₂D01 are shown in FIG. 15A-FIG. 15B, FIG. 16A-FIG. 16B, FIG. 17A-FIG. 17B, FIG. 18A-FIG. 18B, FIG. 36A-FIG. 36B, FIG. 37A-FIG. 37B, FIG. 38A-FIG. 38B, FIG. 39A-FIG. 39B, and FIG. 40A-FIG. 40B, respectively. It can be known that the nuclide-targeted probe had a high uptake at the tumor site. There were higher radioactive signals in the bladder and kidneys, which indicated that the nuclide-targeted probe was excreted in the urine. Absolute uptake and target/non-target ratios in the tumors increased significantly over time.

### TEST EXAMPLE 4

### SPECT Imaging Experiment

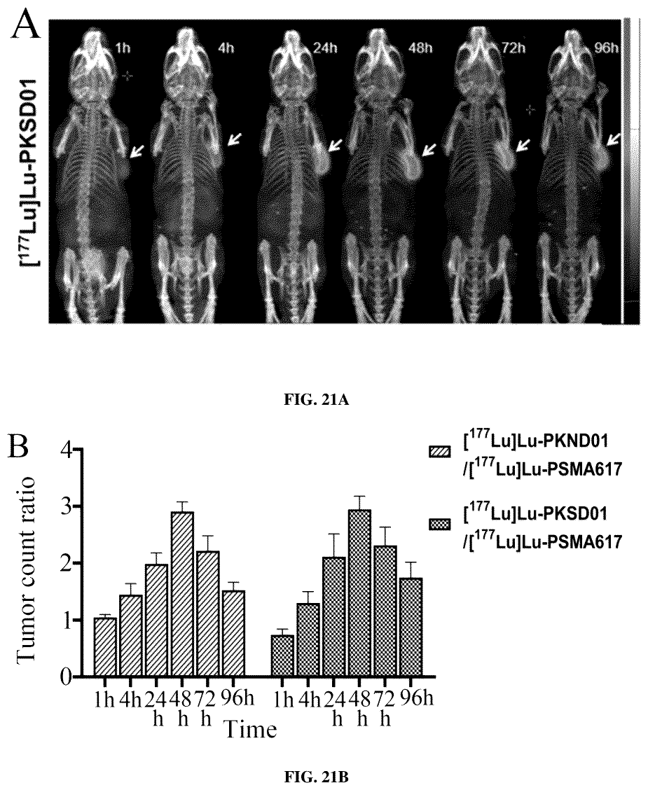

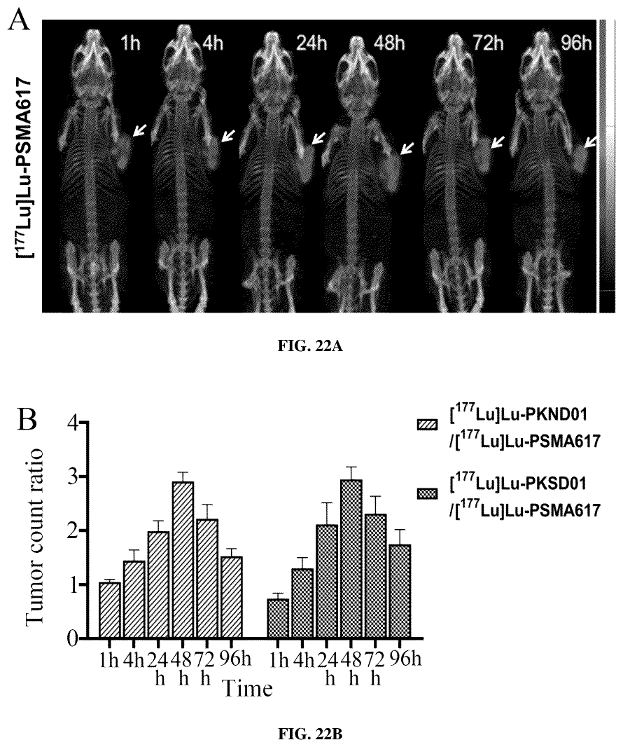

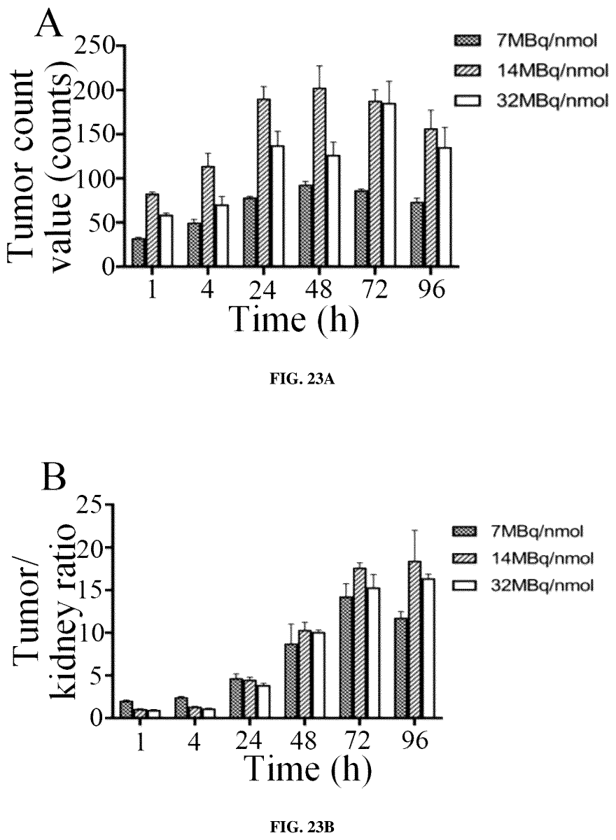

Tumor-bearing model mice were injected with 14 MBq of ¹⁷⁷Lu-labeled probe through the tail vein, and static SPECT scanning imaging was conducted at different time points after injection assisted with CT scanning positioning. After the imaging was completed, the image was reconstructed and the ROI in the mouse image was outlined to obtain the radioactivity count value. The target/non-target ratio of the probe distribution was calculated. The SPECT imaging results of [¹⁷⁷Lu]Lu-PKND01, [¹⁷⁷Lu]Lu-PKND02, and [¹⁷⁷Lu]Lu-PKSD01 are shown in FIG. 19A-FIG. 19B, FIG. 20A-FIG. 20B, and FIG. 21A-FIG. 21B, respectively. The SPECT imaging results of the control group [¹⁷⁷Lu]Lu-PSMA617 are shown in FIG. 22A-FIG. 22B. Within the monitoring time range, the enrichment of [¹⁷⁷Lu]Lu-PKND01, [¹⁷⁷Lu]Lu-PKND02, and [¹⁷⁷Lu]Lu-PKSD01 nuclide-targeted probes in the tumor site was significantly higher than that of the control group [¹⁷⁷Lu]Lu-PSMA617, with high imaging contrast and clear lesion outline. This proved that the nuclide-targeted probe provided by the present disclosure had a desirable tumor uptake effect. Over time, the radioactive background in the blood pool and other normal organs was gradually cleared, and the target/non-target ratio continued to increase. The above data showed that the nuclide-targeted probe provided by the present disclosure had a better nuclide-targeted therapeutic potential than [¹⁷⁷Lu]Lu-PSMA617.

FIG. 23A-FIG. 23B show the SPECT imaging tumor uptake results (a) and tumor/kidney ratio (b) of [¹⁷⁷Lu]Lu-PKND01 with different specific activities. SPECT imaging experiments were conducted to compare the distribution of different specific activities of [¹⁷⁷Lu]Lu-PKND01 (by adding different masses of PKND01 in the labeling solution) in tumor-bearing mice. As shown in FIG. 23A-FIG. 23B, when the specific activity was 14 MBq/nmol, the [¹⁷⁷Lu]Lu-PKND01 had both desirable tumor uptake and high tumor/kidney ratio.

### TEST EXAMPLE 5

### MRI Imaging Experiment

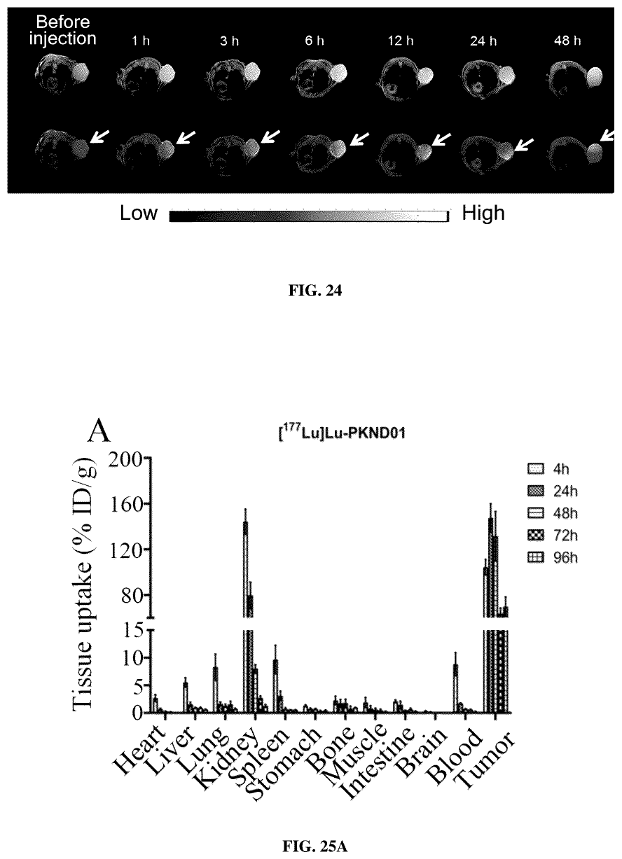

The Gd-PKND01 compound was dissolved in PBS buffer and injected into tumor-bearing mice through the tail vein at a dose of 11.9 μmol/kg. MRI imaging was conducted at 1 h, 3 h, 6 h, 12 h, 24 h, and 48 h separately after injection. As shown in FIG. 24, the area pointed by the arrow was the location of the tumor. Compared with before injection, the PC3-pip tumor showed a gradually enhanced signal after injection, indicating that the nuclide-targeted probe provided by the present disclosure had a better enrichment effect at the tumor site.

### TEST EXAMPLE 6

### Biodistribution Experiment

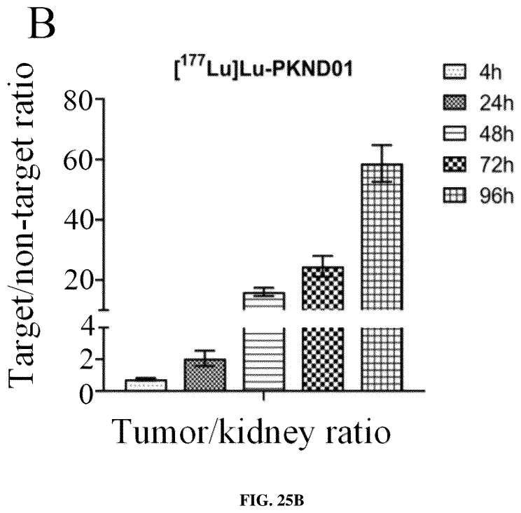

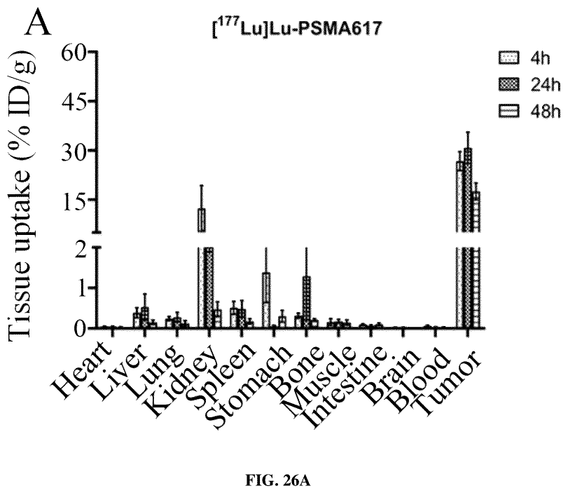

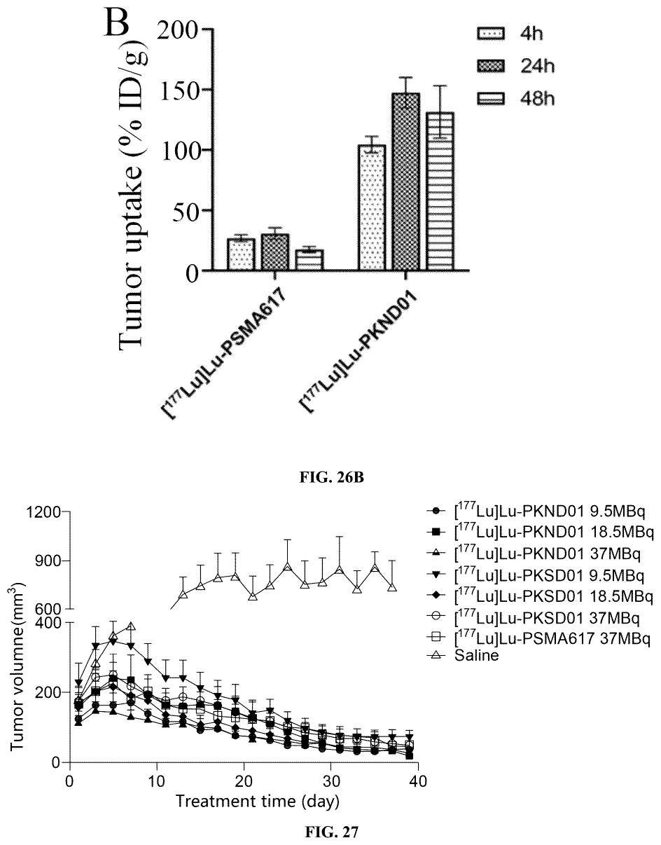

Tumor-bearing mice were injected with 1.5 MBq of [¹⁷⁷Lu]Lu-PKND01 or [¹⁷⁷Lu]Lu-PSMA617 separately through the tail vein. The mice were sacrificed at different time points after injection, and tumor and other organ tissue samples were obtained by dissection, weighed, and radioactivity counts were measured with a γ counter. Results were expressed as percent absorbed dose per gram of tissue or organ (% ID/g). The biodistribution results of [¹⁷⁷Lu]Lu-PKND01 and [¹⁷⁷Lu]Lu-PSMA617 are shown in FIG. 25A-FIG. 25B and FIG. 26A-FIG. 26B, respectively. 4 h after injection, the tumor uptake of [¹⁷⁷Lu]Lu-PKND01 was greater than 80% ID/g. 24 h after injection, the tumor uptake reached 150% ID/g and remained at 60% ID/g at 96 h. It was seen that compared with [¹⁷⁷Lu]Lu-PSMA617, the nuclide-targeted probe [¹⁷⁷Lu]Lu-PKND01 provided by the present disclosure had significantly enhanced tumor uptake and prolonged residence time.

### TEST EXAMPLE 7

### Nuclide-targeted Therapy Experiment

The tumor-bearing mice were divided into an experimental group, a [¹⁷⁷Lu]Lu-PSMA617 control group, and a physiological saline group, with 6 to 8 mice in each group. Each mouse in the experimental group was injected with different doses of [¹⁷⁷Lu]Lu-PKND01 or [¹⁷⁷Lu]Lu-PKSD01 through the tail vein; each mouse in the [¹⁷⁷Lu]Lu-PSMA617 control group was injected with 37 MBq of [¹⁷⁷Lu]Lu-PSMA617 through the tail vein; each mouse in the physiological saline group was injected with a same volume of physiological saline into the tail vein, and changes in tumor size and body weight were monitored every day. The treatment results are shown in FIG. 27. Compared with the physiological saline group, the tumor volume of the experimental group decreased significantly over time, and all doses of [¹⁷⁷Lu]Lu-PKND01 had obvious tumor treatment effects. Moreover, low-dose [¹⁷⁷Lu]Lu-PKND01 or [¹⁷⁷Lu]Lu-PKSD01 (9.5 MBq) had a similar efficacy compared with high-dose [¹⁷⁷Lu]Lu-PSMA617 (37 MBq), indicating that the nuclide-targeted probe provided by the present disclosure showed a desirable application potential.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A glutamic acid (Glu)-urea compound, wherein the Glu-urea compound has a structure shown in formula I-1 or formula I-2: 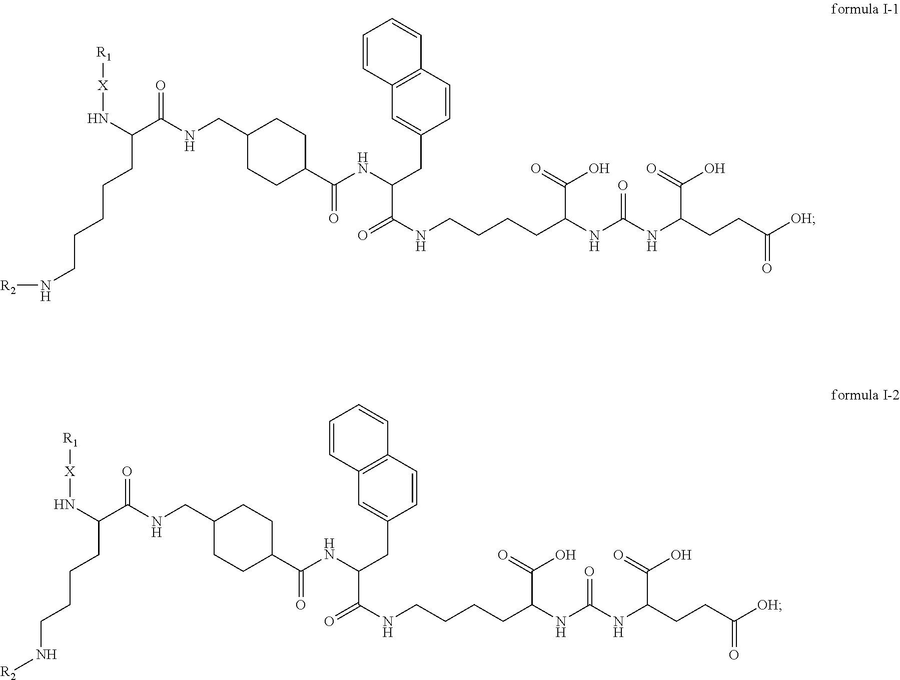
wherein one of R₁ and R₂ is selected from the group consisting of 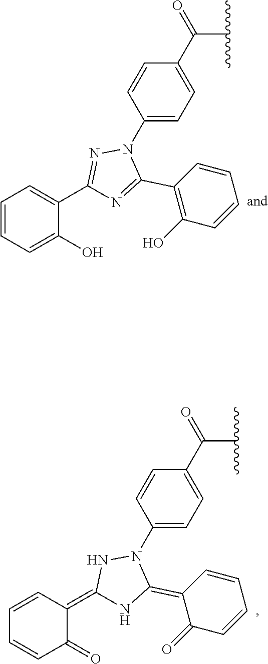 [SMILES: *C(=O)c1ccc(-n2nc(-c3ccccc3O)nc2-c2ccccc2O)cc1 ; *C(=O)c1ccc(N2N/C(=C3\C=CC=CC3=O)N/C2=C2/C=CC=CC2=O)cc1]
while the other is a group to be labeled, and the group to be labeled is one selected from the group consisting of: 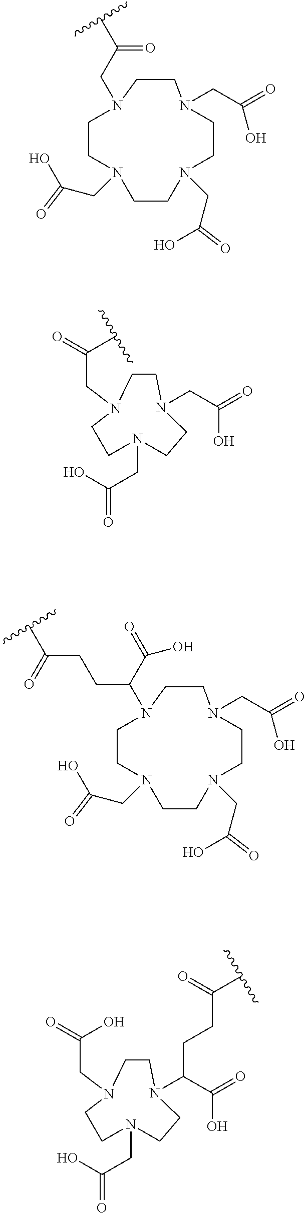 [SMILES: *C(=O)CN1CCN(CC(=O)O)CCN(CC(=O)O)CCN(CC(=O)O)CC1 ; *C(=O)CN1CCN(CC(=O)O)CCN(CC(=O)O)CC1 ; *C(=O)CCC(C(=O)O)N1CCN(CC(=O)O)CCN(CC(=O)O)CCN(CC(=O)O)CC1 ; *C(=O)CCC(C(=O)O)N1CCN(CC(=O)O)CCN(CC(=O)O)CC1] 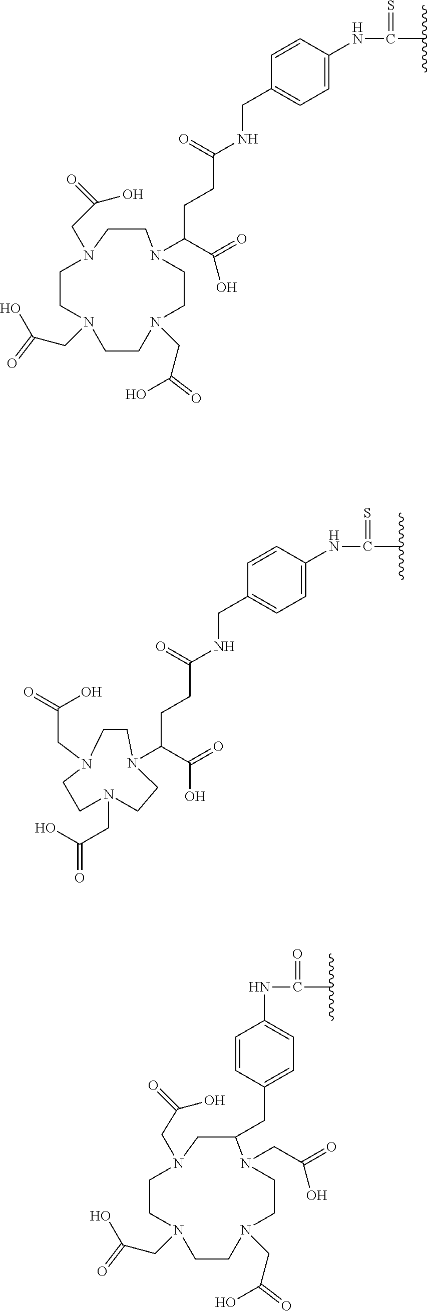 [SMILES: *C(=S)Nc1ccc(CNC(=O)CCC(C(=O)O)N2CCN(CC(=O)O)CCN(CC(=O)O)CCN(CC(=O)O)CC2)cc1 ; *C(=S)Nc1ccc(CNC(=O)CCC(C(=O)O)N2CCN(CC(=O)O)CCN(CC(=O)O)CC2)cc1 ; *C(=O)Nc1ccc(CC2CN(CC(=O)O)CCN(CC(=O)O)CCN(CC(=O)O)CCN2CC(=O)O)cc1] 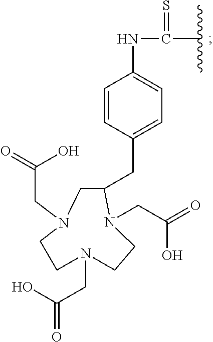 [SMILES: *C(=S)Nc1ccc(CC2CN(CC(=O)O)CCN(CC(=O)O)CCN2CC(=O)O)cc1]
X is present or absent, and X is selected from the group consisting of linking group 1 and linking group 2 when X is present;
linking group 1 is one selected from the group consisting of: 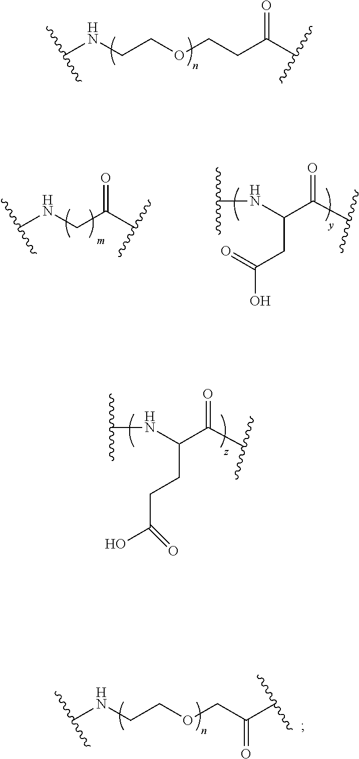
linking group 2 is one selected from the group consisting of: 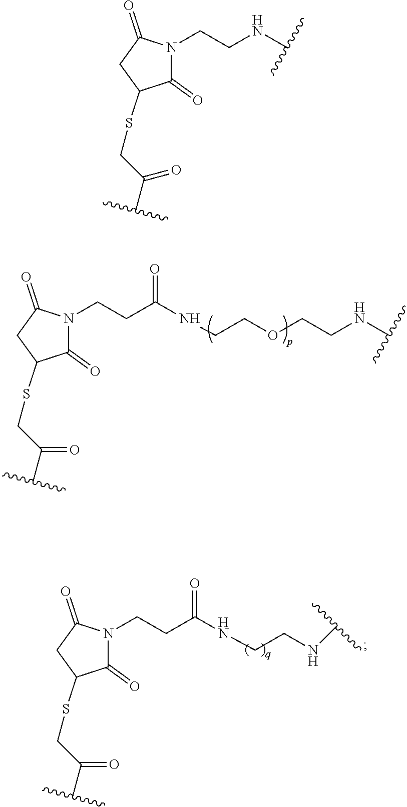
n, m, y, z, p, and q independently are an integer of 0 to 10.

2. A preparation method of the Glu-urea compound according to claim 1, wherein
(i) when X is absent or is linking group 1, the preparation method comprises the following steps:
subjecting Polypeptide compound 1 to first substitution with an R₁ active compound to obtain Intermediate 1; and
subjecting Intermediate 1 to deprotection of an R₃ protecting group and then second substitution with an R₂ active compound in sequence to obtain the Glu-urea compound; wherein
one of the R₁ active compound and the R₂ active compound is selected from the group consisting of 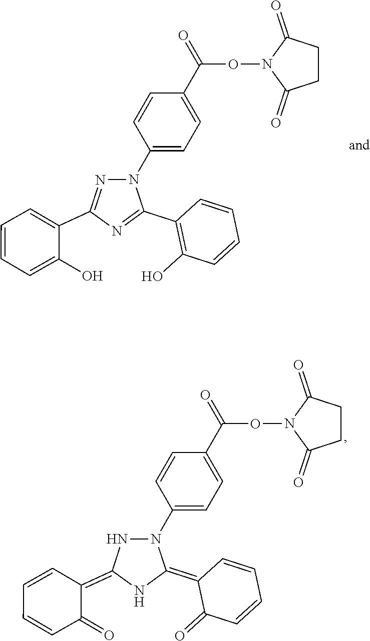 [SMILES: O=C(ON1C(=O)CCC1=O)c1ccc(-n2nc(-c3ccccc3O)nc2-c2ccccc2O)cc1 ; O=C1C=CC=C/C1=C1/N/C(=C2/C=CC=CC2=O)N(c2ccc(C(=O)ON3C(=O)CCC3=O)cc2)N1]
while the other is one selected from the group consisting of: 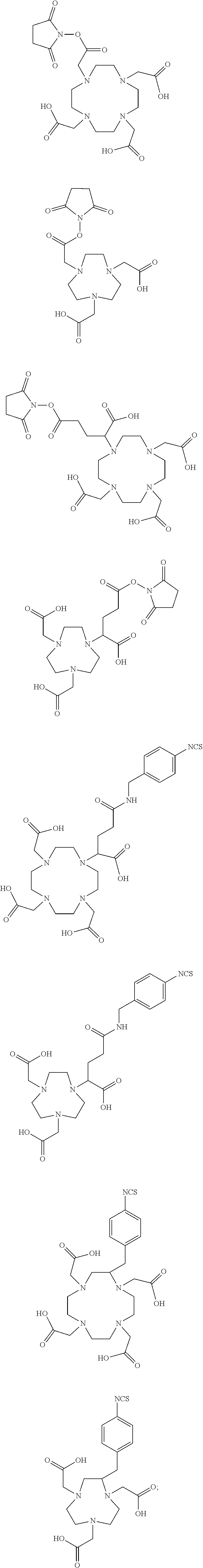
when Polypeptide compound 1 has the following structure: 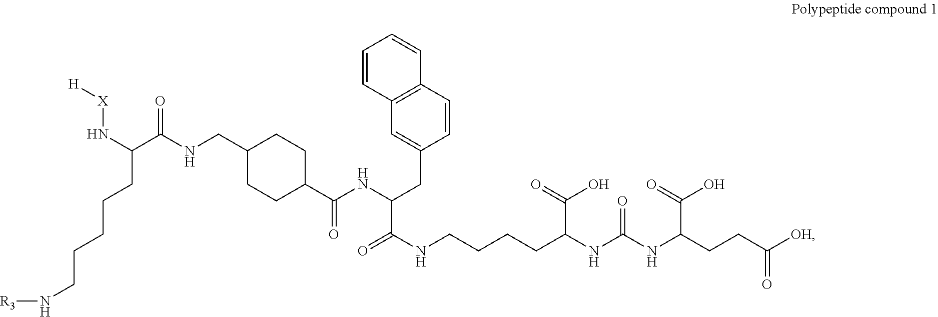
then Intermediate 1 has the following structure: 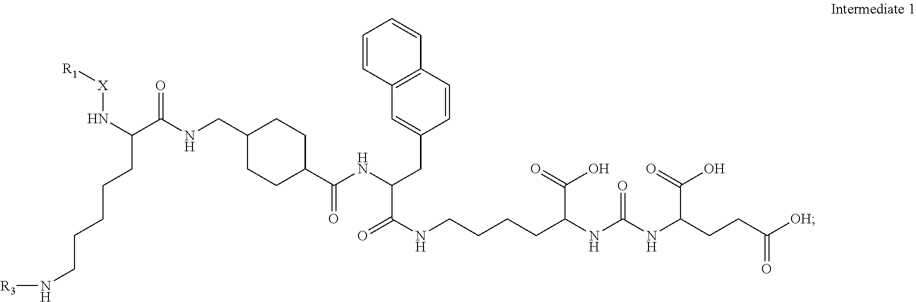
and
when Polypeptide compound 1 has the following structure: 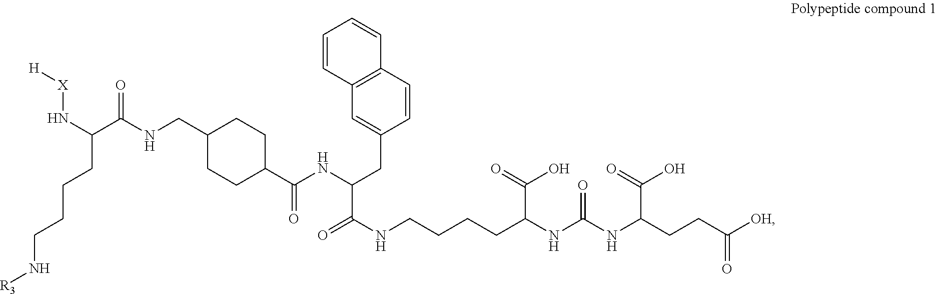
then Intermediate 1 has the following structure: 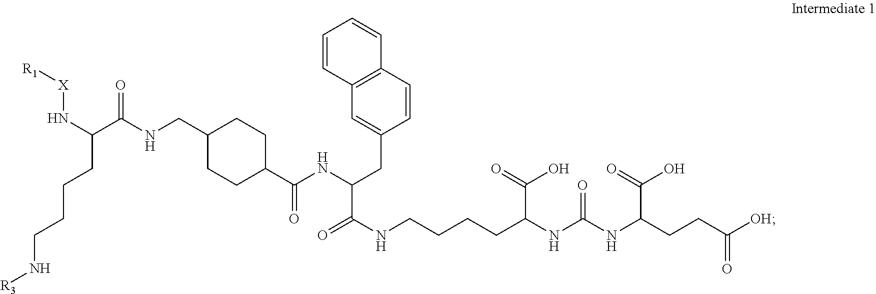
X and R₁ in Polypeptide compound 1 and Intermediate 1 are the same as X and R₁ in formula I-1 or formula I-2; and R₃ is selected from the group consisting of a Boc protecting group, a DDE protecting group, and an Fmoc protecting group; and
(ii) when X is linking group 2, the preparation method comprises the following steps:
subjecting Polypeptide compound 2 to third substitution with R₁-L to obtain Intermediate 2; and
subjecting Intermediate 2 to fourth substitution with an R₂ active compound to obtain the Glu-urea compound; wherein
L in R₁-L is one selected from the group consisting of: 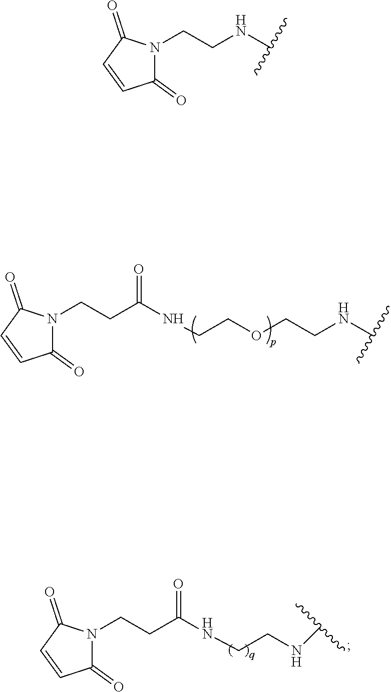
p and q in L are the same as p and q in linking group 2;
when R₁ in R₁-L is selected from the group consisting of 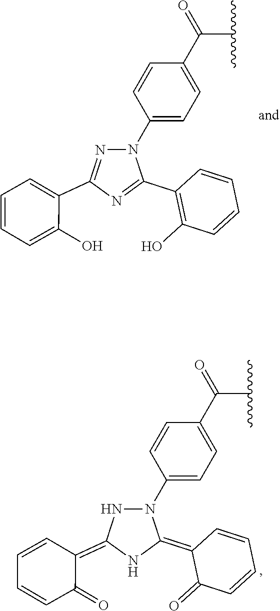 [SMILES: *C(=O)c1ccc(-n2nc(-c3ccccc3O)nc2-c2ccccc2O)cc1 ; *C(=O)c1ccc(N2N/C(=C3\C=CC=CC3=O)N/C2=C2/C=CC=CC2=O)cc1]
the R₂ active compound is one selected from the group consisting of: 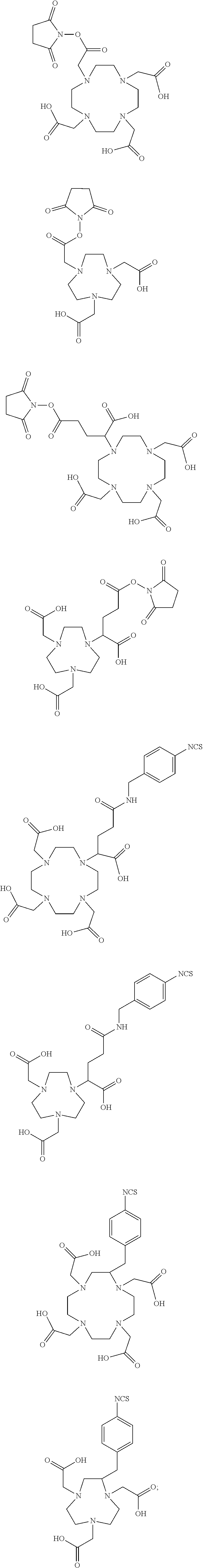
when R₁ in R₁-L is the group to be labeled, the R₂ active compound is selected from the group consisting of 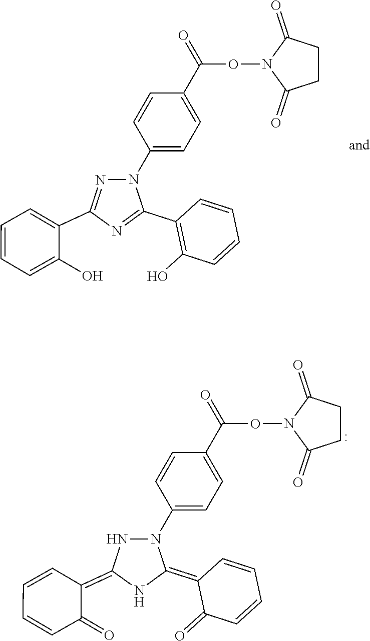 [SMILES: O=C(ON1C(=O)CCC1=O)c1ccc(-n2nc(-c3ccccc3O)nc2-c2ccccc2O)cc1 ; O=C1C=CC=C/C1=C1/N/C(=C2/C=CC=CC2=O)N(c2ccc(C(=O)ON3C(=O)CCC3=O)cc2)N1]
when Polypeptide compound 2 has the following structure: 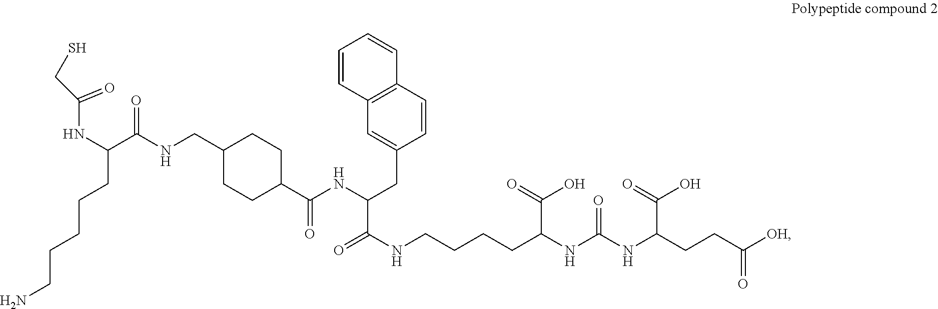 [SMILES: NCCCCCC(NC(=O)CS)C(=O)NCC1CCC(C(=O)NC(Cc2ccc3ccccc3c2)C(=O)NCCCCC(NC(=O)NC(CCC(=O)O)C(=O)O)C(=O)O)CC1]
then Intermediate 2 has the following structure: 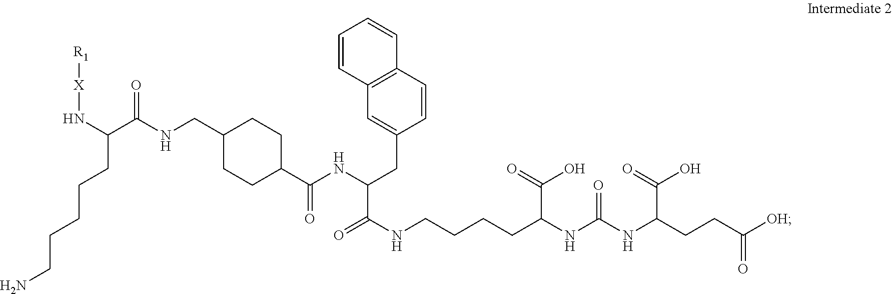
and
when Polypeptide compound 2 has the following structure: 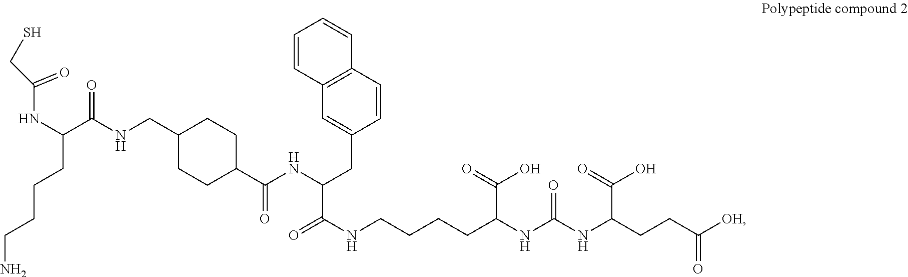 [SMILES: NCCCCC(NC(=O)CS)C(=O)NCC1CCC(C(=O)NC(Cc2ccc3ccccc3c2)C(=O)NCCCCC(NC(=O)NC(CCC(=O)O)C(=O)O)C(=O)O)CC1]
then Intermediate 2 has the following structure: 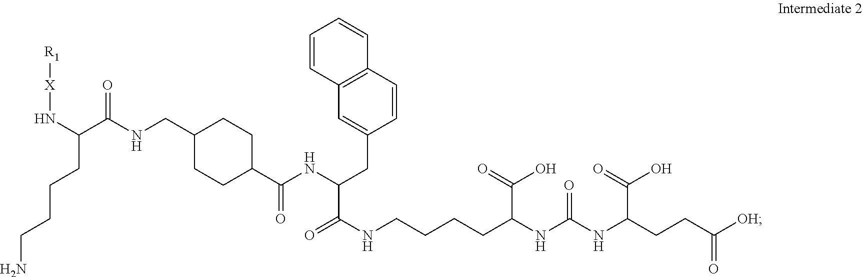
X and R₁ in Polypeptide compound 2 and Intermediate 2 are the same as X and R₁ in formula I-1 or formula I-2.

3. A pharmaceutically acceptable salt of Glu-urea compound, wherein the pharmaceutically acceptable salt is prepared by reacting a Glu-urea compound to a reaction with an acid or a base; and the Glu-urea compound is the Glu-urea compound according to claim 1.

4. A nuclide-targeted probe, wherein the nuclide-targeted probe is prepared by subjecting the chelation group in the Glu-urea compound according to claim 1 or a pharmaceutically acceptable salt of the Glu-urea compound to complexation with a nuclide.

5. The nuclide-targeted probe according to claim 4, wherein the nuclide is at least one selected from the group consisting of ¹⁸F, ⁴⁷Sc, ⁶⁴Cu, ⁶⁷Cu, ⁶⁷Ga, ⁶⁸Ga, ⁸⁹Zr, ⁸⁶Y, ⁸⁹Sr, ⁹⁰Y, ^{99m}Tc, ¹⁰⁵Rh, ¹⁰⁹Pd, ¹¹¹In, ¹¹⁹Sb, ¹⁴⁹Tb, ¹⁵³Sm, ¹⁵⁷Gd, ¹⁶¹Tb, ¹⁶⁶Ho, ¹⁷⁷Lu, ¹⁸⁶Re, ¹⁸⁸Re, ²⁰¹Tl, ²⁰³Pb, ²¹²Pb, ²¹²Bi, ²¹³Bi, ²²³Ra, ²²⁷Th, and ²²⁵Ac.

6. A preparation method of the nuclide-targeted probe according to claim 4, comprising the following steps:
subjecting the Glu-urea compound or a pharmaceutically acceptable salt thereof to complexation with the labeling nuclide to obtain the nuclide-targeted probe or a pharmaceutically acceptable salt of the nuclide-targeted probe; wherein the Glu-urea compound is the Glu-urea compound according to claim 1 or or the pharmaceutically acceptable salt of the Glu-urea compound.

7. A pharmaceutical composition, comprising an active component and a pharmaceutically acceptable auxiliary material; wherein the active component is the Glu-urea compound according to claim 1 or is is the pharmaceutically acceptable salt of the Glu-urea compound.

8. A pharmaceutical composition, comprising an active component and a pharmaceutically acceptable auxiliary material; wherein the active component is the nuclide-targeted probe according to claim 4 or is the pharmaceutically acceptable salt of the nuclide-targeted probe.

9. The nuclide-targeted probe according to claim 8, wherein the nuclide is at least one selected from the group consisting of ¹⁸F, ⁴⁷Sc, ⁶⁴Cu, ⁶⁷Cu, ⁶⁷Ga, ⁶⁸Ga, ⁸⁹Zr, ⁸⁶Y, ⁸⁹Sr, ⁹⁰Y, ^{99m}Tc, ¹⁰⁵Rh, ¹⁰⁹Pd, ¹¹¹In, ¹¹⁹Sb, ¹⁴⁹Tb, ¹⁵³Sm, ¹⁵⁷Gd, ¹⁶¹Tb, ¹⁶⁶Ho, ¹⁷⁷Lu, ¹⁸⁶Re, ¹⁸⁸Re, ²⁰¹Tl, ²⁰³Pb, ²¹²Pb, ²¹²Bi, ²¹³Bi, ²²³Ra, ²²⁷Th, and ²²⁵Ac.

10. A method for treating or diagnosing a disease mediated by prostate-specific membrane antigen (PSMA), comprising a step of administering a medicament containing the Glu-urea compound according to claim 1 to a subject in need thereof.

11. The method according to claim 10, wherein the PSMA protein-mediated disease is a tumor.
